(19) United States
(12) Patent Application Publication
(10) Pub. No.: US 2018/0207029 A1
(43) Pub. Date: Jul. 26, 2018

(54) GLAUCOMA TREATMENT METHODS AND APPARATUS

(71) Applicant: ALeyeGN Technologies LLC, Saratoga, CA (US)

(72) Inventors: Rajeev HEREKAR, Palo Alto, CA (US); Satish HEREKAR, Palo Alto, CA (US); John Randall SAMPLES, Olympia, WA (US)

(21) Appl. No.: 15/934,112

(22) Filed: Mar. 23, 2018

Related U.S. Application Data
(63) Continuation of application No. PCT/US17/50799, filed on Sep. 8, 2017.
(60) Provisional application No. 62/385,234, filed on Sep. 8, 2016.
(60) Provisional application No. 62/473,269, filed on Mar. 17, 2017.

(51) Int. Cl. A61F 9/00; A61N 5/06; A61N 7/02

(52) U.S. Cl. CPC A61F 9/00; A61N 2005/067; A61N 7/02; A61N 5/0625

(57) ABSTRACT

The methods and apparatus disclosed herein can be used to treat glaucoma of the eye. The methods and apparatus can be configured to apply energy to the sclera, cornea, and/or other regions of the eye in order to shrink collagenous tissue near Schlemm's canal. Juxtacanalicular treatment of the sclera and/or cornea adjacent Schlemm's canal can be used to dilate Schlemm's canal, collector channels, and/or the Trabecular Meshwork. The methods and apparatus can be configured to apply energy to the sclera to generate scleral vacuoles in the sclera to improve uveoscleral outflow.

### CROSS-REFERENCE

The present application is a continuation of PCT application PCT/US2017/050799, filed Sep. 8, 2017, entitled “GLAUCOMA TREATMENT METHODS AND APPARATUS” (attorney docket no. 48848-705.602), which claims priority to U.S. App. Ser. No. 62/385,234, filed Sep. 8, 2016, entitled “EFFECTIVE OCULAR LENS POSITIONING AND GLAUCOMA TREATMENT METHODS AND APPARATUS” (attorney docket no. 48848-705.102), and U.S. App. Ser. No. 62/473,269, filed Mar. 17, 2017, entitled “GLAUCOMA TREATMENT METHODS AND APPARATUS” (attorney docket no. 48848-705.103), the entire disclosures of which are incorporated herein by reference.

The subject matter of the present application is related to PCT application PCT/US2017/023092, filed on 17 Mar. 2017, entitled “EFFECTIVE OCULAR LENS POSITIONING METHODS AND APPARATUS” (attorney docket no. 48848-705.601), the entire disclosure of which is incorporated herein by reference.

The subject matter of the present application is related to PCT application PCT/US2016/055829, filed on 6 Oct. 2016, entitled “ULTRASOUND DIRECTED CAVITATIONAL METHODS AND SYSTEMS FOR OCULAR TREATMENTS” (attorney docket no. 48848-704.601), the entire disclosure of which is incorporated herein by reference.

The subject matter of the present application is related to PCT/US2014/023763, filed 11 Mar. 2014, entitled “Scleral translocation elasto-modulation methods and apparatus” (attorney docket no. 48848-703.601), the entire disclosure of which is incorporated herein by reference.

### BACKGROUND OF THE INVENTION

Glaucoma treatment of the eye has proven challenging. Glaucoma is disease related to intraocular pressure (IOP) of the eye. Glaucoma can lead to chronic, progressive deterioration of the optic nerve caused or worsened by intraocular pressure. Prior methods and apparatus can be less than ideal in at least some respects. Many of the prior approaches can be more complex and/or invasive than would be ideal, and may require incisions of the eye and surgery to treat glaucoma. For example, therapies such as trabeculectomy surgery or implantation of glaucoma drainage devices can require invasive surgical intervention and potentially have adverse safety risks in some instances. Treatment to reduce IOP with medicated eye drops can be less than ideal due to lack of patient compliance. Also, some IOP medications can be related to side effects in some instances. In light of the above, improved methods and apparatus of treating glaucoma are needed. Ideally, such methods and apparatus would be less invasive than some of the prior treatments and provide successful reduction in IOP.

### SUMMARY OF THE INVENTION

The methods and apparatus disclosed herein can be used to treat glaucoma of the eye, and can be combined with other treatments from a treatment system. The glaucoma treatment can be combined with refractive treatment of the eye, presbyopia treatment of the eye, and combinations thereof or provided with a separate treatment. The sclera can be treated in order to shrink or relax the sclera, and combinations thereof in order to treat glaucoma. The energy can be applied to the eye in many ways, for example with one or more of laser energy, ultrasound energy or radiofrequency (RF) energy. The energy can be delivered with an optical or ultrasound delivery system. The system may comprise an energy delivery system to direct energy to the eye or a hand held probe to direct energy to the eye. Although reference is made to generally annular energy treatment patterns, the energy treatment pattern can be delivered in many ways, and may comprise a portion of an annulus, a polygon, or interspersed treatment along a generally circumferential pattern along the treatment region of the eye. The glaucoma treatment may comprise a first generally annular treatment region to dilate Schlemm's canal, and a second approximately annular treatment pattern to improve outflow through the sclera with increased porosity of the sclera. The improved porosity of the sclera can be provided with vacuoles formed in the sclera or with vacuoles having an increased size or density in the sclera.

In a first aspect, a system for treating glaucoma of an eye comprises a processor, an energy source configured to generate energy to treat the eye, and a scanner operably coupled to the energy source and the processor, the scanner configured to deliver the energy to the plurality of treatment locations in order to stretch the Schlemm's canal of the eye. The processor is configured with instructions to: receive input corresponding to a plurality of locations of a Schlemm's canal of the eye, and generate a plurality of treatment locations for the eye in response to the plurality of locations, wherein the plurality of treatment locations is located within 2 mm of the Schlemm's canal, and located radially inward from the Schlemm's canal toward an optical axis of the eye and radially outward from the Schlemm's away from the optical axis of the eye.

The plurality of treatment locations may comprise juxtacanalicular locations located within 2 mm of the Schlemm's canal. The plurality of treatment locations may extend in a first annular treatment pattern on a first side of the Schlemm's canal and a second annular treatment pattern on a second side of Schlemm's canal opposite the first side in order stretch tissue between the first annular treatment pattern and the second annular treatment pattern to dilate the Schlemm's canal and increase porosity of a trabecular meshwork of the eye.

The first annular treatment pattern may be located radially inward from Schlemm's canal and the second annular treatment pattern may be located radially outward from Schlemm's canal relative to the optical axis of the eye.

The processor may be configured with instructions to shrink tissue with the first annular treatment pattern prior to shrinking tissue with the second annular treatment pattern.

The processor may be configured with instructions to shrink tissue with at least a portion of the first annular treatment pattern prior to shrinking tissue with at least a portion of the second annular treatment pattern.

The first annular treatment pattern may comprise a first plurality of spaced apart annular treatment patterns and the second annular treatment pattern may comprise a second plurality of spaced apart annular treatment patterns. The first plurality of spaced apart treatment annular treatment patterns may optionally comprise angularly separated spaced apart treatment patterns and the second plurality of annular treatment pattern may optionally comprise angularly separated spaced apart treatment patterns. The first plurality of spaced apart treatment annular treatment patterns may optionally comprise radially separated spaced apart treatment patterns and the second plurality of annular treatment pattern may optionally comprise radially separated spaced apart treatment patterns.

The processor may be configured with instructions to configured repeatedly deliver the energy to each the plurality of treatment locations with a time delay in order to fractionate delivery of energy to said each of the plurality of treatment locations. The time delay may be within a range from about 10 millisecond (ms) to about 60 seconds. The time delay may be within a range from about 100 ms to about 30 seconds. The time delay may be within a range from about 500 ms to about 15 seconds. The time delay may be within a range from about 1 second (s) to about 10 seconds.

The processor coupled to the energy source and the scanner may be configured with instructions to heat tissue at the plurality of treatment locations to a temperature within a range from 60 to 70 degrees Centigrade at a depth within a range from 50 to 400 microns at each of the plurality of treatment locations along the first annular pattern and the second annular pattern.

A majority of a treatment energy of the first treatment pattern may be located within 1.5 mm of the Schlemm's canal and a majority of treatment energy of the second pattern may be located within 1.5 mm of Schlemm's canal.

The first annular treatment pattern and the second annular treatment pattern may optionally be configured to open an angle of the eye by an amount within a range from 1 to 6 degrees.

The first annular treatment pattern may extend at least about 30 degrees around the optical axis of the eye and the second annular treatment pattern may extend at least about 30 degrees around the optical axis of the eye. The first treatment annular treatment pattern may extend at least about 40 degrees around the optical axis of the eye and the second treatment pattern may extend at least about 40 degrees around the optical axis of the eye.

The first annular treatment pattern and the second annular treatment pattern may be arranged avoid heating tissue overlaying the Schlemm's canal.

The first annular treatment pattern and the second annular treatment pattern may comprise circular, oval, elliptical, egg-like, non-circular, non-elliptical, or asymmetrical, shapes patterned so as to correspond to the shape of Schlemm's canal or the limbus.

The energy source may comprise one or more of a pulsed laser, a continuous laser, a pulsed ultrasound transducer, a HIFU array, or a phased HIFU array.

The input may comprise an input from a user of the system or an input from an imaging apparatus.

The processor may be configured with instructions to generate vacuoles in a sclera of the eye. The energy source may comprise a laser having a wavelength within a range from about 1.9 to 2.3 microns. The energy source may comprise a laser having a wavelength of about 1.9 microns. The processor may be configured with instructions to scan laser beam with an annular pattern on the sclera. The processor may be configured with instructions to scan the sclera with two repetitions of the annular pattern.

The processor may be configured with instructions to treat the eye in order to increase an angle of the eye, dilate and stretch one or more of the trabecular meshwork or Schlemm's canal, increasing porosity of the sclera and dilating the perilimibic sclera, dilate collector channels, or dilate ostia of collector channels of the eye.

In another aspect, a system to treat glaucoma of an eye comprises an energy source and a handpiece coupled to the energy source. The handpiece comprises an eye contacting surface to couple to the eye and a plurality of energy releasing elements disposed at a plurality of locations to release energy to the eye at a plurality of treatment locations within 2 mm of a Schlemm's canal of the eye. the plurality of locations correspond to treatment locations located radially inward from the Schlemm's canal toward an optical axis of the eye and radially outward from the Schlemm's away from the optical axis of the eye as measured along an exterior surface of the eye.

The first plurality of locations may comprise a first plurality of locations located radially inward from a second plurality of locations, the first plurality of locations arranged in a first annular treatment pattern to treat the eye radially inward from the Schlemm's canal and the second plurality of locations arranged to treat the eye with a second annular treatment pattern radially outward from the Schlemm's canal of the eye. The first annular treatment pattern may correspond to a first diameter within a range from about 10 mm to about 12 mm and the second annular treatment pattern may correspond to a second diameter within a range from about 12 mm to about 14 mm.

The plurality of energy releasing elements may comprise a plurality of optical fibers and the energy source comprises a laser. The plurality of energy releasing elements may comprise a plurality of electrodes and the energy source may comprise an electroporation energy source, a microwave energy source, a thermal energy source, an electrical energy source, an electrophoretic energy source, or a di-electrophoretic energy source.

The system may further comprise a processor coupled to the energy source to deliver energy to the plurality of treatment locations. The processor may be configured to fractionate energy delivered to each of the plurality of treatment locations.

In another aspect, a method for treating glaucoma of an eye comprises determining a plurality of locations of a Schlemm's canal of the eye and delivering energy to a plurality of treatment locations of the eye in response to the plurality of locations. The plurality of treatment locations is located within 2 mm of the Schlemm's canal, and located radially inward from the Schlemm's canal toward an optical axis of the eye and radially outward from the Schlemm's canal away from the optical axis of the eye. The energy is delivered to the plurality of treatment locations in order to stretch the Schlemm's canal of the eye.

The plurality of treatment locations may comprise juxtacanalicular locations located within 2 mm of the Schlemm's canal. The plurality of treatment locations may extend in a first annular treatment pattern on a first side of the Schlemm's canal and a second annular treatment pattern on a second side of Schlemm's canal opposite the first side. The first annular treatment pattern may be located radially inward from Schlemm's canal and the second annular treatment pattern may be located radially outward from Schlemm's canal relative to the optical axis of the eye. The tissue may shrink with the first annular treatment pattern and the second annular treatment pattern in order stretch tissue between the first annular treatment pattern and the second annular treatment pattern to dilate the Schlemm's canal and increase porosity of a trabecular meshwork of the eye.

The tissue may be heated to a temperature within a range from 60 to 70 degrees Centigrade at a depth within a range from 50 to 400 microns at each of the plurality of treatment locations along the first annular pattern and the second annular pattern.

A majority of a treatment energy of the first treatment pattern may be located within 1.5 mm of the Schlemm's canal and a majority of treatment energy of the second pattern may be located within 1.5 mm of Schlemm's canal.

The first annular treatment pattern and the second annular treatment pattern may be configured to open an angle of the eye by an amount within a range from 1 to 3 degrees.

The first annular treatment pattern may extend at least about 90 degrees around the optical axis of the eye and the second annular treatment pattern may extend at least about 90 degrees around the optical axis of the eye. The first treatment annular treatment pattern may extend at least about 180 degrees around the optical axis of the eye and the second treatment pattern may extend at least about 180 degrees around the optical axis of the eye.

The first annular treatment pattern and the annular second treatment may be arranged avoid shrinking tissue overlaying the Schlemm's canal.

The first annular treatment pattern and the second annular treatment pattern may comprise circular, oval, elliptical, egg-like, non-circular, non-elliptical, or asymmetrical, shapes patterned so as to correspond to the shape of Schlemm's canal or the limbus.

The energy may comprise energy from one or more of a pulsed laser, a continuous laser, a pulsed ultrasound transducer, a HIFU array, or a phased HIFU array.

The method may further comprise generating vacuoles in a sclera of the eye.

The energy may comprise energy from a laser having a wavelength within a range from about 1.9 to 2.3 microns.

A laser beam may be scanned with an annular pattern on the sclera.

The processor may be configured with instructions to scan the sclera with two repetitions of the annular pattern.

In another aspect, an apparatus to treat glaucoma of an eye having a Schlemm's canal comprises an energy source and a processor coupled to the energy source. The processor is configured with instructions to shrink collagenous tissue near the Schlemm's canal.

The energy source may comprise a laser. The energy source may comprise a laser having wavelength within a range from about 1.5 um to about 2.1 um.

The energy source comprise an amount of energy per unit time (power) to the eye within a range from about 50 mW to about 900 mW, preferably within a range from about 100 to about 700 mw, more preferably within a range from about 200 to 400 mW.

The energy source may comprise one or more of a pulsed laser, a continuous laser, a pulsed ultrasound transducer, a HIFU array, or a phased HIFU array

The processor may be configured with instructions to apply a total amount of energy applied to the eye to treat glaucoma within a range from about 4 J to about 90 J, preferably within a range from about 5 J to about 50 J, with a treatment time within a range from about 4 to 200 seconds, preferably within a range from about 8 to 100 seconds, and optionally wherein the energy source comprises an ultrasound energy source or a laser.

The processor may be configured with instructions to scan the energy source along the eye with a scan rate within a range from about 10 to 100 mm/second, preferably within a range from about 12 to 50 mm per second, more preferably within a range from about 20-30 mm/second, for example, about 25 mm/second. The energy source may optionally comprise an ultrasound energy source or a laser.

The energy source may comprise a laser, and wherein the laser comprises a cross-sectional beam spot size within a range from about 100 to 500 um, preferably within a range from about 150-400 um, more preferably within a range from about 200-300 um spot size when applied to the tissue near Schlemm's canal.

The energy source may comprise a laser or an ultrasound energy source. The treatment may be within a range from about 8 to about 100 seconds.

The energy source may comprise an ultrasound transducer.

The processor may be configured with instructions to direct energy into the eye with an annular pattern, the annular pattern comprising an inner dimension located radially inward from Schlemm's canal and an outer dimension radially outward from Schlemm's canal.

The processor may be configured with instructions to treat scleral tissue of the eye to provide vacuoles in the sclera and increase outflow through the sclera in order to lower intraocular pressure.

The processor may be configured with instructions to treat the eye in order to increase an angle of the eye, dilating and stretching one or more of the trabecular meshwork or Schlemm's canal, increasing porosity of the sclera and dilating the perilimbic sclera.

The apparatus may further comprise a lens having a concavely curved surface to contact the eye and conduct heat from tissue heated with the energy source.

In another aspect, an apparatus to treat an eye comprises a laser to generate a beam, a scanner to direct the beam, and a processor coupled to the laser and the beam, the processor comprising instructions treat glaucoma of the eye with a first pattern and a refractive error of the eye with a second pattern.

In another aspect, a method may comprise providing any of the treatment apparatus described herein.

In another aspect, a system for treating an eye comprising a Schlemm's canal comprises a processor, configured with instructions to: receive an anterior image of the eye generated with a camera anterior to the eye; estimate a plurality of Schlemm's canal locations in response to the anterior image of the eye or a plurality of optical coherence tomography (OCT) images of the eye; determine a plurality of treatment locations for the eye in response to the plurality of the Schlemm's locations canal; and overlay of the plurality of treatment locations and the plurality of Schlemm's canal locations on the anterior image of the eye shown on a display.

In another aspect, a system for treating an eye comprises a processor, an energy source configured to generate energy for treating the eye, and a scanner operably coupled to the energy source and the processor, the scanner configured to deliver the energy to the plurality of treatment locations. The processor is configured with instructions to: estimate a plurality of locations of a Schlemm's canal of the eye; and generate a plurality of treatment locations for the eye in response to the plurality of locations, wherein the plurality of treatment location is located within 2 mm of the Schlemm's canal, and located radially inward from the Schlemm's canal toward an optical axis of the eye or radially outward from the Schlemm's away from the optical axis of the eye as measured along an exterior surface of the eye.

In another aspect, a method for treating an eye, the eye comprising a Schlemm's canal, comprises receiving, with a processor, an anterior image of the eye generated with a camera anterior to the eye; estimating, with the processor, a plurality of Schlemm's canal locations in response to the anterior image of the eye or a plurality of optical coherence tomography (OCT) images of the eye; determining, with the processor, a plurality of treatment locations for the eye in response to the plurality of Schlemm's canal locations; and overlaying, with the processor, the plurality of treatment locations and the plurality of Schlemm's canal locations on the anterior image of the eye.

In any of the aspects described herein, the processor may be further configured to register the plurality of locations of Schlemm's canal with a corresponding plurality of anterior image locations.

In any of the aspects described herein, the plurality of treatment locations is located on either side of the plurality of Schlemm's canal locations.

The plurality of treatment locations may comprise a first plurality of treatment locations positioned radially inwardly from the plurality of Schlemm's canal locations toward an optical axis of the eye and a second plurality of treatment locations located radially outwardly from the plurality of Schlemm's canal locations.

Each of the first plurality of treatment locations may be within a range from about 0.5 mm to about 4 mm of the second plurality of treatment locations. Energy pulses at the first plurality of treatment locations optionally may not overlap with the second plurality of treatment locations. The first plurality of pulses may optionally overlap with each other to define a first arc and the second plurality of pulses may optionally overlap with each other to define a second arc with a gap between the first arc and the second arc.

The first plurality of treatment locations may comprise overlapping treatment locations axis. The second plurality of treatment locations may comprise overlapping treatment locations.

The processor may be configured with instructions to alternate treatment at the first plurality of treatment locations with treatment at the second plurality of treatment locations.

Alternating the treatment between the first plurality of treatment locations and the second plurality treatment locations may decrease movement of tissue at a location between the first plurality of treatment locations and the second plurality of treatment locations.

Alternating the treatment between the first plurality of treatment locations and the second plurality treatment may inhibit biasing of tissue at the first plurality of treatment locations or the second plurality of treatment locations.

Energy from an energy source may be delivered in a treatment sequence alternating between the first plurality of treatment locations and the second plurality of treatment locations along a scan path defined by successive distances between each of the plurality of treatment locations and an immediately prior treatment location of the sequence. The scan path may comprise a distance greater than a combined distance defined by a first combined distance of the first plurality of treatment locations and second combined distance of the second plurality of treatment locations. The scan path may comprise at least about 1.1 times the combined distance. The scan path may comprise at least about 1.25 times the combined distance. The first plurality of treatment locations may define a first perimeter. The second plurality of treatment locations may define a second perimeter. The first perimeter may comprise the first combined distance and the second perimeter comprises the second combined distance.

Alternating the treatment between the first plurality of treatment locations and the second plurality treatment locations may comprise treating a first portion of the first plurality of treatment locations followed by a first portion of the second plurality of treatment locations followed by a second portion of the first plurality of treatment locations. Locations of the second portion of the first plurality of treatment locations may be interspersed between locations of the first portion of the first plurality of treatment locations. Locations of the second portion of the first plurality of treatment locations may not be interspersed between locations of the first portion of the first plurality of treatment locations, the first portion defining a first perimeter, the second portion defining a second perimeter. The first portion may optionally define a first annular pattern and a second annular pattern radially separated from each other with reference to an optical axis of the eye.

Tissue at the first plurality of treatment locations and the second plurality of treatment locations may shrink in response to energy from an energy source. Tissue between the first plurality of treatment locations and the second plurality of treatment locations may be stretched in order to dilate tissue selected from the group consisting of the Schlemm's canal, a trabecular meshwork and collector channels of the eye. Tissue at the first plurality of treatment locations may be heated to a temperature within a range from 60 to 70 degrees Centigrade at a depth within a range from 50 to 400 microns. An epithelium through which energy is transmitted may remain substantially intact. Tissue at the first plurality of treatment locations may comprise corneal tissue heated to a temperature within a range from 60 to 70 degrees Centigrade at a depth within a range from 50 to 300 microns. Tissue at the second plurality of treatment locations may comprise scleral tissue heated to a temperature within a range from 60 to 70 degrees Centigrade at a depth within a range from 50 to 400 microns. An endothelial layer of the cornea beneath the first plurality of treatment zones may remain substantially intact and the cornea at the plurality of treatment locations may remain substantially clear subsequent to treatment with the energy source. The cornea may optionally remain substantially clear at the plurality of treatment locations for over a period of time extending from one hour to one day subsequent to treatment with the energy source.

In any of the aspects described herein, each of the plurality of treatment locations may correspond to a location on the anterior image. The processor may be configured with instructions to generate a treatment table, the treatment table comprising a plurality of coordinate reference locations corresponding to the plurality of treatment locations overlaid on the anterior image. The energy source may optionally comprise a pulsed energy source and wherein each of the plurality of coordinate references corresponds to a pulse from an energy source.

In any of the aspects described herein, the processor may be configured to generate instructions to treat the plurality of treatment locations with a treatment energy. The treatment energy may be absorbed by an epithelial layer of the eye. Over 90% of the treatment energy transmitted through the epithelial layer may be absorbed within about 2 mm of the epithelial layer. The energy source and the treatment table may be configured to shrink tissue at each of the plurality of treatment locations on either side of the Schlemm's canal to dilate the Schlemm's canal and stretch tissue adjacent the Schlemm's canal in response to tissue shrinkage on either side of Schlemm's canal. The shrunken tissue may be selected from the group consisting of a trabecular meshwork of the eye, the Schlemm's canal of the eye or collector channels of the eye.

In any of the aspects described herein, the plurality of Schlemm's Canal locations may be estimated in response to a plurality of limbus locations of the eye. The plurality of limbus locations may be determined in response to changes in intensity of the anterior image.

In any of the aspects described herein, Schlemm's Canal may comprise a non-circular, non-elliptical shape.

In any of the aspects described herein, the plurality of treatment locations may be shown on the display in relation to the plurality of Schlemm's canal locations. The plurality of treatment locations may comprise a first plurality of treatment locations located radially inward from the Schlemm's canal toward an optical axis of the eye and a second plurality of treatment locations located radially outward from the Schlemm's canal away from the optical axis of the eye.

Any of the aspects described herein may further comprise a display configured to show the anterior image of the eye.

Any of the aspects described herein may further comprise a camera configured to generate the anterior image of the eye. The camera may comprise a video camera configured to capture images of the eye. The processor may be configured to overlay the plurality of treatment locations on the images shown on the display in real-time.

Any of the aspects described herein may further comprise an energy source configured to generate energy for treating the eye. The energy source may be selected from the group consisting of a laser energy source, an ultrasound energy source, an electroporation energy source, a microwave energy source, a thermal energy source, an electrical energy source, an electrophoretic energy source, and a di-electrophoretic energy source. The energy source may be configured to deliver energy to the treatment location in accordance with a user's instructions. The processor may be configured to generate a third plurality of treatment locations located radially outward from the second plurality of treatment locations to generate vacuoles or increase a size of vacuoles in a sclera of the eye. The energy source may be configured to generate vacuoles and/or increase a size of vacuoles in a sclera of the eye radially outward from the second plurality of treatment locations in order to increase uveal scleral outflow of the eye. The energy source may comprise a laser. The laser may comprise a continuous laser. The laser may comprise a pulsed laser. The laser may be configured to generate a first light beam having a wavelength within a first range from about 1.4 to 1.6 um and second light beam having a second wavelength within a range from about 1.9 to 2.3 um The laser may be configured to generate a first light beam having a wavelength of about 1.47 um and second light beam having a second wavelength of about 2.01 um. The energy source may comprise a first laser to generate the first light beam and a second laser to generate the second light beam. A scanner may optionally be operably coupled to the energy source and configured to deliver the energy to the plurality of treatment locations.

In any of the aspects described herein, each of the plurality of OCT images may comprise a slice along a plane of tissue of the eye. Each of the plurality of slices may be registered with the eye to determine the plurality of locations of the Schlemm's canal along a two-dimensional path. Each of the slices may be rotated about an optical axis of the eye with respect to other slices in order to determine the two dimensional path of Schlemm's canal around the optical axis of the eye. The plurality of treatment locations may comprise a treatment pattern shown on a two-dimensional anterior image of the eye. A two-dimensional (“2D”) treatment pattern projected onto the eye may comprise a plurality of locations on either side of Schlemm's canal on anterior layer of the eye. The anterior layer may be selected from the group consisting of a cornea of the eye and a sclera of the eye.

In any of the aspects described herein, the system may be configured to treat the eye for a period of time no more than a minute in a single treatment session.

In any of the aspects described herein, the camera may comprise a video camera optically coupled to an operating microscope.

Any of the aspects described herein may further comprise an optical coherence tomography (OCT) system configured to capture the OCT image of the eye. The OCT system may comprise a real-time OCT imaging system capable of generating OCT images at a rate of one frame per second. The OCT system may be coupled to the laser system with a shared support. The OCT system may comprise an OCT system physically separated from the treatment system. The processor may be configured to receive data from the OCT system.

In another aspect, a system for treating glaucoma of an eye comprises a processor, an energy source configured to generate energy to treat the eye, and a scanner operably coupled to the energy source and the processor, the scanner configured to deliver the energy to the plurality of treatment locations in order to stretch the Schlemm's canal of the eye. The processor is configured with instructions to generate a plurality of treatment locations for the eye, wherein the plurality of treatment location is located within 2 mm of the Schlemm's canal, and located radially inward from the Schlemm's canal toward an optical axis of the eye and radially outward from the Schlemm's away from the optical axis of the eye.

In another aspect, a system for treating glaucoma of an eye comprises an energy source and a handpiece coupled to the energy source. The handpiece comprises an eye contacting surface to couple to the eye and a plurality of energy releasing elements disposed at a plurality of locations to release energy to the eye at a plurality of treatment locations. The plurality of locations correspond to treatment locations located radially outward from the Schlemm's away from the optical axis of the eye as measured along an exterior surface of the eye.

Any of the aspects described herein may further comprise a user interface comprising one or more fields to receive input data from a user which is used to configure and adjust the plurality of treatment locations generated by the processor.

In any of the aspects described herein the processor may be configured with instructions to fractionate delivery of the energy to each of the plurality of treatment locations with repeated delivery of energy to said each of the plurality of treatment locations. The fractionation optionally may comprise an amount of fractionation within a range from about 0.1% to about 10%, optionally wherein the range is from about 0.2% to about 5%. The fractionation may optionally correspond to an amount of exposure time for said each location and a time delay between successive exposures to said plurality of locations.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 illustrates an eye, in accordance with embodiments;

FIG. 2 illustrates stabilization of an eye by cross-linking to treat presbyopia, in accordance with embodiments;

FIG. 3 illustrates common fluid outflow paths of the eye including the location of the limbus and Schlemm's Canal, in accordance with embodiments;

FIG. 4A illustrates a treatment pattern which may be used to dilate Schlemm's canal, in accordance with embodiments;

FIG. 4B shows a cross-section of the eye of FIG. 4A taken along line B-B with treatment annuli located on either side of Schlemm's canal, in accordance with embodiments;

FIG. 4C shows a magnified view of the dashed box indicated in FIG. 4B.

FIGS. 5A-5B show possible juxtacanalicular treatment patterning which may be used to treat glaucoma and dilate Schlemm's canal, in accordance with embodiments;

FIGS. 6A-6B show the results of a finite element analysis simulation of juxtacanalicular shrinkage treatment of Schlemm's canal, in accordance with embodiments;

FIG. 7 illustrates an STEM adjustment system for treating an eye, in accordance with embodiments;

FIG. 8 shows a schematic of a treatment system, in accordance with embodiments;

FIGS. 9A-9C show an embodiment of a handheld probe, in accordance with embodiments;

FIG. 10 shows an image of an eye taken a camera after docking the patient interface and system to the eye, in accordance with embodiments;

FIG. 11 shows an imaging scheme which may be used to estimate the shape of Schlemm's canal, in accordance with embodiments;

FIGS. 12A-12D show an exemplary process for generating a treatment pattern based on one or more locations of the limbus, in accordance with embodiments;

FIG. 13 shows a schematic of a display for use in directing treatment to targeted treatment zones, in accordance with embodiments;

FIG. 14 shows a graphical user interface of the treatment system, in accordance with embodiments;

FIG. 15 shows a graphical user interface of the treatment system, in accordance with embodiments;

FIGS. 16-21 show a graphical user interface of the treatment system in accordance with embodiments;

FIG. 22 shows a flowchart of a method for determining a target treatment location, in accordance with embodiments;

FIG. 23 illustrates a heat sink placed over the eye of FIG. 2, in accordance with embodiments;

FIGS. 24A-24C show a structure for coupling an energy source to a surface of an eye, in accordance with embodiments;

FIG. 25 shows temperature profiles of an eye treated with a laser beam with the eye coupled to a chilled lens, in accordance with embodiments;

FIG. 26 shows a treatment system for STEM adjustment, in accordance with embodiments;

FIG. 27 shows a STEM adjustment system, in accordance with embodiments;

FIG. 28 shows a HIFU array coupled to an imaging apparatus, in accordance with embodiments;

FIG. 29 shows another HIFU array coupled to an imaging apparatus, in accordance with embodiments;

FIG. 30 shows a schematic of a one-dimensional HIFU system, in accordance with embodiments;

FIG. 31 shows femto-laser induced Schlemm's Canal-mimicking slits generated in porcine corneas, in accordance with embodiments;

FIGS. 32A-32B show schematics of test patterns used to treat porcine corneas to induce axial movement of femto-slits, in accordance with embodiments;

FIG. 33 shows STEM-induced translocation of femto-slits in porcine eyes used to represent Schlemm's Canal in order to show that glaucoma treatment open Schlemm's Canal, in accordance with embodiments;

FIGS. 34A and 34B show the pre-operative and post-operative locations respectively, of femto-slits in a porcine eye with STEM-induced translocation via 360 degree annular treatment at multiple locations, in accordance with embodiments;

FIGS. 35A and 35B show the pre-operative and post-operative locations respectively, of femto-slits in a porcine eye with STEM-induced translocation via 360 degree annular treatment at multiple locations, in accordance with embodiments;

FIGS. 36A and 36B show the pre-operative and post-operative locations respectively, of femto-slits in a porcine eye with STEM-induced translocation via 360 degree annular treatment at multiple locations, in accordance with embodiments;

FIGS. 37A and 37B show the pre-operative and post-operative locations respectively, of femto-slits in a porcine eye with STEM-induced translocation via 360 degree annular treatment at multiple locations, in accordance with embodiments;

FIGS. 38A and 38B show the pre-operative and post-operative locations respectively, of femto-slits in a porcine eye with STEM-induced translocation via 360 degree annular treatment at multiple locations, in accordance with embodiments;

FIGS. 39A and 39B show the pre-operative and post-operative locations respectively, of femto-slits in a porcine eye with STEM-induced translocation via 360 degree annular treatment at multiple locations, in accordance with embodiments;

FIGS. 40A and 40B show a zoomed-in view of the pre-operative and post-operative locations respectively, of femto-slits in a porcine eye with STEM-induced translocation via 360 degree annular treatment at multiple locations, in accordance with embodiments;

FIG. 41A shows a porcine eye prior to sub-conjunctiva 360 degree STEM shrinkage treatment about the limbus above the roof of Schlemm's Canal to open Schlemm's Canal and the Trabecular Meshwork and within the sclera to increase porosity and dilate vacuoles of the perilimbic sclera, in accordance with embodiments;

FIGS. 41B-41E show the eye of FIG. 41A after STEM treatment to open the angle, dilate supraciliary vacuoles of the sclera, and open Schlemm's Canal, in accordance with embodiments;

FIG. 42 shows an OCT image of a porcine eye treated using the glaucoma treatment system described herein, in accordance with embodiments;

FIGS. 43A-43B show the diameter of the limbus of a porcine eye, as a reference for Schlemm's canal, before and after paralimbal treatment of Schlemm's canal, in accordance with embodiments;

FIGS. 44A-44B show results of a first eye treated to widen Schlemm's canal, in accordance with embodiments;

FIGS. 45A-45B show results of a second eye treated to widen Schlemm's canal, in accordance with embodiments;

FIGS. 46A-46B show results of a third eye treated to widen Schlemm's canal, in accordance with embodiments;

FIGS. 47A-47B show OCT images of porcine eyes treated at multiple locations with the laser system, in accordance with embodiments;

FIGS. 48A-48B show pre-operative images of a porcine eye, in accordance with embodiments;

FIGS. 48C-48D show post-operative images of the eye of FIGS. 48A-48B treated juxtacanalicularly, in accordance with embodiments;

FIGS. 49A-49B show pre-operative images of a porcine eye, in accordance with embodiments;

FIGS. 49C-49D show post-operative images of the eye of FIGS. 49A-49B treated juxtacanalicularly, in accordance with embodiments;

FIGS. 50A-50B show pre-operative images of a porcine eye, in accordance with embodiments; and

FIGS. 50C-50D show post-operative images of the eye of FIGS. 50A-50B treated juxtacanalicularly, in accordance with embodiments.

### DETAILED DESCRIPTION OF THE INVENTION

A better understanding of the features and advantages of the present disclosure will be obtained by reference to the following detailed description that sets forth illustrative embodiments, in which the principles of embodiments of the present disclosure are utilized, and the accompanying drawings.

Although the detailed description contains many specifics, these should not be construed as limiting the scope of the disclosure but merely as illustrating different examples and aspects of the present disclosure. It should be appreciated that the scope of the disclosure includes other embodiments not discussed in detail above. Various other modifications, changes and variations which will be apparent to those skilled in the art may be made in the arrangement, operation and details of the method and apparatus of the present disclosure provided herein without departing from the spirit and scope of the invention as described herein.

The embodiments disclosed herein can be combined in one or more of many ways to provide improved methods and apparatus for treating the eye. The treated ocular tissue, or membranes or pathological transformations thereof, may comprise one or more of corneal tissue, lens tissue, scleral tissue, vitreal tissue, or zonulae extending between the lens capsule and the ora serrata.

As used herein like characters identify like elements.

As used herein A and/or B encompasses one or more of A or B, and combinations thereof such as A and B.

The embodiments as disclosed herein provide improved methods and apparatus for the treatment of glaucoma. The treatments and apparatus disclosed herein can be combined with many known methods and apparatus for treatment. For example, the methods and apparatus as disclosed herein can be combined with one or more known glaucoma therapies. Alternatively or in combination, the methods and apparatus as disclosed herein can be combined with one or more known presbyopia and/or refractive therapies. Although many embodiments are described with reference to a natural lens of the eye, the embodiments disclosed herein can be used to improve vision with IOLs.

The methods and apparatus disclosed herein are well suited for combination with other treatments such as surgical implants, intraocular lenses, laser in situ keratomileusis (LASIK), photorefractive keratectomy, small incision lens extraction (SMILE), crosslinking and corneal crosslinking.

PCT Application PCT/US20114/023763, filed on Mar. 11, 2014, incorporated herein by reference, discloses improved methods and apparatus to treat presbyopia and/or glaucoma in accordance with many embodiments disclosed herein. In many embodiments, tissue is not substantially removed and is moved to a new location with the treatment. This movement of collagenous tissue from a first location to a second location provides improved treatment with less regression of effect and healing. The methods and apparatus disclosed therein describe treatment of the eye without ablation and without formation of hard spots as can be formed when a laser removes tissue with heat. In many embodiments, the treatment can be performed without incisions of the eye, in order to decrease the invasiveness of the procedure and decrease regression of effect.

Examples of treatment modalities of the eye suitable for use with the systems and/or methods disclosed herein are described in PCT/US2014/023763, filed on 11 Mar. 2014, entitled “SCLERAL TRANSLOCATION ELASTO-MODULATION METHODS AND APPARATUS” (attorney docket no. 48848-703.601); and PCT/US2016/055829, filed on 6 Oct. 2016, entitled “ULTRASOUND DIRECTED CAVITATIONAL METHODS AND SYSTEMS FOR OCULAR TREATMENTS” (attorney docket no. 48848-704.601); the entire disclosures of which are incorporated herein by reference.

In many embodiments, the scleral translocation elasto-modulation (“STEM”) adjustment procedure provides application of heat to the eye to produce a thermo-mechanical response in a tissue of the eye, such as in the cornea and/or sclera. For example, the cornea and/or sclera can be heated to a range from about 50 to about 70 degrees Centigrade, for example between 60 and 70 degrees Centigrade, to produce shrinkage of the tissue. Tissue may be heated within the range without substantially weakening the tissue. In many embodiments, a portion of the eye can be heated to a temperature within a range of up to about 55 or 60 degrees Centigrade in order to relax the tissue. Heating of the cornea and/or sclera to a temperature within this range can also produce softening and/or plasticizing (e.g., to approximately 10% of the native strength of the tissue). The cornea and/or sclera can be heated to greater than 80 degrees Centigrade of the eye to produce denaturation of the tissue. The tissue may be weakened by heating to a temperature within a range from about 70 to about 90 degrees Centigrade.

The heating of tissue can be controlled in order to provide desired amounts of shrinkage or relaxation and combinations thereof. For example, heating collagenous tissue such as scleral tissue to a temperature within a range from about 50 to 70 degrees C. can result in shrinkage of the tissue that can be effective to move tissue to open Schelmm's canal for example. For temperatures in a range from about 60 to 70 degrees C., heating of the tissue can result in shrinkage or relaxation, depending on how long the tissue is heated. For example, heating tissue within a range from 60 to 70 degrees C. for shorter amounts of time can result in tissue shrinkage, while heating tissue for longer amounts of time can result in relaxation. To relax tissue, the tissue temperature can be elevated to a temperature in a range from about 60 degrees C. to about 80 degrees C. For example, heating tissue to about 80 degrees C. for about a millisecond can result in tissue relaxation. For lower temperatures within this 60 to 80 C range, the tissue can be heated for amounts of time longer than 1 ms to provide tissue relaxation.

The present inventors have determined with finite element analysis (FEA) analysis treatment regions suitable STEM adjustment. The treatment can be located in one or more of the cornea and sclera, for example slightly below the epithelium and conjunctiva. The treatment region can be located in scleral tissue and can be about 0.25 to about 0.75 mm deep, for example. The corneal treatment region can be from about 0.100 um to about 400 um deep, for example.

When light energy is used, the depth of tissue with sufficient heating can depend on the wavelengths of light energy. For example, light energy having wavelengths in a range from about 1.9 to 2.1 um, the 1/e attenuation depth can be in a range from about 200 to 300 ums, for example about 225 to 275 um. For light energy having wavelength in a range from about 1.3 to 1.6 um, the 1/e attenuation depth is within a range from about 350 to 450 um. When combined with the cooling lens as described herein, the profile of tissue heating can result in a peak temperature that is located beneath the exterior surface of the ocular tissue, even though the amount of light energy absorbed near the surface is greater than the amount of light energy absorbed at the tissue location which undergoes the highest amount of temperature increase. The depth of tissue that shrinks or relaxes can have a profile extending to a depth in the tissue.

In some instances, electro-sprayed water droplets may be used in combination with treatment with light energy in order to induce cavitation in the treated tissue. For example, water and/or heavy-water droplets may be electro-sprayed or mono-dispersed over a scleral treatment region without a corona discharge in ambient air prior to or during treatment of the tissue with light energy. Deposition of the micro-droplets may be electronically controlled and steered toward locations of interest. The droplets may for example comprise highly charged gas-encased water. The droplets may self-assemble due to the high charge carried in the sub-surface scleral volume. When seeded for micro-bubbles under laser irradiation, the droplets may migrate uniformly inside the irradiated tissue in order to create ordered pathways which may have enhanced stability and which may improve fluid outflow in the treated scleral region. Alternatively or in combination, cavitation of the tissue may open tight junctions which may improve permeation and stability without requiring the use of viscosity additives of penetration enhancers as have been previously used in the art. Such droplets may further be configured to carry cavitation seeding agents such as oxygen or carbon dioxide in order to enhance cavitation beyond that of the droplets alone. The droplets may alternatively or in combination be configured to carry other drugs or medicaments into the scleral tissue as desired by one of ordinary skill in the art.

The combination of light energy and micro-droplets may allow for the generation of scleral vacuoles (i.e. increased scleral porosity) with lower dose irradiation that would be possible with the use of light energy alone, which may be beneficial in at least some instances. Intra-stromal delivery may for example be achieved with 10 kV at 100 nA, which may allow the system to operate as a battery-operated unit. The combination of light energy and micro-droplets may allow for non-thermal cavitation of tissue which may be substantially similar to ultrasonic cavitation in its tissue effects as described herein.

The ultrasound methods and apparatus as disclosed herein can be used to heat tissue with similar temperatures and locations as described with reference to laser treatment, in order to provide glaucoma treatment as described herein. Other types of energy as described herein can be used similarly to treat glaucoma as will be appreciated by one of ordinary skill in the art. The ultrasonic approaches as described herein can also be used to soften tissue without substantial heating, for example.

In many embodiments, the methods and apparatus described herein can be used in combination with STEM to treat one or more of many disorders of the eye, and can be used to treat many of these disorders with an energy source, under control of computer instructions. The STEM apparatus can be used to one or more of soften or resect tissue with non-thermal treatments, for example less than about 50 degree Centigrade (degrees C.). Alternatively or in combination the methods and apparatus can be used in a thermal mode to treat tissue thermal with treatments more than about 50 degrees C., for example about 60 degrees C. or more. The non-thermal treatment can be used in many ways, such as for accurate tissue resection. Ultrasonic non-thermal tissue resection can be performed without substantial bubble formation, which allows the user such as a surgeon to accurately treat many regions of the, in many instances without interference from bubbles. In many instances, the mechanism of non-thermal treatment is substantially mechanical, such that tissue can be resected with very fine and accurate incision structures, which can be three dimensional. The STEM adjustment-induced cavitation can focally disrupt or liquefy or micro-porate (spongify) tissue and reduce rigidity, thus enhancing mobility of accommodative complexes and/or aqueous outflow facilities. By inducing cavitation non-thermally, the methods and system disclosed herein can provide improved safety over currently available thermal treatments.

Three or more types of treatments can be provided depending on energy delivery settings: 1. liquefaction, 2. paste or 3. vacuolated thermal treatment. Liquefied treatment regions are pure mechanically-disrupted treatment regions and can be observed for example with an ultrasound pulse duration less than 30 ms, which is slightly longer than time to boil. Paste treatments represent an intermediate state between mechanically-induced liquefaction and vacuolated thermal treatment. Paste treatment regions may be generated non-thermally, e.g. spongification, or thermally, as a pre-cursor state to vacuolated thermal treatment, or with a combination of both thermal and non-thermal settings. The use of chilled (4 degree C.) degassed Trehalose (optionally with NSAIDs) may be preferred over water as the coupling medium for improved ocular surface lubrication, in some embodiments.

The optional use of nanoparticles similar to nanoparticles for enhanced imaging can be used to enhance cavitation in some embodiments. Nanoparticles can be used with ultrasound treatment as disclosed herein to reduce the cavitational dosage requirements, for example by a factor of 2×-10×. The nanoparticles may comprise one or more of perfluorocarbon, lipid, albumin, or galactose, for example. Targeted (optionally drug-free) lysis due to microstreaming and micro-fragmentation (<5 um diameter) can improve micro-circulation and contains region of treatment demarcation with added safety. Treatments can be provided with decreased bleeding and decreased apoptosis, which can be shown with blood brain barrier and myocardial infraction studies, for example. While the nanoparticles can be used for any of the treatments disclosed herein such as glaucoma treatments, the nanoparticles can be beneficial for fractionation and apoptosis of choroidal neovascularization (CNV) and uveal melanomas, for example.

Drug delivery can be enhanced with the methods and apparatus disclosed herein. Debulking of tissue as disclosed herein can be used as a preparatory step and may be advantageously administered in combination drug delivery to promote drug delivery and improve delivery of the drug through the treated tissue.

The controlled cavitation as described herein can be provided with simultaneous imaging administered from the STEM apparatus to the tissue with a fluidic coupling path and a rotating arm to sequence the focused patterns within the tissue to deliver the drug at doses related to the scan rates of the ultrasound beam as disclosed herein.

The tissue can be treated so as to provide small zones from which the tissue is removed by natural processes such as macrophages in order to remove tissue. The removed tissue can be removed from several small locations so as to make the tissue more elastic, similar to a sponge.

The energy can be delivered so as to generate cavitation and increase elasticity of the target tissue with decreased amounts of heat. In many instances, the STEM adjustment treatment provides debulking of the tissue which increases the elasticity of the tissue. The amount of heating of the treated tissue can be controlled to be no more than about 10 degrees C., for example no more than about 5 degrees C., which can increase elasticity with decreased amounts of regression.

Light Energy Sources

The STEM adjustment system may comprise an energy delivery system configured to delivery energy to the eye. The tissue may for example comprise the sclera or the cornea. One or more of the energy source, processor, or energy delivery system may be configured to deliver energy to the eye.

In many embodiments, the STEM adjustment procedure provides extra-corneal and/or extra-lenticular energy treatment to soften and/or plasticize the sclera and/or peripheral cornea, such as with one or more of light energy, ultrasound energy, high intensity ultrasound energy, mechanical energy, radiofrequency energy, electrical energy, thermal energy, electroporation, microwave energy, optoporation, photonic desincrustation, or galvanic desincrustation.

While reference is made to softening tissue with light energy, it will be apparent to one of ordinary skill in the art that other forms of energy can be used to soften tissue such as one or more of ultrasound energy, electroporation, microwave, thermal, electrical energy an electrophoretic energy source, or di-electrophoretic energy and combinations thereof. In many embodiments, electroporation needles can be provided with a shaped array having four quadrants sized to extend through the conjunctiva and deliver electroporation energy beneath the conjunctiva. Alternatively, shaped contact electrodes can be provided without needles such that the current is passed through the epithelial layer of the conjunctiva to targeted regions of the sclera. The electroporation to soften the sclera comprises an oscillating electric field to pass current in an electroporation treatment profile similar to the optical treatment profile disclosed herein.

The energy can be delivered with an optical or ultrasound delivery system, and the system may comprise an energy delivery system to direct energy to the eye or a hand held probe, and combinations thereof, for example.

In many embodiments, light energy is used to soften the tissue, and the light energy comprises wavelengths that are strongly absorbed by the collagen of the sclera or the water of the sclera, or both for example. In many embodiments, the light energy comprises wavelengths that are absorbed more strongly by stromal tissue than water, for example light comprising a wavelength within a range from about 4 to 6 um, such as from about 5.5 to 6.6 um. The light energy absorbed more strongly by stroma than water has the advantage of providing more accurate treatment with less interference with water, and can allow the tissues of the eye to retain healthy amounts of water during treatment, for example tissues of the conjunctiva of the eye. Also, interference from water based surgical fluids such as saline and anesthetics can be substantially inhibited.

In many embodiments, the light energy comprises wavelengths within a range from about 1 to 6 um, such as from about 1 to 3 um. In many embodiments the light energy comprises wavelengths within a range from about 1.4 to about 2 um, for example about 1.46 um or 2.01 um, and other ranges as described herein.

The laser may comprise one or more of many lasers emitting one or more of many wavelengths, such as infrared lasers. In many embodiments, the laser comprises a quantum cascade laser configured to emit light having a wavelength within a range from about 5.8 to about 6.6 um, for example from about 6 to about 6.25 um. In many embodiments, the laser comprises a quantum cascade laser or continuous wave laser configured to emit light having a wavelength within a range from about 1 to about 6 um, such as from about 1 to 3 um. In many embodiments the laser is configured to emit light having a wavelength within a range from about 1.4 to about 2 um, for example about 1.46 um or 2.01 um, and other wavelength ranges as described herein. Such lasers are commercially available, and can be configured by a person of ordinary skill in the art for treatment of the eye as described herein.

Heat Sink

In many embodiments a heat sink is provided to couple to the conjunctiva and the heat sink comprises a material transmissive to the light energy, such as sapphire or Zinc Selenide (hereinafter “ZnSe”). The heat sink material can be configured to transmit light energy absorbed more strongly by the stroma than water, and may comprise Zinc Selenide (hereinafter “ZnSe”), for example. The heat sink can be chilled to inhibit damage to the conjunctiva of the eye. The heat sink can provide improved transmission of light energy when condensation is present, as the condensed water may be less strongly absorbed by the laser beam. In many embodiments, one or more layers of the epithelium of the eye (basal layer, wing layer or squamous layer) remains substantially intact above the zone where the eye has been treated, for example at least one layer of viable epithelial cells can remain intact when the heat sink is removed.

In many embodiments, the optically transmissive material of the heat sink is shaped and optically configured with smooth surfaces so as to comprise an optically transparent heat sink such as a lens. The heat sink may comprise a window of the optically transmissive material, and can be one or more of many shapes such as a flat on opposing surfaces, plano-concave, or convex-concave. The convex-concave heat sink window may comprise a meniscus shaped lens, having substantial optical power or no substantial optical power, for example.

The location of the heat sink can be fixed in relation to a fixed structure of the laser system in order to fix the location of the eye, and the heat sink may comprise one or more curved surfaces such as a concave surface to engage the eye. In many embodiments, an arm extends from the fixed structure of the laser system to the heat sink in order to fix the location of the heat sink.

In many embodiments the STEM adjustment treatment apparatus comprises an energy source such as a laser and a docking station to retain the eye. In many embodiments the docking station comprises a chilled optically-transmissive heat sink to couple to the eye. The docking station couples to the eye such that the heat sink contacts the conjunctiva of the eye and fixes the working distance of the eye relative to the surgical laser, such that the scleral treatment comprising softening posterior to the lens equator can be performed accurately. In many embodiments, the heat sink is chilled such that at least one epithelial layer of the conjunctiva of the eye above the treated tissue remains viable, in order to expedite healing of the eye and decrease invasiveness of the procedure. The chilled heat sink structure can be chilled to a temperature within a range from above the freezing temperature of the eye and saline, at about −3 degrees Celsius (C), to below an ambient room temperature of about 20 degrees Celsius. Alternatively, a heat sink can be provided without chilling. In many embodiments, the freezing temperature of the eye corresponds to the freezing temperature of saline, about −3 degrees, for example. In many embodiments, the apparatus comprises a scanner to scan the laser beam. The laser beam can be pulsed or continuous, and in many embodiments comprises a continuous laser beam configured to inhibit temperature spikes related to ablation of the eye. In many embodiments the laser irradiance comprises a temporal and spatial profile to inhibit transient heating peaks of the tissue that can be related to removal of tissue such as ablation. The scanner can be configured to scan the laser beam in a plurality of quadrants, such as for quadrants with untreated regions between each of the quadrants to inhibit damage to muscles of the eye located between the treatment quadrants.

The cooling methods and apparatus disclosed herein can be combined with the energy sources described herein in order to decrease heating of tissue near external surfaces of the eye, such as conjunctival and epithelial layers of the eye. Decreased heating of tissue near external surfaces of the eye may result in the tissue near the external surfaces of the eye remaining substantially viable when the tissue below it is treated. This may for example be done in order to inhibit pain and swelling of the eye during and/or after treatment.

Ultrasound Energy Sources

In some embodiments, the energy source may be an ultrasound energy source, for example an ultrasound phased array. The ultrasound phased array can be programmed to treat non-adjacent focal zones with a very high duty cycle, e.g. greater than about 50% from the phased array, while each of focal treatment zones has a duty cycle less than about 5%, for example 2.5% or less in order to provide non-thermal tissue resection with very high pulse repetition frequencies in order to decrease treatment time. The phased array can also be used for imaging the tissue during treatment with imaging ultrasound from the array.

Due to the benefits of sub-surface tissue debulking/softening, high frequency histotripsy transducers, such as preferably electronically steerable phased array 5 MHz-20 MHz HIFU transducers can be used at under 250 W and with pulses within a range from 100 nsecs to 100 msec pulses. The pulse frequency can be under 1000 Hz repetition rates for sequential and non-sequential ocular treatments as described herein.

Using a customized deposition nomogram, temperature outside of the histotripsy focal zone may not exceed 50 degrees C. thus protecting tissue at depth. Negative acoustic pressures of up to −80 MPa (typically −30 MPa) can be provided and can be sufficient to provide a 10% debulking rate for a 360 degree treatment 3 minutes long.

The methods and system disclosed herein can provide an energy source such as a high intensity focused ultrasound (HIFU) energy source for treatment of tissue so as to increase elasticity of the tissue. The methods and system disclosed herein may utilize HIFU treatment to induce cavitation in a non-incisional and non-thermal manner. HIFU tissue penetration is not dependent on the opacity of the tissue, therefore HIFU may have greater access to tissue than laser systems which cannot penetrate through opaque media. Additionally, by inducing cavitation non-thermally with HIFU, the methods and system disclosed herein may prevent boiling bubble formation during cavitation and subsequent opacification of treated tissue.

The increased elasticity of the tissue can be provided at locations arranged in order to provide a therapeutic effect, such as glaucoma treatment, with decreased amounts of regression. In many embodiments, the ultrasound beam can be focused to a small spot size with a frequency within a range from about 5 to 25 MHz (mega Hertz) in order to provide improved accuracy as shallow locations such as 1 mm or less below a surface of the eye, for example within a range from about 0.1 to about 0.9 mm.

The methods and system disclosed herein can provide a focused spot having a cross-sectional size within a range from about 50 um to about 200 um full width half maximum (FWHM); the corresponding cavitation can be similarly sized within similar ranges. The energy beam, for example an ultrasound beam, can be focused and pulsed at each of a plurality of locations to provide a plurality of cavitation zones at each of the target regions. Each pulse may comprise a peak power within a range generating focal negative peak pressures of about 30 MPa (mega Pascals). While the treatment pulses can be arranged in many ways within a region, in many instances the pulses can be spaced apart within a region to provide intact tissue such as intact sclera between pulses. Alternatively or in combination, the pulses can be overlapped to provide an overlapping treatment region having dimensions within a range from about 100 um to about 1 mm, and a plurality of spaced apart treatment regions can be provided within a treatment location. The depth of the treatment can be controlled in accordance with the region being treated. For example, glaucoma treatments of Schlemm's canal can be about 0.5 mm or less, and treatment regions of the ora serrata which can be deeper, for example within a range from about 0.5 to about 1.0 mm deep. For treatments located along the ciliary apex the treatment can be within a range from about 0.25 mm to about 0.75 mm.

The treatment geometry can be arranged in many ways and may comprise a length within a range from 100 um to 1 mm, a volumetric region within a range from (400 um×100 um×360 degrees) and durations of exposure of less than 3 minutes are easily managed with a motorized circumferential track and 5 MHz-10 MHz theranostic applicator.

Use of dual frequency ultrasound with a low frequency pump combined with high frequency ultrasound can be used to reduce the high frequency cavitation threshold.

The methods and apparatus can be configured in many ways to treat tissue. This system can be configured to generate one or more of liquefaction, or vacuolated tissue, for example. The STEM adjustment system can be configured to provide mechanical erosion of collagen with breaking of the collagen fibers with well-defined margins using an energy source, for example one or more of light energy, ultrasound energy, radiofrequency energy, electrical energy, thermal energy, electroporation, microwave energy, optoporation, photonic desincrustation, or galvanic desincrustation.

The following boiling histotripsy parameters and responses describe an upper treatment limit using ultrasound energy in accordance with examples. The treatment energy can be substantially lower than shown in Table 1 below. Alternatively, treatment parameters similar to those shown in Table 1 can be used with decreased amounts of time.

TABLE 1
Boiling Histotripsy parameters and histopathology responses

| | Pulse | Duty | | Protein | Border Destroyed | Effect Within |
|---|---|---|---|---|---|---|
| | Duration | Factor | PRF | Denaturation | vs Intact | Lesion |
| Liquefied | <30 ms | <.02 | 1 Hz | 0% | <40 um | mechanical |
| Paste | <100 ms | <0.2 | <2 Hz | 22%-27% | <40 um | mechanical & |
| | | | | | | thermal |
| Vacuolated | >100 ms | >0.2 | >2 Hz | 70%-90% | <100 um | thermal |

The STEM adjustment system may be operated in mechanical mode to produce purely mechanical effects or in thermal mode to produce thermal effects in the tissue when exposed to an energy source such as HIFU. Mechanical mode using HIFU comprises a duty cycle of less than 2.5%, more preferably less than 1%. Thermal mode comprises a duty cycle of more than 2.5%. The device may be operated in either mechanical mode or thermal mode and may be readily switched between the two modes.

HIFU may be operated with a duty cycle range of about 0.1% to about 1% for cavitational histotripsy or a duty cycle range of about 1% to about 2.5% for boiling histotripsy depending on the energy applied by the HIFU STEM system described herein. HIFU may be operated with a duty cycle range of about 0.01% to about 1% for cavitational histotripsy or a duty cycle range of about 1% to about 2.5% for boiling histotripsy depending on the energy applied by the HIFU STEM system described herein.

The methods and system described herein may be operated with any combination of the parameters listed in Table 2.

TABLE 2
Treatment parameters.

| Parameter | Range | Preferred |
|---|---|---|
| HIFU frequency | 750 kHz to 25 MHz | 10 MHz |
| Total treatment duration | 0 min to 10 min | 4 min |
| PRF | 1 Hz to 1000 Hz | 1000 Hz |
| Non-thermal duty cycle | 0.1% to 2.5% | 1% |
| Negative acoustic | −10 MPa to −80 MPa | −30 MPa |
| pressure | | |
| Tissue temperature | 37° C. to 100° C. | 41° C. |
| Treatment size | 100 um × 400 um | May be configured |
| | (per focal point) | to scan and treat |
| | | multiple regions |
| | | with focal points |
| Treatment depth | 0 cm to 2.5 cm | 1 cm |
| Focal gain | 10 to 100 | Typical: 50 |

The STEM adjustment system may be operated at a HIFU frequency within a range of about 750 kHz to about 25 MHz, for example within a range of about 1 MHz to about 25 MHz, preferably within a range of about 5 MHz to 15 MHz, more preferably within a range of about 5 MHz to 10 MHz, more preferably about 10 MHz. The HIFU frequency for example may be within a range of about 2 MHz to about 24 MHz, for example within a range of about 3 MHz to about 23 MHz or within a range of about 4 MHz to about 22 MHz. The frequency for example may be within a range of about 5 MHz to about 21 MHz, within a range of about 6 MHz to about 20 MHz, or within a range of about 7 MHz to about 19 MHz. The frequency may for example be within a range of about 8 MHz to about 18 MHz, within a range of about 9 MHz to about 17 MHz, or within a range of about 10 MHz to about 16 MHz. The frequency may for example be within a range of about 11 MHz to about 15 MHz, within a range of about 12 MHz to about 14 MHz, or within a range of about 10 MHz to about 13 MHz.

The total STEM adjustment treatment duration may be up to 10 minutes, for example within a range from about 1 min to about 10 min, preferably about 4 min. The total treatment duration may for example be within a range of about 2 min to about 9 min, within a range of about 3 min to about 8 min, or within a range of about 4 min to about 7 min. The total treatment duration may for example be within a range of about 5 min to about 6 min. The total treatment duration may for example be within a range of about 2 min to about 6 min, preferably within a range of about 3 min to about 5 min, or within a range of about 4 min to about 6 min, and more preferably within a range of about 4 min to about 5 min. The total treatment duration for example may be within a range of about 3 min to about 10 min, or within a range of about 4 min to about 8 min.

The PRF of the HIFU energy source of the STEM adjustment system described herein may be within a range of about 1 Hz to about 1000 Hz, for example within a range of about 50 Hz to about 1000 Hz, preferably about 1000 Hz. The PRF may for example be within a range of about 100 Hz to about 900 Hz, within a range of about 200 Hz to about 800 Hz, or within a range of about 300 Hz to about 700 Hz. The PRF for example may be within a range of about 400 Hz to about 600 Hz, for example about 500 Hz to about 600 Hz. The PRF for example may be within a range of about 100 Hz to about 1000 Hz, preferably within a range of about 200 Hz to about 1000 Hz, more preferably within a range of about 500 Hz to about 1000 Hz.

The non-thermal duty cycle of the HIFU energy source of the STEM adjustment system described herein may be within a range of about 0.1% to about 2.5%, preferably less than 1.0%. The non-thermal duty cycle may for example be within a range of about 0.2% to about 2.4%, within a range of about 0.3% to about 2.3%, or within a range of about 0.4% to about 2.2%. The non-thermal duty cycle may for example be within a range of about 0.5% to about 2.1%, within a range of about 0.6% to about 2.0%, or within a range of about 0.7% to about 1.9%. The non-thermal duty cycle may for example be within a range of about 0.8% to about 1.8%, within a range of about 0.9% to about 1.7%, or within a range of about 1.0% to about 1.6%. The non-thermal duty cycle may for example be within a range of about 1.1% to about 1.5%, within a range of about 1.2% to about 1.4%, or within a range of about 1.2% to about 1.3%. The non-thermal duty cycle may for example be within a range of about 0.5% to about 1.5%, preferably within a range of about 0.7% to about 1.3%, more preferably within a range of about 0.8% to about 1.2%.

The number of cycles of the STEM adjustment system described herein may be within a range of about 1 to about 100 cycles, for example about 10 to about 100 cycles. The number of cycles may be within a range of about 20 to about 100 cycles, for example about 30 to about 100 cycles, for example about 40 to about 100 cycles. The number of cycles may be within a range of about 50 to about 100 cycles, for example about 60 to about 100 cycles, for example about 70 to about 100 cycles. The number of cycles may be within a range of about 80 to about 100 cycles, for example about 90 to about 100 cycles. The number of cycles may be within a range of about 10 to about 50 cycles, for example about 10 to about 30 cycles. The number of cycles may be within a range of about 10 to about 80 cycles, for example about 20 to about 50 cycles.

The peak negative acoustic pressure of the HIFU energy source of the STEM adjustment system described herein may be within a range of about −10 MPa to about −80 MPa, preferably about −30 MPa. The negative acoustic pressure may for example be within a range of about −20 MPa to about −70 MPa, within a range of about −30 MPa to about −60 MPa, or within a range of about −40 MPa to about −50 MPa. The negative acoustic pressure may for example be within a range of about −10 MPa to about −50 MPa, preferably within a range of about −20 MPa to about −40 MPa, more preferably about −30 MPa.

The negative acoustic pressure of the HIFU energy generated at the cornea by the STEM adjustment system may for example be calculated using the formula:

$${P}_{C} = {P}_{F} \frac{{A}_{F}}{{A}_{C}} \qquad ( 1 )$$

Where P_{C}=pressure at the cornea, P_{F}=pressure at the focal point of the HIFU energy, A_{F}=area of the focal point, and A_{C}=area of the cornea in line of the HIFU energy beam. The diameter of the focal point may for example be in a range from about 50 μm to 200 μm, thus the area of the focal point may be calculated to be about 1964 μm² to about 31416 μm². The negative pressure at the focal point may for example be in a range from about −10 MPa to about −80 MPa. The diameter of the cornea in the line of the HIFU beam may for example be about 3 mm, thus the area of the cornea may be about 7.07 mm². Using formula 1 to calculate the pressure at the cornea given the exemplary ranges described, the negative acoustic pressure at the cornea may for example be within a range of about 2.8 kPa to about 356 kPa.

The negative acoustic pressure at the cornea may for example be within a range of about 1 kPa to about 350 kPa, for example within a range of about 1 kPa to about 300 kPa. The negative acoustic pressure at the cornea may for example be within a range of about 1 kPa to about 250 kPa, for example about 1 kPa to about 200 kPa. The negative acoustic pressure at the cornea may for example be within a range of about 0 kPa to about 150 kPa, for example about 1 kPa to about 100 kPa. The negative acoustic pressure at the cornea may for example be within a range of about 1 kPa to about 50 kPa, for example about 1 kPa to about 10 kPa.

The temperature of the tissue treated with the STEM adjustment system may be within a range of about 37° C. to about 100° C., preferably 41° C. The temperature of the tissue may for example be within a range of about 37° C. to about 50° C., preferably within a range of about 37° C. to about 45° C., more preferably within a range of about 37° C. to about 44° C., still more preferably within a range of about 37° C. to about 41° C.

The treatment size per focal point may be about 100 um×400 um. The STEM adjustment system described herein may be configured to scan and treat multiple regions with multiple focal points, thus the total treatment area may be any area of any size within the eye.

The treatment depth of the STEM adjustment system described herein may be within a range of about 0 cm at the surface of the eye to about 2.5 cm deep within the eye, preferably about 1 cm depending on the target tissue. The treatment depth may for example be within a range of about 0.1 cm to about 2.4 cm, within a range of about 0.2 cm to about 2.3 cm, or within a range of about 0.3 cm to about 2.2 cm. The treatment depth may for example be within a range of about 0.4 cm to about 2.1 cm, within a range of about 0.5 cm to about 2.0 cm, or within a range of about 0.6 cm to about 1.9 cm. The treatment depth may for example be within a range of about 0.7 cm to about 1.8 cm, within a range of about 0.8 cm to about 1.7 cm, or within a range of about 0.9 cm to about 1.6 cm. The treatment depth may for example be within a range of about 1.0 cm to about 1.5 cm, within a range of about 1.1 cm to about 1.4 cm, or within a range of about 1.2 cm to about 1.3 cm. The treatment depth may for example be within a range of about 0.25 cm to 0.75 cm, within a range of about 0.5 cm to about 1.5 cm, or 0.5 cm or less. The treatment depth is determined by the location of the tissue being treated.

The focal gain of the STEM adjustment system described herein may be within a range of about 10 to 100, for example within a range of about 20 to 90. The focal gain may for example be within a range of about 30 to 80, within a range of about 40 to 70, or within a range of about 50 to 60.

The voltage of the HIFU energy source of the STEM adjustment system described herein may be within a range of about 100V to about 400V, for example about 150V to about 350V. The voltage of the HIFU energy source of the STEM adjustment system described herein may be within a range of about 200V to about 300V, for example about 200V to about 250V.

Glaucoma

The systems and methods described herein are well-suited to treatment of glaucoma. Any type of glaucoma may be treated by restoring outflow through adjustment of one or more of the three primary outflow pathways. Treatment may be done so as to open a narrow angle such as a closed angle, dilate and/or stretch the Trabecular Meshwork and/or Schlemm's Canal, increase porosity and/or dilate vacuoles of the perilimbic sclera, or any combination thereof. The glaucoma treatment method may include 360 degree treatment in an annular pattern. Two annuli may be patterned to straddle the scleral spur, for example from 1 mm outside of the maximum to 1 mm inside the minimum of the limbus, so as to treat approximately above the roof of Schlemm's Canal.

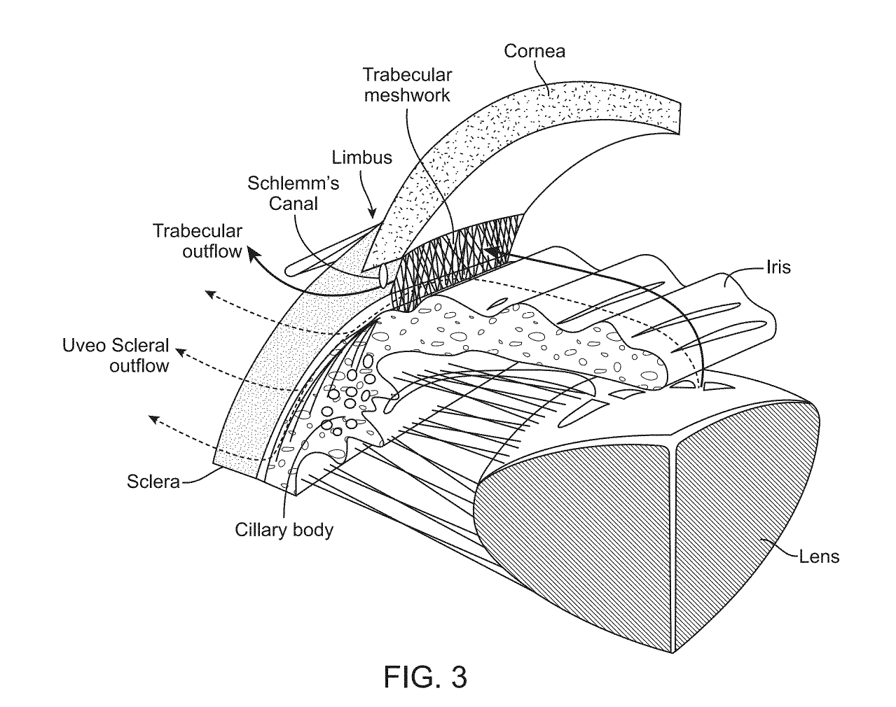

FIGS. 3-50D show glaucoma treatment methods and apparatus as will be understood by a person of ordinary skill in the art. The methods and apparatus as described herein can be combined in many ways to treat glaucoma, for example with reference to PCT/US2017/023092, the entire disclosure of which is hereby incorporated by reference, which may be combined with FIGS. 3-50D in accordance with embodiments disclosed herein. A single laser system can be configured for both glaucoma treatment and treatment of refractive error for example.

The STEM systems as described herein can be configured to treat glaucoma, for example with ultrasound, electromagnetic energy, or other forms of energy, and combinations thereof. The processor can be configured with instructions to apply energy with patterns, amounts, intensities, and durations as described herein in order to treat glaucoma. The energy source and instructions can be configured to apply a generally annular pattern of energy to the eye near Schlemm's canal, for example. The generally annular pattern can be aligned to the eye with reference to the limbus, which is located at the corneal/scleral junction near Schlemm's canal. The location of Schlemm's canal with respect to the limbus may vary systematically with age and/or IOP. For example, Schlemm's canal may be further away from the limbus in younger eyes than in older eyes. Schlemm's canal may be further away from the limbus in patients with increased IOP compared to patients with normal IOP. Such variations may be taken into account when patterning treatment. For example, treatment may be patterned further out from the limbus in an older patient than in a younger patient to account for the difference in location of Schlemm's canal with reference to the limbus.

While the generally annular pattern can be configured in many ways, the annular pattern may comprise an outer ring (or segments of a ring, e.g. dots along a ring) and an inner ring (or segments of a ring, e.g. dots along a ring), such that the limbus is located therebetween when viewed axially from a location anterior to the eye such as with an operating microscope. The inner ring can be located radially inward about 1 mm from Schlemm's canal and the outer ring located radially outward about 1 mm from Schlemm's canal. The inner ring can be located radially inward about 2 mm from Schlemm's canal and the outer ring located radially outward about 2 mm from Schlemm's canal. The inner ring can be located radially inward about 3 mm from Schlemm's canal and the outer ring located radially outward about 3 mm from Schlemm's canal. Alternatively, the generally annular pattern may comprise an annulus having inner and outer dimensions similar to the first ring and the second ring. Without being bound by any particular theory, the first ring separated from the second ring can induce improved stretching between the rings similar to a suspension effect and may be related to improved dilation and porosity.

The contact lens, heat sink, and/or cooling structure as described herein can be used to conduct heat to reduce heating, for example when the energy source comprises a light source such as a laser as described herein, in order to leave the epithelium substantially intact. The energy source can be applied at locations in order to shrink tissue near Schlemm's canal and provide dilation of Schlemm's canal, the trabecular meshwork, or the collector channels, and combinations thereof in order to increase aqueous outflow and reduce intraocular pressure. Alternatively or in combination, the energy source can be configured to treat scleral tissue so as to create vacuoles in the sclera, so as to reduce IOP. The combination of dilation of Schlemm's canal, the trabecular meshwork, collector channels, and scleral vacuoles are believed to be particularly advantageous to the treatment of glaucoma.

Scleral vacuoles can be formed by treating scleral tissue with treatment parameters as described herein. The scleral tissue may be treated with a generally annular pattern, for example a plurality of spaced apart rings in order to create or expand vacuoles for improved outflow through the sclera. Alternatively, the annular treatment pattern may comprise an annulus, or potion thereof, for example. The annular treatment pattern may comprise a plurality of overlapping rings or spots from individual laser pulses, for example.

The STEM glaucoma treatment energy may comprise laser or ultrasound energy as described herein, for example, although other forms of energy can be used such as radiofrequency energy.

The systems and methods described herein may be used to perform scleral translocation elasto-modulation (“STEM”). The systems and methods described herein may include STEM-mediated Schlemm's canal adjustment for glaucoma treatment. The systems and methods described herein may be used to treat all types of glaucoma.

In many embodiments, the eye includes a conjunctiva disposed over a sclera and an inner portion of the eye (e.g. sclera) located below the conjunctiva is treated through the conjunctiva of the eye. The eye can include a conjunctiva and the conjunctiva can be moved away from the sclera to treat an inner portion of the eye (e.g. sclera) located below the conjunctiva.

Imaging while Treating

The methods and system disclosed herein can be used in many ways and can be used to image the tissue during treatment. The STEM adjustment treatment system may comprise in imaging apparatus such that the treatment can be combined imaging with one or more of magnetic resonance (MR) imaging, ultrasound biomicroscopy (UBM), ultrasound (US) imaging, optical coherence tomography (OCT), optical coherence elastography (OCE), or US elastography transducer measurements. The imaging apparatus can be combined with the STEM adjustment treatment with either simultaneous oblique trans-iridional imaging or on the coaxial therapeutic probe; and diagnostic images that are useful intra-operatively, for visualization as well as for feature/landmark tracking. Rapid real time MR images can be acquired when time-synchronized to STEM adjustment energy pulses with weighting motion gradients turned ON for greater cavitational sensitivity. MR/OCT/US guided STEM adjustment guidance can include one or more of pretreatment planning, image-based alignment and siting of the STEM energy source focus, real-time monitoring of treatment energy-tissue interactions, or real-time control of exposure and damage assessment.

The STEM adjustment treatment system may comprise an imaging apparatus capable of determining tissue elasticity before, during, or after STEM adjustment treatment, or some combination thereof, for example OCE or US elastography transducers. The treatment system may additionally or in combination comprise a mechanism for real-time temperature sensing, for example using an OCT transducer, in order for real-time monitoring of laser- or HIFU-induced temperature changes or to provide for control of laser or HIFU exposure, respectively, to maintain temperature.

Motorized diagnostic imaging in sync with histotripsy patterning can be achieved in these configurations. For example, real-time imaging of treatment tissue may allow for user input to a grid of target regions, which may be larger than the area covered by a single treatment or include multiple areas not in direct contact with each other, for motorized control of multiple treatments over a larger area, allowing the user to avoid manual repositioning which may save time and prevent mistakes.

Imaging may be configured to occur simultaneously with treatment. A processor can be coupled to the ultrasound array and configured with instructions to scan the beam to a plurality of locations and image the tissue during treatment. The system may also comprise a display coupled to the processor that allows the user to see the tissue treated on the display and to plan the treatment. The images shown on the display can be provided in real time and can allow the operator to accurately align the tissue with the treatment and may allow the operator to visualize the treatment area, and other locations away from the treatment area. The imaging of the treatment area can be used to determine identify the target area on the screen and to program the treatment depth and location in response to the images shown on the display. The imaging can be used to visualize movement of ocular structures during treatment in order to detect beneficial treatment effects. The processor can be configured with instructions to treat the eye with a first wavelength of ultrasound and to image the eye with a second wavelength longer than the first wavelength. The processor may alternatively or in combination be configured with instructions to treat the eye with HIFU or laser energy and to image the eye with an embedded imaging apparatus, for example an optical coherence tomography (OCT) probe. The processor coupled to the array can be configured with instructions to provide both ultrasound wavelengths from the array. The imaging apparatus may provide additional tissue feedback data in real-time, for example temperature or elasticity.

The processor may be configured with instructions to determine one or more locations of the limbus, and/or one or more locations of Schlemm's canal. In response to the determined location of limbus, for example, one or more locations of Schlemm's canal may be determined. The processor may be configured with instructions to determine a treatment pattern based on the one or more locations of the limbus and/or the one or more locations of Schlemm's canal. The treatment pattern may for example comprise a juxtacanalicular treatment pattern. The processor may be configured to deliver shrinkage energy to the sclera, cornea, or both in order to urge tissue near Schlemm's canal to move towards the treated tissue and dilate Schlemm's canal as described herein.

Advantages of STEM Adjustment Procedure

The STEM adjustment procedure may provide one or more of the following advantages:

Increased depth of field of the eye;

Preservation of distance visual acuity, as the central corneal and central lenticular regions are substantially unaffected by the treatment;

Preservation of limbal stem cells, ciliary muscle function, conjunctiva, epithelium, and aqueous production, as these are substantially unaffected by treatment;

No substantial loss of contrast sensitivity;

No substantial disturbances of night vision;

Preservation of aesthetics of the eye, as there are no cuts, implants, or full punctures of the eye;

Rapid patient recovery, as the conjunctiva is protected during treatment;

Tolerable treatment procedure for many patients;

Improved safety of the treatment procedure;

Avoidance of dry eye;

Sparing of cornea tissue from damage;

Non-subtractive treatment of corneas allowing for treatment of thin corneas;

Smoothing of corneal striae (e.g. SRI);

Smoothing of capsular striae (e.g. cSRI);

Circularization of pupil;

Centering of lenticular sub-luxation;

Correction of lenticular astigmatism;

Correction of mild to moderate refractive errors (e.g. myopia, hyperopia, astigmatism);

Treatment of keratoconus (KCN);

Little additional optical power required, resulting in substantially no cross blurring; or

Other surgeries, including additional STEM adjustment treatments, are not precluded.

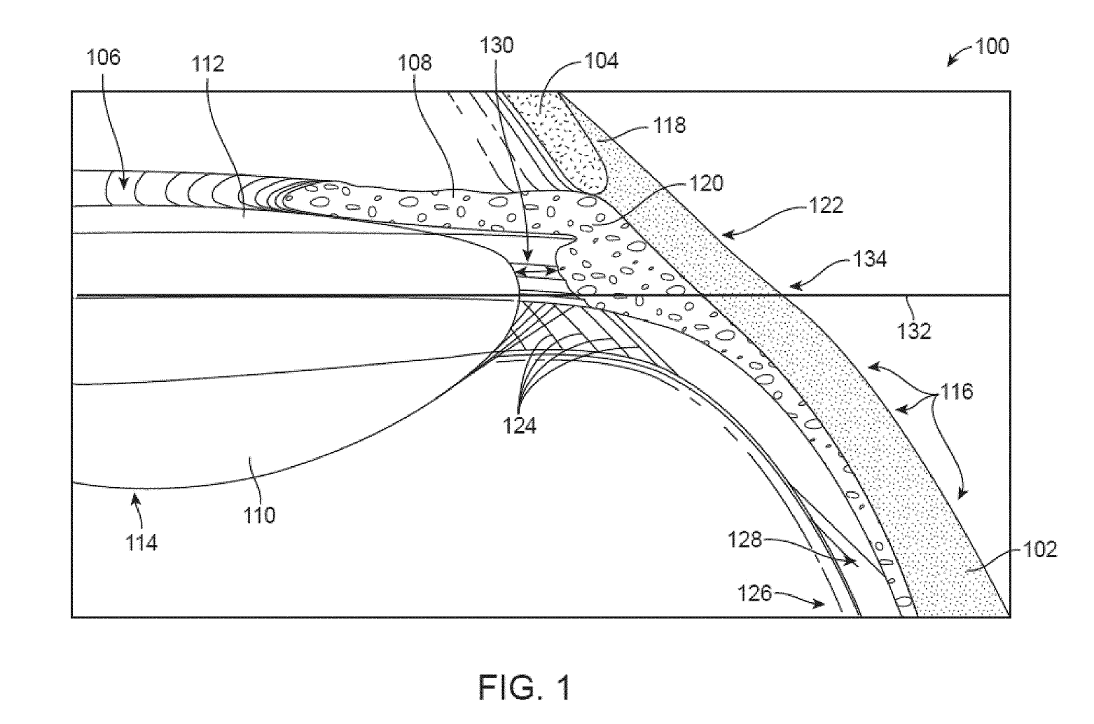

FIG. 1 illustrates an eye 100, in accordance with embodiments. The eye 100 includes a sclera 102, a cornea 104, a pupil 106, an iris 108, and a lens 110 within a lens capsule, the lens capsule including a lens capsule anterior surface 112 and a lens capsule posterior surface 114. The sclera is lined by a conjunctiva 116 and includes a sclera spur 118 adjacent the cornea 104. A ciliary body 120 is adjacent the ciliary body sclera region 122. The ciliary body 120 is connected to the lens 110 by vitreal zonules 124 and to the ora serrata 127 by the posterior vitreal zonules 128 (hereinafter “PVZ”). A circumlental space 130 (hereinafter “CLS”) is defined by the distance between the lens 110 and the ciliary body 120 along a lens equator plane 132, the lens equator plane 132 passing through an equatorial sclera region 134.

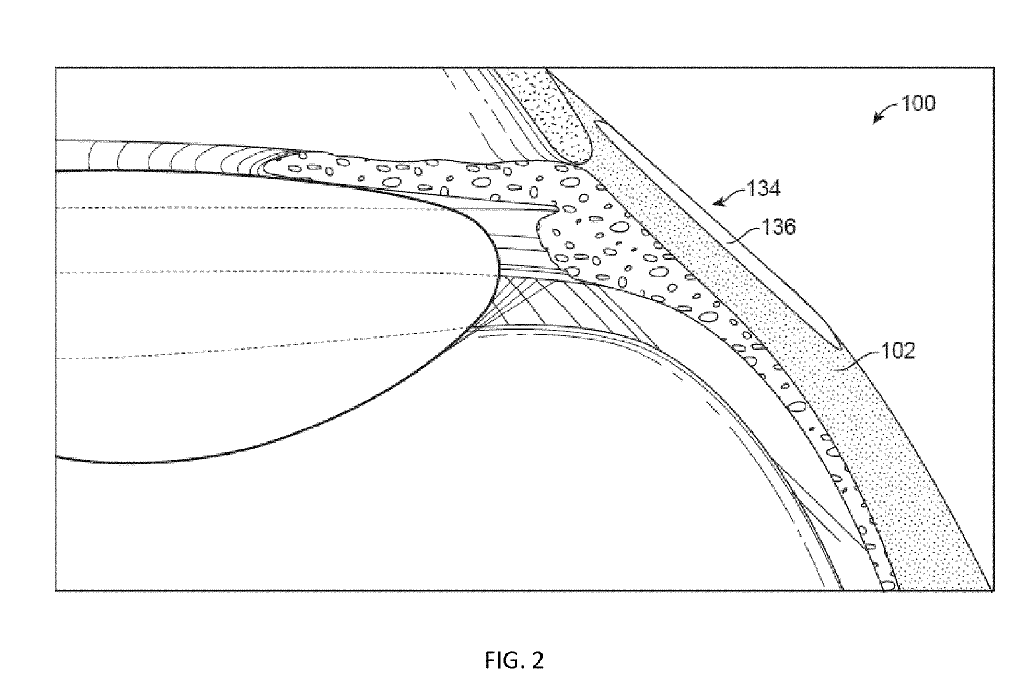

FIG. 2 illustrates stabilization of an eye 100 by cross-linking, in accordance with embodiments. The stabilized region 136 can be disposed in the outer portion of equatorial sclera region 134 of the sclera 102. Any suitable stabilization method, such as collagen cross-linking, can be used to stabilize the cross-linked region 136 in order to substantially maintain the outer profile of the sclera 102. In many embodiments, a cross-linking agent is applied to the sclera and allowed to infuse into the sclera at stabilized region 136. An energy source can be applied to the sclera to cross-link the sclera at stabilized region 136 with the cross-linking agent. The energy source can include a microelectrode array to generate a patterned cross-linked profile on the sclera. The energy can include one or more of thermal energy, radiofrequency (hereinafter “RF”) energy, electrical energy, microwave energy, light energy, or ultrasound energy.

In many embodiments, the cross-linking agent includes one or more of many known chemical photosensitizers in the presence of oxygen. Oxygen can be delivered to the stabilized region 136 of the sclera, concurrently with the cross-linking agent or separately. The cross-linking agent can be exposed to light energy when the cross-linking agent has been provided to the tissue, in order to provide cross-linking to a target depth of tissue stabilization. The light energy may include one or more of visible light energy, ultraviolet (hereinafter “UV”) light energy, or infrared (hereinafter “IR”) light energy. Alternatively or combination, the cross-linking agent may include a chemical cross-linking agent. In many embodiments, the cross-linking agent includes one or more of the following: riboflavin, rose bengal, methylene blue, indocyanine green, genipin, threose, methylglyoxal, glyceraldehydes, aliphatic β-nitro alcohols, black currant extract, or an analog of any of the above.

The systems and methods described herein provide scleral translocation and elastomodulation (hereinafter “STEM”) adjustment treatment procedure to treat glaucoma, suitable for combination with any other treatment pattern or modality disclosed herein. The energy to shrink and/or plasticize the inner portion of the eye can include one or more of thermal energy, RF energy, mechanical energy, electrical energy, microwave energy, light energy, high intensity ultrasound energy, or ultrasound energy. The energy can shrink and/or plasticize the tissue by heating the tissue to a suitable temperature without substantially weakening the tissue, such as within a range from about 50° C. to 70° C. Heating the tissue can increase the elasticity of the tissue. In many embodiments, the heat is applied such that the outer portion of the tissue remains substantially viable so as to inhibit post-operative pain and swelling. While in many embodiments the energy can be applied through the conjunctiva and/or epithelium, the energy can also be applied with the conjunctiva and/or epithelium moved away from the sclera. The energy source can be the same energy source used to cross-link the eye, as described herein, or a different energy source.

FIG. 3 illustrates common fluid outflow paths of the eye including the location of the limbus and Schlemm's canal. Glaucoma may be caused by obstruction to one or more fluid outflow paths. Aqueous humor is produced by the ciliary body processes and secreted into the posterior chamber. From there it flows through the narrow cleft between the anterior surface of the lens and the posterior surface of the iris, into the anterior chamber. The fluid may exit the anterior chamber via the trabecular outflow route and/or the uveoscleral outflow route into the anterior chamber angle (drainage canal) and out of the eye. The fluid may alternatively or in combination exit the anterior chamber through the iris surface and capillaries. In the trabecular outflow route, the fluid exits the anterior chamber and travels out of the eye via the trabecular meshwork. The fluid then drains directly into Schlemm's canal, an endothelial cell-lined channel at the limbus (where the cornea and sclera meet), or indirectly through collector channels and then into the episcleral venous system. In the uveoscleral outflow route, aqueous humor seeps through, around, and between tissues, including the supraciliary space, ciliary muscle, suprachoroidal space, choroidal vessels, emissarial canals, sclera, and lymphatic vessels, but does not have a well-defined structural pathway like the trabecular route. Blockage of one or more outflow pathways may increase intraocular pressure (IOP) and cause glaucoma. Reduction of IOP may treat glaucoma. Common mechanisms by which these mechanisms are blocked include closing of the anterior chamber angle, blockage of pores and/or vacuoles in Schlemm's canal, blockage (and/or collapse) or Schlemm's canal, blockage of uveo-sclera outflow (for example blockage of vacuoles or pores of the perilimbic sclera), and any combination thereof.

Possible outcomes of the glaucoma treatment protocols described herein may include restoration of outflow through one or more of the three primary outflow pathways. Treatment may be used to open closed angle, dilate and/or stretch the trabecular meshwork, dilate and/or stretch Schlemm's canal, increase porosity and/or dilate vacuoles of the perilimbic sclera, or any combination thereof.

Treatment may be patterned or located so as to open closed angle, open Schlemm's canal and/or the trabecular meshwork, open collector channels, change fluid bypass characteristics, stretch the trabecular meshwork, and/or improve the uveo-sclera outflow pathway. For example, angle closure may be treated with one or more paralimbal annulus, for example two or more paralimbal annuli. Schlemm's canal closure and/or trabecular meshwork closure may be treated with one or more juxtacanalicular annuli, for example two or more juxtacanalicular annuli, for example a first annulus radially inward from Schlemm's canal and a second annulus radially outward from Schlemm's canal. Increased porosity of the perilimbal sclera and/or dilation or vacuoles may include treatment to relax or stretch the supra-ciliary and/or sub-conjunctival sclera alone or in combination with treatment at the pars plana and/or pars plicata. Treatment to increase porosity may provide reduced intraocular pressure as a stand-alone treatment or in combination with other treatment methods or patterns as described herein. Increased porosity in the mid-stromal near the pars plana and/or pars plicata may for example enhance hydraulic conductivity/transport of the supra-choroidal, ciliary, and/or lymphatic fluid outflow pathways. Treatment may be patterned to flatten the iris in order to open closed angle. Treatment above the base of the iris root or the roof of the ciliary body may dilate Schlemm's canal and/or stretch the trabecular meshwork. In some cases, it may be beneficial to treat more than one region in a single patient. For example, treatment may be patterned so as to open angle, open Schlemm's canal and/or the trabecular meshwork, and increase porosity and/or dilate vacuoles of the perilimbic sclera. Treatments directed towards multiple indications may take around 1 minute to about 3 minutes to complete. Treatments directed towards dilating Schlemm's canal may be used to anteriorly expand the roof of Schlemm's canal by about 30 um to about 100 um. Changes in the cross-section of the trabecular meshwork and/or Schlemm's canal may cause scleral pores to expand and increase outflow, thereby improving glaucoma.

Treatment using the systems and methods described herein may treat glaucoma improving homeostatic IOP mechanisms, so as to reduce pressure of the eye. For example, heating of one or more of the scleral, trabecular meshwork, or the ciliary body as described herein may induce one or more endogenous biological cellular cascades which may lead to improvements in outflow function. Without being bound by any particular theory, heating of the target tissue with energy such as laser energy may stimulate heat shock protein (HSP) activation, which may lead to normalized cell functions, normalized cytokine expression, and improved auto-regulation of IOP. Such improved function may, for example, be related to opening of one or more of the collector channels, ostia of the collector channels, or the trabecular meshwork.

Treatment using the systems and methods described herein may be configured so as to treat glaucoma without substantial corneal thinning as measured by axial pachymetry using OCT or US imaging. Corneal thinning may be less than about 15%, for example less than about 10%, or less than about 5% after treatment. Typical day to day variability of corneal epithelial thickness (axial pachymetry) as measured by OCT may be ±15 um 95% CI. Treatment using the systems and methods described herein may be configured to treat glaucoma with a change (increase or decrease) in corneal epithelial thickness of nor more than 50 um, for example no more than 25 um, or no more than 15 um.

Treatment using the systems and methods described herein may be used to generate or dilate vacuoles in the sclera. Low-powered ultrasound or infrared energy may be delivered to the tissue over a period of time in order to shrink the treatment locations and induce strain within the sclera which can cause stretching and translocation of other parts of the sclera which may in turn produce vacuoles. For example, treatment with 100 mW to 300 mW 2.1 um laser with a scan speed of 25 mm/sec, spot size of 200-300 um may be used to deposit about 4 to about 50 Joules of energy to the sclera in about 8 to about 100 seconds. 360 degree thermal treatment of the sclera may generate vacuoles and improve scleral outflow. Alternatively or in combination, scleral vacuoles may be created by targeting collagen in the sclera using a laser with a wavelength of about 6 um. Alternatively or in combination, scleral vacuoles may be created using a combination of low-powered light energy and electro-sprayed micro-droplets for non-thermal cavitation as described herein.

Table 3 shows possible treatment parameters which may be used with the system for the methods described herein. The methods and system described herein may be operated with any combination of the parameters listed in Table 3. Exemplary ranges are given for each treatment scheme with preferred values in parentheses next to the ranges. It will be understood by one of ordinary skill in the art that possible parameter values may be taken from within the ranges described, as well as by any two values therein.

Treatment to generate sclera vacuoles may include aggressive treatment using a 360° non-contiguous/non-overlapping annulus of treatment spots in order to generate micropockets which can act as vacuoles in the scleral tissue at and about the pars plana. Juxtacanalicular treatment of Schlemm's canal (“SC”) may include one or more 360° treatment annulus radially inward of Schlemm's canal (in the cornea) and one or more 360° treatment annulus radially outward of Schlemm's canal (in or near the sclera). In some cases, both the inner and outer treatment annuli may have the same or different treatment parameters. For example, both treatment annuli may have parameters configured to generate tissue shrinkage as shown in the third column and described herein. Alternatively or in combination, one or more inner treatment annulus may have parameters configured to elasticize (or relax) the tissue as shown in the fourth column. The treatment parameters shown for treatment of the cornea radially inward of Schlemm's canal may be less aggressive than the treatment parameters used for the sclera in order to provide additional protection to the cornea. In some instances, dilation of Schlemm's canal may occur without shrinking the tissue of the cornea inward of Schlemm's canal. In some instances, dilation of Schlemm's canal may occur using decrustation/elasticizing treatment of the cornea inward of Schlemm's canal.

TABLE 3
Exemplary parameters for various treatment locations and effects

| | | Radially outward | Radially inward treatment |
|---|---|---|---|
| Parameter | Sclera Vacuoles | treatment of SC | of SC |
| Wavelength | 1.5-2.1 μm | 1.5-2.1 μm | 1.5-2.1 μm |
| Total Treatment | 0.1-3 min (2 min) | 2-5 min (3 min) | 0.1-5 min (0.5 min) |
| Time | | | |
| Laser Power | 200 mW-1 W (400 mW) | 300-500 mW | 150-300 mW (200 mW) |
| | | (400 mW) | |
| Scan Speed | 10-50 mm/s | 10-50 mm/s | 10-50 mm/s (20 mm/s) |
| | (20 mm/s) | (20 mm/s) | |
| Tissue temperature | 80 C.-100 C. or more | 60 C.-70 C. | 50 C.-60 C. |
| Beam diameter | 0.4-0.8 mm (0.6 mm) | 0.2-0.6 mm (0.6 mm) | 0.2-0.5 mm (0.6 mm) |
| Treatment Location | Pars plana | 0.2-2 mm (0.5 mm) | 0.2-1 mm (0.5 mm) |
| | | radially outward from | radially inward from |
| | | distal wall of SC | proximal wall of SC |
| Treatment Pattern | 360° non-contiguous | 180°-360° annulus | 180°-360° annulus (or |
| | annulus of 10 μm-2 mm | (or patterned to mimic | patterned to mimic |
| | spots | SC/limbus) | SC/limbus) |
| Treatment Effect | Formation of vacuoles | Shrinkage | Decrustation |
| | (micropockets) | | (Elasticization/Relaxation) |

Patients may be treated using the system and methods described herein in order to avoid hitting (i.e. spare or protect) or reduce the amount of energy deposited at sensitive regions and structures of the eye. The use of a cooling structure, for example a chilled contact lens, the wavelength of the treatment, the treatment dosage, the treatment patterning (for example using a “mark and jump” technique as described herein), and other features of the system and methods described herein may be configured to protect structures such as the epithelium, conjunctiva, Bowman's membrane, epi-scleral veins, Schlemm's canal, trabecular meshwork, limbal stem cells, ciliary body, nerves, collector channels, and/or vasculature of the eye.

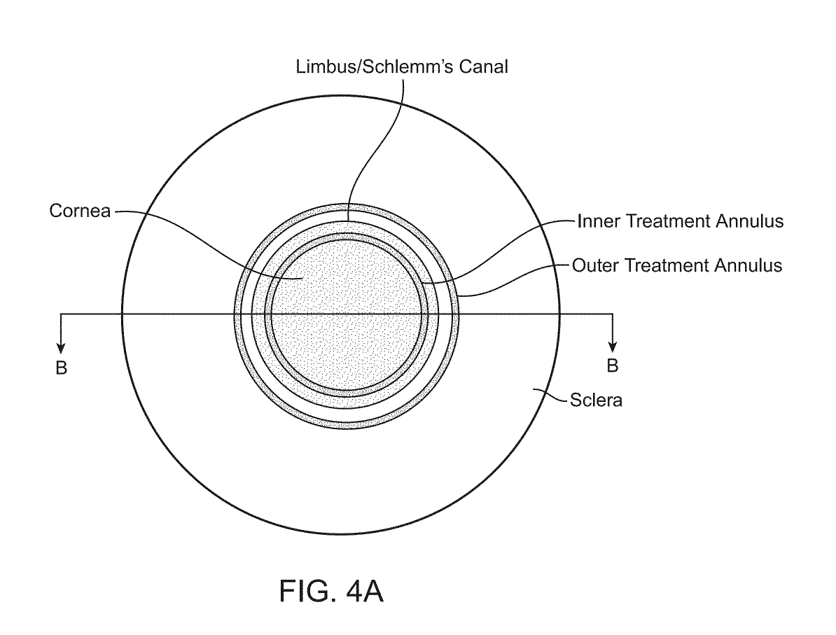

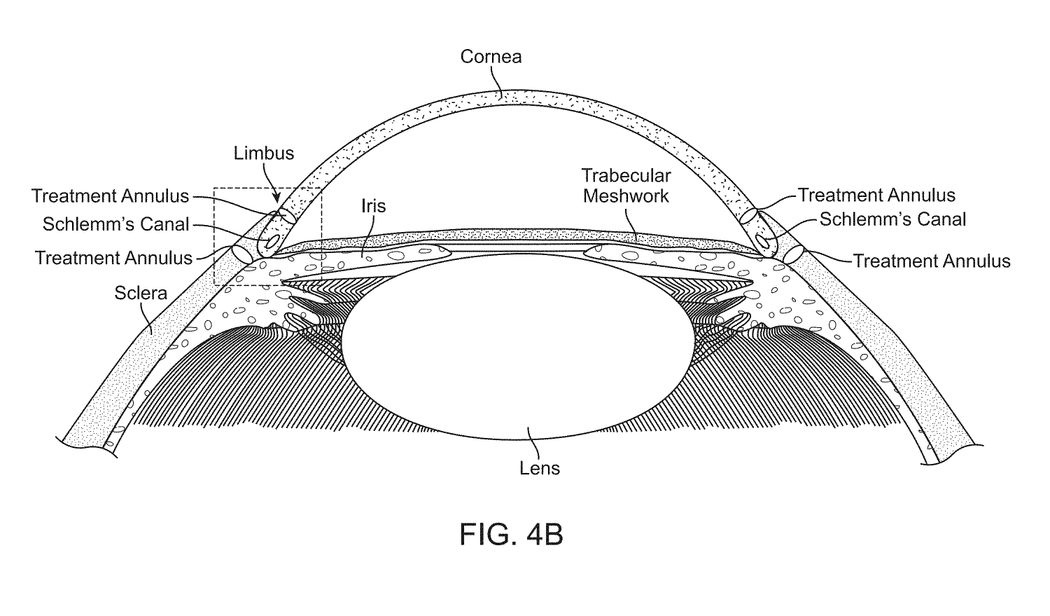

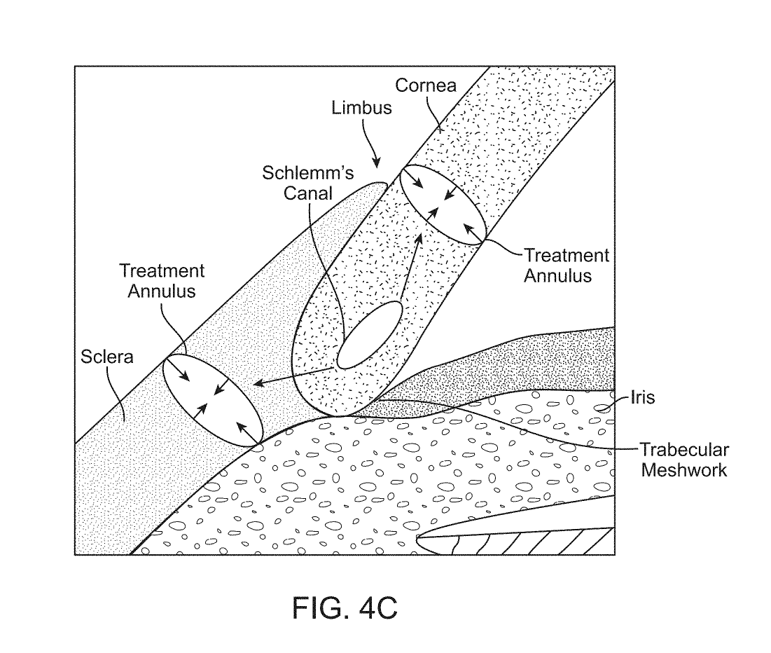

FIG. 4A illustrates a possible treatment pattern which may be used to dilate Schlemm's canal. Two or more annuli or rings may be patterned to straddle Schlemm's canal. For example, an inner treatment annulus may be positioned radially inward of Schlemm's canal and an outer treatment annulus may be positioned radially outward of Schlemm's canal. Juxtacanalicular treatment of Schlemm's canal with the inner annulus separated from the outer annulus the may induce improved stretching between the annuli similar to a suspension effect and may improve dilation of Schlemm's canal and/or porosity of the sclera. The inner treatment annulus may be located within the cornea. The outer treatment annulus may be located within the sclera.

The inner annulus may comprise a single continuous treatment annulus. Alternatively, the inner annulus may comprise a single discontinuous treatment annulus with a predetermined amount of space between “dashes” or “spots” of treatment along inner annulus. In some instances, the inner annulus may comprise a plurality of treatment annuli. The inner annulus may have a pre-determined width which is made up by the plurality of treatment annuli. The plurality of annuli may be overlapping or non-overlapping. For example, the plurality of annuli may be overlapping so as to generate the inner annulus with a width greater than the spot size of the energy beam (e.g. laser beam or HIFU beam). The plurality of annuli may be non-overlapping, with a pre-determined radial distance between each of the plurality of annuli making up the inner annulus.

The outer annulus may comprise a single continuous treatment annulus. Alternatively, the outer annulus may comprise a single discontinuous treatment annulus with a predetermined amount of space between “dashes” or “spots” of treatment along outer annulus. In some instances, the outer annulus may comprise a plurality of treatment annuli. The outer annulus may have a pre-determined width which is made up by the plurality of treatment annuli. The plurality of annuli may be overlapping or non-overlapping. For example, the plurality of annuli may be overlapping so as to generate the outer annulus with a width greater than the spot size of the energy beam (e.g. laser beam or HIFU beam). The plurality of annuli may be non-overlapping, with a pre-determined radial distance between each of the plurality of annuli making up the outer annulus.

The inner and the outer annulus may be separated by a distance of about 0.5 mm to about 4 mm. The inner treatment annulus may be located within about 2 mm radially inward of Schlemm's canal. The inner treatment annulus may for example be located about 1 mm radially inward of the innermost edge of Schlemm's canal. The outer treatment annulus may be located within about 2 mm radially outward of Schlemm's canal. The outer treatment annulus may for example be located about 1 mmm radially outward of the outermost edge of Schlemm's canal.

The annuli may be circular as shown. Alternatively, the annuli may be oval, elliptical, egg-like, non-circular, non-elliptical, asymmetrical, or patterned to mimic (e.g. correspond to) the shape of Schlemm's canal or the limbus which may have a non-regular shape. The annuli may be shaped roughly the same as one another or may be differently shaped.

FIGS. 4B and 4C show a cross-section of the eye of FIG. 4A taken along line B-B with treatment annuli located on either side of Schlemm's canal. FIG. 4C shows a magnified view of the dashed box indicated in FIG. 4B. The two treatment annuli are positioned to straddle Schlemm's canal. Shrinkage of the tissue within the treatment annuli (as indicated by the arrows pointing inward in the treatment locations on FIG. 4C) may generate sufficient strain within the scleral tissue surrounding the treatment locations to open Schlemm's canal. The tissue surrounding Schlemm's canal may be urged towards the treatment locations following treatment (as indicated by the arrows extending from Schlemm's canal in FIG. 4C) which may in turn “pull” on the roof of Schlemm's canal and cause dilation and/or translocation of Schlemm's canal, thereby improving aqueous outflow for treatment of glaucoma.

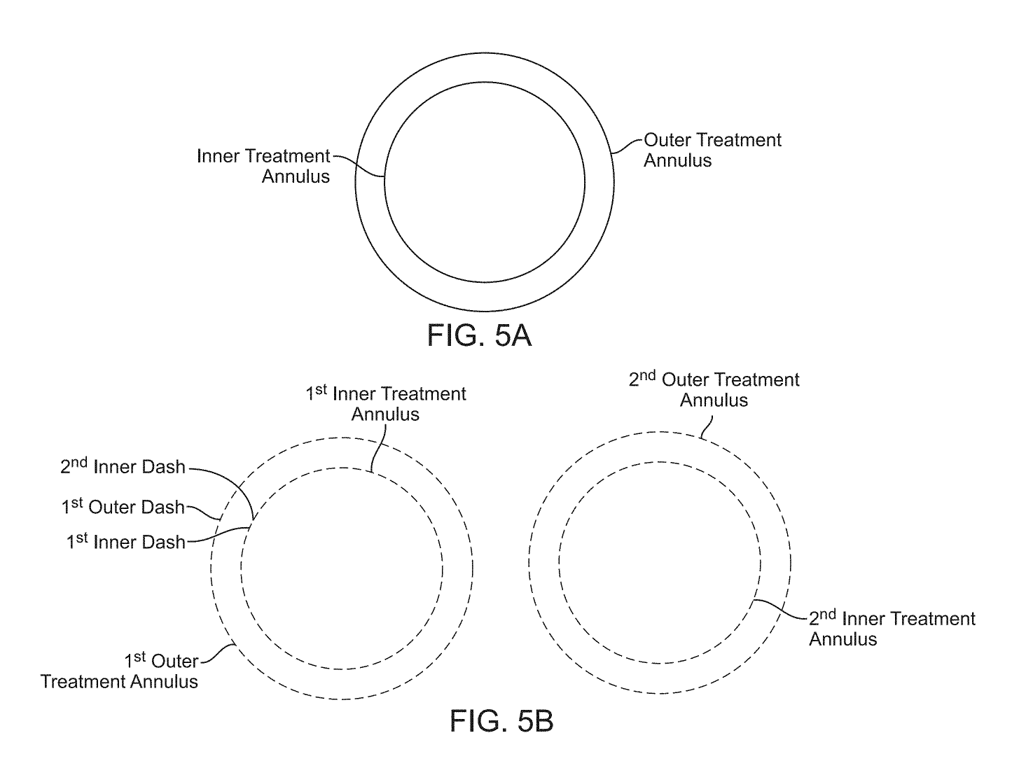

FIGS. 5A-5B show possible juxtacanalicular treatment patterning which may be used to treat glaucoma and dilate Schlemm's canal. FIG. 5A shows two circular annuli, an outer annulus and an inner annulus, which may be patterned onto the eye to straddle Schlemm's canal as described herein. While circular annuli are shown, it will be understood that any of the treatment patterns described herein may be used as desired. In some instances, the inner annulus may be created by scanning the energy onto the eye in a continuous annulus. Alternatively or in combination, the inner annulus may be created by scanning the energy onto the eye using overlapping pulses from a pulsed laser with a high pulse rate such that the pulses overlap. Alternatively or in combination, the inner annulus may be created by scanning the energy onto the eye using non-overlapping pulses. For example, a “mark and jump” technique may be used to generate “dashes” or “marks” of treatment locations along the inner annulus separated by untreated tissue along the inner annulus (see FIG. 5B for an example of a dashed pattern). In a second repetition, the untreated tissue may be treated with “dashes” of energy while the previously treated locations may be “jumped” over and left untreated so as to interleave the two repetitions. When both repetitions have been completed, the inner annulus may be substantially similar to an annulus generated by continuous deposition of energy in a single repetition along the path shown in FIG. 5A. In some instances, the outer annulus may be created by scanning the energy onto the eye in a continuous annulus. Alternatively or in combination, the outer annulus may be created by scanning the energy onto the eye using overlapping pulses from a pulsed laser with a high pulse rate such that the pulses overlap. Alternatively or in combination, the outer annulus may be created by scanning the energy onto the eye using non-overlapping pulses. For example, a “mark and jump” technique may be used to generate “dashes” or “marks” of treatment locations along the outer annulus separated by untreated tissue along the outer annulus. In a second repetition, the untreated tissue may be treated with “dashes” of energy while the previously treated locations may be “jumped” over and left untreated so as to interleave the two repetitions. When both repetitions have been completed, the outer annulus may be substantially similar to an annulus generated by continuous deposition of energy in a single repetition along the path shown in FIG. 5A.

In some instances, the inner and outer annuli may be created using a modified “mark and jump” interleaving technique as shown in FIG. 5B. The scanner may be configured to “jump” between a first “dash” on a first inner treatment annulus to a first “dash” on a first outer treatment annulus, then back to the first inner treatment annulus to generate a second “dash” at a predetermined distance from the first “dash” on the first inner treatment annulus. This zig-zag-like pattern of marking between the first inner annulus and the first outer annulus may continue for 360 degrees. The pattern may then be shifted so as to create a second inner treatment annulus and a second outer treatment annulus on a second 360 degree pass using the zig-zag mark and jump patterning. The “dashes” of the second inner treatment annulus may be patterned so as to interleave with and/or overlap with the “dashes” of the first inner treatment annulus. The “dashes” of the second outer treatment annulus may be patterned so as to interleave with and/or overlap with the “dashes” of the first outer treatment annulus. The final energy deposition pattern may be substantially continuous similar to the pattern shown in FIG. 5A.

In some instances, the “mark and jump” technique may be used to generate “dashes” or “marks” of treatment locations along the inner annulus and outer annulus such that a single continuous treatment annulus is generated in a single 360 degree interleaving rotation of the energy source. For example, the scanner may be configured to “jump” between a first “dash” on the first inner treatment annulus to a first “dash” on a first outer treatment annulus, then back to the first inner treatment annulus to generate a second “dash” which begins at the edge of the first “dash” on the first inner treatment annulus. This zig-zag-like pattern of marking between the first inner annulus and the first outer annulus may continue for 360 degrees such that the final energy deposition pattern after one 360 degree repetition may be substantially continuous similar to the pattern shown in FIG. 5A.

As described above, energy (e.g. pulses of energy) may be directed into an eye with an annular pattern, e.g. using a delivery system. The energy may be used to shrink and/or relax a sclera of the eye. The annular pattern of energy may in some instances be a shrinkage energy. In some instances, the annular pattern of energy may be a relaxation energy.

The annular pattern may comprise an inner dimension (e.g. inner treatment annulus shown in FIG. 5A) and an outer dimension (e.g. outer treatment annulus shown in FIG. 5A). The inner dimension may be located radially inward from a Schlemm's canal. The outer dimension may be located radially outward from the Schlemm's canal. The energy may be may be utilized in treating glaucoma of the eye and/or a refractive error of the eye. In some instances, the annular pattern may include a first pattern and/or a second pattern. The first pattern may treat a glaucoma of the eye while the second pattern may treat a refractive error of the eye. The first pattern may open Schlemm's canal while the second pattern may dilate scleral vacuoles.

In some instances, the energy may be directed to a plurality of treatment locations. The plurality of treatment locations may be located on either side of the plurality of Schlemm's canal locations. The plurality of treatment locations may comprise a first plurality of treatment locations. The first plurality of treatment locations may be positioned radially inwardly from the plurality of Schlemm's canal locations toward an optical axis of the eye. Optionally, the plurality of treatment locations may comprise a second plurality of treatment locations. The second plurality of treatment locations may be located radially outwardly from the plurality of Schlemm's canal locations. Optionally, the first and/or second plurality of treatment locations may each comprise or form an annular pattern described above. For example, the first plurality of treatment locations may refer to the dashes of the 1^{st} inner treatment annulus shown in FIG. 5B (e.g. including 1^{st} inner dash and 2^{nd} inner dash). As another example, the second plurality of treatment locations may refer to the dashes of the 1^{st} outer treatment annulus shown in FIG. 5B (e.g. including 1^{st} outer dash). In some instances, the first plurality of treatment locations may be within a range from about 0.5 mm to about 4 mm of the second plurality of treatment locations. Optionally, the first plurality of treatment locations may be distanced equal to or less than about 0.1 mm, 0.2 mm, 0.3 mm, 0.4 mm, 0.5 mm, 0.75 mm, 1 mm, 1.5 mm, 2 mm, 2.5 mm, 3 mm, 3.5 mm, 4 mm, 4.5 mm, 5 mm, 5.5 mm, 6 mm, 6.5 mm, 7 mm, or 8 mm away from the second plurality of treatment locations. Optionally, energy pulses directed at the first plurality of treatment locations may not overlap with the second plurality of treatment locations. Alternatively, energy directed at the first plurality of treatment locations may overlap with each other to define a first arc and the second plurality of pulses may overlap with each other to define a second arc with a gap between the first arc and the second arc.

In some instances, the energy discussed herein may be directed to the first plurality of treatment locations and the second plurality of treatment locations in alternating turns or sequences. This alternating treatment may decrease a movement of tissue at a location between the first plurality of locations and the second plurality of treatment locations. Alternatively or in addition, this alternating treatment may inhibit biasing of tissue at the first plurality of treatment locations or the second plurality of treatment locations. Alternating the treatment between the first plurality of treatment locations and the second plurality treatment locations may comprise treating a first portion of the first plurality of treatment locations (e.g. 1^{st} inner dash shown in FIG. 5B) followed by a first portion of the second plurality of treatment locations (e.g. 1^{st} outer dash shown in FIG. 5B) followed by a second portion of the first plurality of treatment locations (e.g. 2^{nd} inner dash shown in FIG. 5B). Optionally, the locations of the second portion of the first plurality of treatment locations may be interspersed between locations of the first portion of the first plurality of treatment locations.

In some instances, locations of the second portion of the first plurality of treatment locations are not interspersed between locations of the first portion of the first plurality of treatment locations, the first portion defining a first perimeter, the second portion defining a second perimeter and optionally wherein the first portion defines a first annular pattern and a second annular pattern radially separated from each other with reference to an optical axis of the eye. For example, both the 1st inner treatment annulus and the 1^{st} outer treatment annulus shown in FIG. 5B may be of first plurality of treatment locations, and the first and second portions of the first plurality of treatment locations may be located on different inner and outer treatment annuli.

In some instances, the plurality of treatment locations described herein may comprise a treatment pattern shown, or traceable on a two-dimensional anterior image of the eye. In some instances, the 2-D treatment pattern may be projected onto an eye. In some instances, the 2-D treatment pattern may be displayed on an anterior image of the eye. Optionally, the 2D treatment pattern projected onto the eye may comprise a plurality of locations on either side of a Schlemm's canal on anterior layer of the eye, the anterior layer selected from the group consisting of a cornea of the eye and a sclera of the eye.

In some instances, the plurality of treatment locations described herein may comprise a treatment pattern shown, or traceable on a three-dimensional anterior image of the eye. In some instances, a 2-D treatment pattern may be projected onto an eye. In some instances, the 2-D treatment pattern may be displayed on an anterior image of the eye. Optionally, the 2D treatment pattern projected onto the eye may comprise a plurality of locations on either side of a Schlemm's canal on anterior layer of the eye, the anterior layer selected from the group consisting of a cornea of the eye and a sclera of the eye. In some instances, a 3-D treatment pattern may be projected onto an eye. In some instances, the 3-D treatment pattern may be displayed on an anterior image of the eye. Optionally, the 3-D treatment pattern projected onto the eye may comprise a plurality of locations on either side of a Schlemm's canal on anterior layer of the eye, the anterior layer selected from the group consisting of a cornea of the eye and a sclera of the eye.

The energy described above may originate from an energy source, e.g. a laser, pulsed laser, ultrasound energy source, a continuous laser, a pulsed ultrasound transducer, a HIFU array, or a phased HIFU array. In some instances, the energy source may be configured to generate energy having a wavelength within a range from about 1.5 um to about 2.1 um. Optionally, the energy may have a wavelength equal to or less than about 0.8 um, 1 um, 1.2 um, 1.4 um, 1.6 um, 1.8 um, 2.0 um, 2.2 um, 2.4 um, 2.6 um, 2.8 um, or 3.0 um.

In some instances, the energy source may direct energy to the eye with a power from about 50 mW to about 900 mW. Optionally, the energy source may direct energy to the eye with a power from about 100 mW to about 700 mW. Optionally, the energy source may direct energy to the eye with a power from about 200 mW to about 400 mW. Optionally, the energy source may direct energy to the eye with a power equal to or less than about 10 mW, 25 mW, 50 mW, 150 mW, 200 mW, 300 mW, 400 mW, 500 mW, 600 mW, 700 mW, 800 mW, 900 mW, 1 W, 1.2 W, 1.4 W, 1.6 W, 1.8 W, or 2.0 W.

In some instances, the energy directed to the eye may have a total energy within a range from about 4 J to about 90 J. Optionally, the energy directed to the eye may have a total energy within a range from about 5 J to about 50 J. Optionally, the energy directed to the eye may have a total energy equal to or less than about 1 J, 2 J, 5 J, 10 J, 15 J, 20 J, 25 J, 30 J, 35 J, 40 J, 45 J, 50 J, 60 J, 70 J, 80 J, 90 J, 100 J, 120 J, or 150 J.

In some instances, the energy may be directed to the eye for a duration (e.g. treatment time) within a range from about 4 to 200 seconds. Optionally, the energy may be directed to the eye for a duration (e.g. treatment time) within a range from about 8 to 100 seconds. Optionally, the energy may be directed to the eye for a duration equal to or less than about 2, 4, 6, 8, 10, 12, 15, 20, 25, 30, 35, 40, 45, 50, 60, 70, 80, 90, 100, 120, 140, 160, 180, 200, 220, 240, 260, 280, or 300 seconds.

In some instances, the energy directed to the eye may comprise a cross-sectional beam spot size within a range from about 100 to about 500 um. Optionally, the spot size may be within a range from about 150 to about 400 um. Optionally, the spot size may be within a range from about 200 to about 300 um. In some instances, the spot size may be a spot size that is applied to tissue near the Schlemm's canal. In some instances, the cross-sectional beam spot size may be within a range from about 100 to about 800 um, for example about 600 um.

In some instances, the energy may be scanned along the eye with a scan rate within a range from about 10 to about 100 mm/second. Optionally, the energy may be scanned along the eye within a range from about 12 to about 50 mm per second. Optionally, the energy may be scanned along the eye within a range from about 20 to about 30 mm/second. In some instances, the energy may be scanned along the eye at a rate equal to or less than about 5 mm/s, 10 mm/s, 15 mm/s, 20 mm/s, 25 mm/s, 30 mm/s, 35 mm/s, 40 mm/s, 45 mm/s, 50 mm/s, 55 mm/s, 60 mm/s, 70 mm/s, 80 mm/s, 90 mm/s, or 100 mm/s.

The energy delivered to tissue within a treatment region can be fractionated by exposing tissue to energy at separate times. For example, energy can be delivered to a specific location within the treatment region at a first time, and delivered again at a second time so as to fractionally deliver the energy to location of the tissue. The number of times energy can be repeatedly delivered to a region of tissue can be greater than two, and can comprise for example 3 to 10 repeated exposures to a region of tissue. The amount of time between successive energy exposures to the region of tissue can be related to a thermal relaxation time of the tissue and generally comprises an amount of time greater than a thermal relaxation time. The thermal relaxation time generally corresponds to an amount of time that it takes for a region of tissue to decay from a peak temperature, e.g. about 60 degrees C., to a temperature within about 37% (1/e) of the pre-exposure temperature, e.g. about 5 degrees C. The thermal relaxation time can be determined experimentally with a thermal camera or computer modelling, for example, as is known to one of ordinary skill in the art. The thermal relaxation time can be related to tissue thermal conductivity and several treatment parameters such as the size of the region exposed to energy, e.g. beam diameter, and the amount of time a region of tissue is exposed to energy with each scan, e.g. scan rate.

The amount of time between repeated applications of energy to a region of tissue can be related to the scan rate and size of the energy beam at the location. For example, with a scan at a diameter of 12 mm, the total length scanned with a first 360 degree pass comprises about 37.7 mm (12 mm*3.14). With a scan rate of 10 mm/s, the total amount of time for a complete 360 degree scan comprises about 3.7 s. For a beam diameter of about 0.600 mm, and a scan rate of 10 mm/s, the amount of time which a region of tissue along the scan path is exposed comprises about 0.06 s (60 ms). For repeated scans of the treatment beam along a similar trajectory with substantially the same diameter without pauses between scans, there is a delay of about 3.7 s between repeated exposures at the tissue location for continuous scanning. For repeated scans of the beam to the tissue location, the fractionation of the energy delivered to the tissue location corresponds to a duty cycle of about 0.159% at the tissue location. Within this framework, a person of ordinary skill in the art can configure the systems as described herein to fractionate the energy delivered to a tissue location in many ways, for example with scanning pulsed energy sources, repeatedly applying substantially fixed energy sources (e.g. an annular handpiece), or scanning continuous energy sources, and combinations thereof.

In some instances, a plurality of repetitions with substantially the same treatment pattern may be used to increase the total amount of energy deposited at the treatment location(s). Increased total energy deposition may lead to a more complete, and optionally more stable, transformation within the tissue. By using a plurality of repetitions to fractionate the energy, the total amount of energy deposited at the treatment location may be fractionated, with the amount of energy deposited per repetition reduced compared to the total energy deposited at the location with the treatment, which may provide for more uniform treatment. Repetitions may be overlapping, for example partially or fully overlapping, for example, and may be offset from each other by a distance, such as a predetermined distance. The number of repetitions may for example be within a range of about 1 to about 1000 repetitions, for example about 10, 20, 30, 40, 50, 100, 200, 300, 400, 500, or 1000 repetitions, or within a range bounded by any two of the preceding values.

In some instances, a plurality of repetitions of the laser may be used to fractionate the treatment energy delivered to a treatment location. Fractionation of the treatment may be provided to increase the total energy deposition at a desired treatment location, or within a desired treatment pattern, while decreasing tissue damage by spacing the energy deposition out over time. For example, a desired treatment location or pattern may receive a plurality of energy pulses over a pre-determined amount of time, with a pre-determined delay between each of the plurality of energy pulses, in order to deposit the desired total energy to the tissue and induce the desired changes (e.g. stretching, relaxation, shrinking, etc.) without overheating or over-dosing the tissue. The fractionation of energy delivery can provide more uniform tissue effects, such as more uniform shrinkage or allow for increasing an amount of tissue effect, such as stretching, by allowing for repeated exposures.

Fractionation of the treatment energy delivered to a treatment location may for example allow for delivery of energy to the tissue without inducing cell death at the treatment location. Fractionation of the treatment energy may for example allow for delivery of energy to the tissue at a level below which the cells are killed but above which certain biological stress mechanisms are induced. For example, fractionation of the treatment energy may be used to deliver energy to the tissue in order to induce a cellular response which may improve IOP, such as the induction of HSP and/or cytokine expression as described herein which may be beneficial in regulating IOP.

Any of the systems described herein may comprise a processor configured with instructions to fractionate delivery of the energy to each of a plurality of treatment locations with repeated delivery of energy to said each of the plurality of treatment locations. The fractionation optionally may comprise an amount of fractionation within a range from about 0.1% to about 10%, optionally wherein the range is from about 0.2% to about 5%. The fractionation may optionally correspond to an amount of exposure time for said each location and a time delay between successive exposures to said plurality of locations.

The processor may be configured with instructions to configured repeatedly deliver the energy to each the plurality of treatment locations with a time delay in order to fractionate delivery of energy to said each of the plurality of treatment locations. The time delay may be within a range from about 10 millisecond (ms) to about 60 seconds, for example within a range from about 100 ms to about 30 seconds, within a range from about 500 ms to about 15 seconds, or within a range from about 1 second (s) to about 10 seconds.

While a laser is described for scleral translocation elasto-modulation (“STEM”), a person of ordinary skill in the art will be able to modify the treatment parameters such that any energy delivery apparatus as described herein may be used to acquire the desired results of treating glaucoma and reduction of intraocular pressure (IOP), for example ultrasound or HIFU.

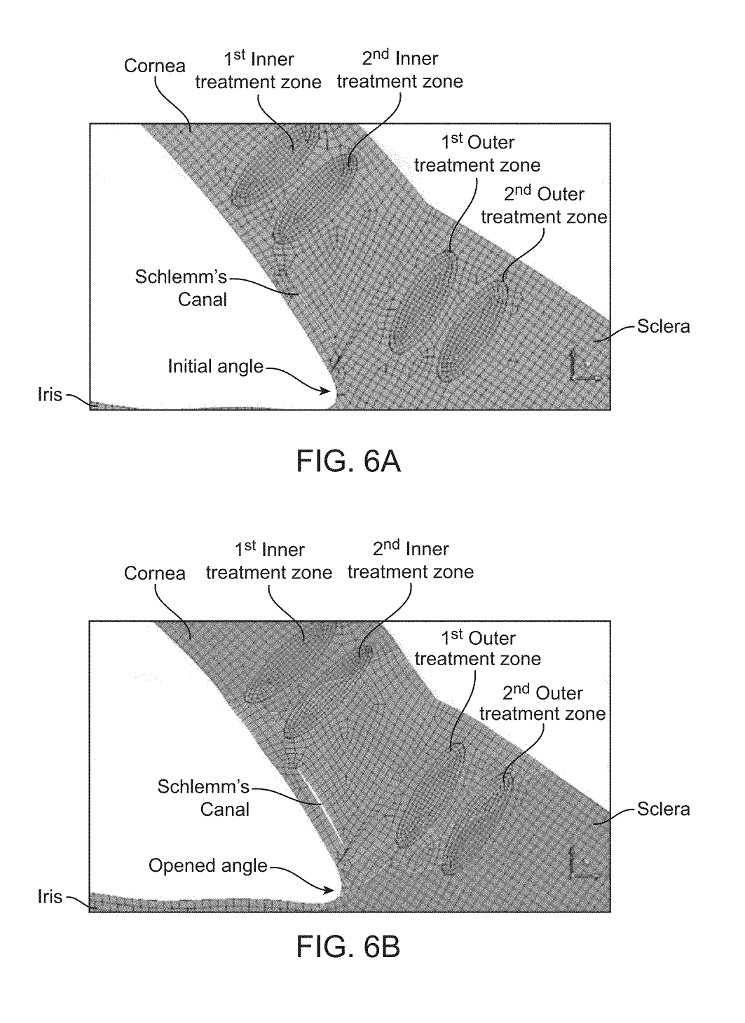

FIGS. 6A-6B show the results of a finite element analysis simulation of juxtacanalicular shrinkage treatment of Schlemm's canal in a simulated axis-symmetric eye with an IOP of 15 mmHg. The ANSYS 17.1 FEM simulation package was used to run the simulation. The cornea, sclera, ciliary body and iris, and laser spots were modeled as varying mesh distributions to mimic the biochemical properties of the tissue within those tissues. The cornea was modeled as hyperelastic, non-linear, and incompressible (with μ=1.2 MPa and D1—0 MPa⁻¹). The sclera was modeled as hyperelastic, non-linear, and incompressible (with μ=2.4 MPa and D1—0 MPa⁻¹). The ciliary body/iris was modeled as hyperelastic, non-linear, and incompressible (with μ=1.2 MPa and D1—0 MPa⁻¹). The laser treatment spots were modeled as hyperelastic, non-linear, and compressible (with μ=1.2 MPa and D1—0.001 MPa⁻¹). The cornea limbus was modeled as hyperelastic, non-linear, and compressible (with μ=3.8 MPa and D1—0 MPa⁻¹). The cornea under Schlemm's canal was modeled as hyperelastic, non-linear, and compressible (with μ=24 MPa and D1—0 MPa⁻¹). The outer edges of the sclera were fixed.

The effects of treating the eye with two annuli radially inward of Schlemm's canal and two annuli radially outward of Schlemm's canal were modeled. Non-overlapping annuli are shown but it will be understood by one of ordinary skill in the art that similar results may be obtained with overlapping annuli (generating a single inner annulus and a single outer annulus of treatment after energy deposition has been completed as described herein, for example as shown in FIGS. 4A and 5A). The first outer treatment zone and second inner treatment zone were positioned 500 um posterior and anterior to Schlemm's canal, respectively. The second outer treatment zone was positioned 200 um radially outward from the first outer treatment zone. The first inner treatment zone was positioned 200 um radially inward of the second inner treatment zone. The effects of the laser were simulated by volume reduction (i.e. shrinking) within the tissue at the indicated elliptical treatment locations. Schlemm's canal was modeled as an approximately 350 um wide closed channel prior to treatment.

FIG. 6A shows a simulated portion of the eye prior to treatment with an initial angle and a thin Schlemm's canal. FIG. 6B shows the simulation portion of the eye after treatment with Schlemm's canal expanded and angle opened. Localized effects were observed due to tissue shrinkage in the treatment zones. The spots shrunk by about 20% with respect their initial volume (as shown in FIG. 6A). Schlemm's canal opened to a width of about 16 um, more than five times its initial width of about 3 um. The initial length of the canal was elongated by about 10 um. The second inner treatment zone and the first outer treatment zone had a larger impact on the opening of Schlemm's canal than the more removed first inner treatment zone and second outer treatment zones. The first inner treatment zone and the second outer treatment zone contributed more to the about 3° opening of the chamber angle. The inventors believe that, by adjusting the treatment parameters, the juxtacanalicular treatments within about 1.5 mm of the Schlemm's canal of the eye can open the angle by an amount within a range from about 1 to 6 degrees. These results also suggest that the size, shape, and potion of the energy spots such as laser spots may be sensitive parameters for the opening of Schlemm's canal. The elliptical shape of the treated zones yielded tissue stretching and contraction tangential to the corneal surface. The simulation showed opening of Schlemm's canal as a result of the elliptical treatments. These results suggest that tissue shrinkage can used to expand Schlemm's canal as described herein.

The amount of tissue shrinkage and corresponding opening of the angle can be related to the treatment parameters as described herein, and repeated exposures with energy fractionation can be provided in order to increase or decrease the amount of angle opening. The processor can configured with instructions to fractionate delivery of the energy to each of the plurality of treatment locations with repeated delivery of energy to said each of the plurality of treatment locations in order to open the angel to a targeted amount within a range from about 1 degree to about 6 degrees. For example, additional energy and fractionation as described herein can be provided to the eye to increase the amount of opening of the angle. The fractionation may comprise an amount of fractionation within a range from about 0.1% to about 10%, and may be from about 0.2% to about 5%. The fractionation may correspond to an amount of exposure time for said each location and a time delay between successive exposures to said plurality of locations, so as to provide controlled amounts of tissue shrinkage without excessive heating. The fractionation may correspond to a plurality of exposure times to a plurality of locations and a plurality of delay times between a plurality of successive exposures. Additional or fewer locations may also be treated with energy fractionation as described herein.

These juxtacanalicular treatment locations were modeled such that the inner-most treatment zone and the outer-most treatment zone are generally within about 2 mm of the limbus, for example within about 1 mm of the limbus. These juxtacanalicular treatment locations can provide opening of the angle of the eye with a direct coupling between the sclera and the iris, for example without tensioning zonules of the eye. In the specific example shown in FIG. 6A, the inner-most treatment zone was modeled to be 700 um from Schlemm's canal (500 um plus 200 um for the first inner treatment zone), and the outermost treatment zone was modeled to be 700 um from Schlemm's canal (500 um plus 200 um for the second outer treatment zone).

Without being bound by any particular theory, the inventors believe that the opening of the angle by about 3 degrees is related to shrinkage of scleral tissue with relatively little opening being related to shrinkage at the corneal treatment locations. The shrinkage of the scleral tissue can induce movement of tissue coupled to the iris so as to induce tilting of the iris in order to open the angle. The iris can be mechanically coupled to other tissues of the eye, such as the ciliary body of the eye, in order to induce tilting of the iris so as to open the angle. Because of the penetration depths and locations shown in FIGS. 6A and 6B, the angle of the eye can be opened without changing tensioning on the zonules of the eye, that may otherwise induce a change in refraction of the eye as described in PCT application PCT/US2017/023092, filed on 17 Mar. 2017, entitled “EFFECTIVE OCULAR LENS POSITIONING METHODS AND APPARATUS”, although combination treatments can be used in accordance with the present disclosure. The scleral treatment locations within about 2.0 mm of Schlemm's can be used to open the angle without substantially changing properties of the lens of the eye, for example. The scleral treatment locations can be configured such that a majority of the treatment energy is located within about 1.5 mm of Schlemm's canal of the eye. The treatment locations can be configured in many ways, for example with instructions to the processor as described herein, with a hand piece having appropriately dimensioned light transmitting structures, or with geometric optics such as masks imaged onto the eye.

In some embodiments, the juxtacanalicular treatments correspond to centroid of the treatment positioned radially with respect to Schlemm's canal. The centroid of treatment can be determined in radial coordinates using known center of mass calculations as is known to one of ordinary skill in the art. The centroid of the treatment can be determined with respect to radial treatment locations. With reference to the 1^{st} inner treatment zone (700 um from Schlemm's canal) and the 2^{nd} inner treatment zone (500 um from Schlemm's canal), the centroid of the treatment will radially be located approximately 600 um from Schlemm's canal. With reference to the 1^{st} outer treatment zone (500 um from Schlemm's canal) and the 2^{nd} outer treatment zone (700 um from Schlemm's canal), the centroid of the treatment will be radially located approximately 600 um from Schlemm's canal, which the optical axis of the eye can be used to approximately define the center of the radial coordinate system.

While the juxtacanalicular treatments can be configured in many ways, in some embodiments treatment pattern is configured such that the majority of treatment energy is located within about 2 mm of the Schlemm's canal of the eye, for example within about 1.5 mm of the Schlemm's canal of the eye, in order to provide stretching of the trabecular meshwork and opening of Schlemm's canal of the eye. The associated increase in the angle of the eye can be within a range from about 1 degree to about 3 degrees, depending on the juxtacanalicular treatment locations, energy delivery times, and depth profile of treatment. A person of ordinary skill in the art can vary the juxtacanalicular treatment profiles as described herein in order to provide desired amounts to stretching to Schlemm's canal and the trabecular meshwork in order to increase outflow and increase the angle of the eye.

In embodiments using light energy, the wavelength and exposure time can be configured to provide shrinkage with a desired depth profile, and the cooling structure may be used to inhibit epithelial damage and to provide shrinkage zones beneath the epithelium. Also, depending on the amount of energy, cooling and treatment times used, a portion of the sclera through which energy passes (e.g. light energy) may comprise may be located above (i.e. anterior) to the treatment zone.

Treatment may be patterned to open Schlemm's canal and avoid translocation of Schlemm's canal. Alternatively, treatment may be patterned to open Schlemm's canal and cause translocation of Schlemm's canal. The order of patterning of the annuli on the eye may be important to generate a desired response. For example, translocation of Schlemm's canal may be biased towards the optical axis or center of the eye, and/or anteriorly, by delivering energy to the first and second inner treatment zones before the first and second outer treatment zones. Alternatively, translocation of Schlemm's canal may be biased away from the optical axis or center of the eye, and/or posteriorly, by delivering energy to the first and second outer treatment zones before the first and second inner treatment zones. Translocation of Schlemm's canal may alternatively be avoided by alternating between inner and outer treatment annuli so as to avoid biasing the response to either side. For example, treatment may being with the first inner treatment zone, then move to the first outer treatment zone, then move to the second inner treatment zone, and end on the second outer treatment zone.

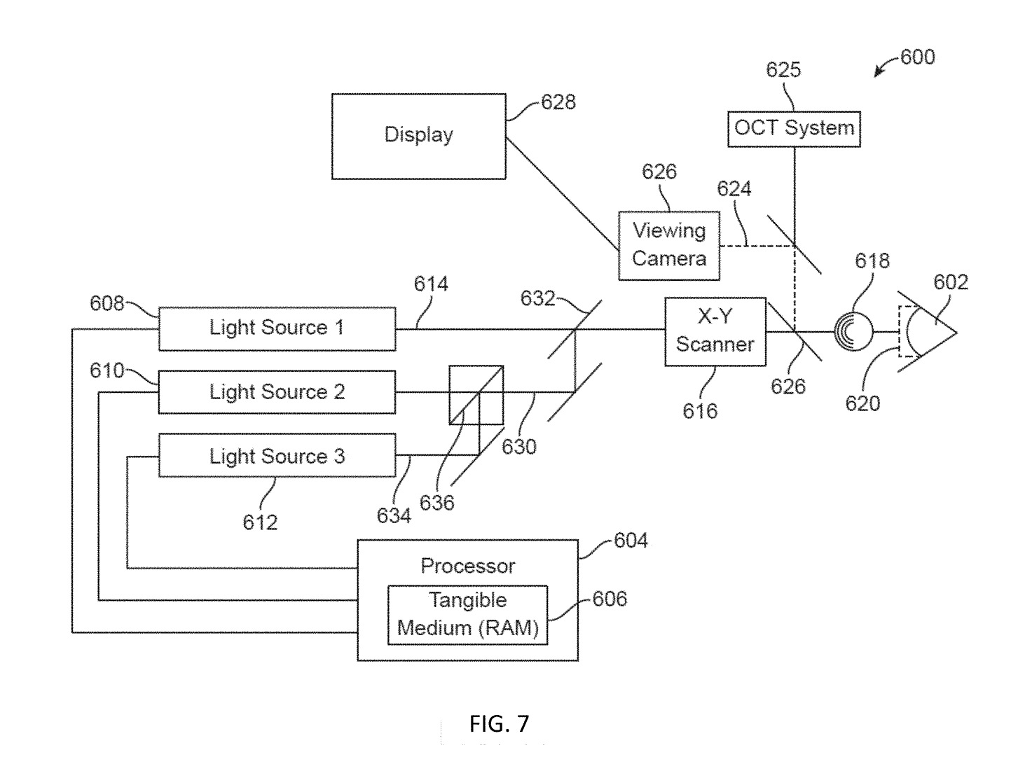

FIG. 7 illustrates a system 600 for treating an eye 602, in accordance with embodiments. The system 600 includes a processor 604 having a tangible medium 606 (e.g., a RAM). The processor 604 is operatively coupled to a first light source 608, an optional second light source 610, and an optional third light source 612. The first light source 608 emits a first beam of light 614 that is scanned by X-Y scanner 616 through an optional mask 618 and optional heat sink 620 onto the eye 602. The mirror 622 directs light energy from the eye 602 to a viewing camera 627 coupled to a display 628. An independent non-treatment light source for the optional viewing camera can be provided, for example. The mirror 622 may direct a portion of the light beam returning from eye 602 to the camera 627, for example. The second light source 610 emits a second beam of light 630 that is combined by a first beam combiner 632 with the first beam of light 614 prior to passing through X-Y scanner 616. The third light source 612 emits a third beam of light 634 that is combined by a second beam combiner 636 with the second beam of light 630 prior to passing through the first beam combiner 632.

The processor may be configured with one or more instructions to perform any of the methods and/or any one of the steps and sub-steps of the methods or treatments described herein. The processor may comprise memory having instructions to perform the method, and the processor may comprise a processor system configured to perform the method for example. In many embodiments, the processor comprises array logic such as programmable array logic (“PAL”) configured to perform one or more steps of any of the methods or treatments described herein, for example.

The processor may comprise one or more instructions of a treatment program embodied on a tangible medium such as a computer memory or a gate array in order to execute one or more steps of a treatment method as disclosed herein. The processor may comprise instructions to treat a patient in accordance with embodiments described herein.

The processor may be configured with instructions to determine one or more locations of the limbus, and/or one or more locations of Schlemm's canal. In response to the determined location of limbus, for example, one or more locations of Schlemm's canal may be determined. The processor may be configured with instructions to determine a treatment pattern based on the one or more locations of the limbus and/or the one or more locations of Schlemm's canal. The treatment pattern may for example comprise a juxtacanalicular treatment pattern. The processor may be configured to deliver shrinkage energy to the sclera, cornea, or both in order to urge tissue near Schlemm's canal to move towards the treated tissue and dilate Schlemm's canal as described herein.

The optical delivery system may comprise one or more of the first light source, second light source, third light source, X-Y scanner, optional mask, or a heat sink. The energy may be directed by the optical energy delivery system to the eye or a hand held probe.

In many embodiments, the beams of light 614, 630, and 634 can be scanned onto the eye 602 at a specified X and Y position by the X-Y scanner 616 to treat the eye 602. The X-Y scanner can be configured to scan the combined light beams onto the eye 602 in a suitable treatment scan pattern, as previously described herein. An optional mask 618 can be used to mask the light applied to the eye 602, for example, to protect masked portions of the eye 602 while treating other portions as described herein. An optional heat sink 620 can be placed on the eye 602 during treatment to avoid heating specified portions of the eye 602, as described herein.

The system 600 can be used to apply light energy to the eye 602 in accordance with any suitable treatment procedure, such as the embodiments described herein. In many embodiments, the first light beam 614 has a first wavelength, the second light beam 630 has a second wavelength, and the third light beam 634 has a third wavelength. Each wavelength can be a different wavelength of light. Alternatively, at least some of the wavelengths can be the same. For example, in accordance with the embodiments described herein, the first light beam 614 can have a wavelength suitable to: cross-link an outer portion of the eye 602 and shrink an inner portion of the eye 602; shrink the inner portion and cross-link the outer portion concurrently; shrink the inner portion after the outer portion has been cross-linked; or any suitable combinations thereof. Alternatively, the first light beam 614 can have a first wavelength suitable to cross-link the outer portion of the eye 602, as described herein, and the second light beam 630 can have a second wavelength suitable to shrink the inner portion of the eye 602, as described herein. The third light beam 634 can have a third wavelength suitable to soften a portion of the sclera of the eye 602, as described herein. Any suitable combination of wavelengths of light for applying any combination of the treatments described herein, concurrently or separately, can be used.

The processor can be coupled to each of the light sources in order to selectively irradiate the eye with light having wavelengths within a desired range of wavelengths. For example, the first light source can be configured to emit light energy having wavelengths in a range from about 1.9 to 2.1 um, the 1/e attenuation depth can be in a range from about 200 to 300 ums, for example about 225 to 275 um. The second light source can be configured to emit light energy having wavelength in a range from about 1.3 to 1.55 um, the 1/e attenuation depth is within a range from about 350 to 450 um. The processor can be programmed with instructions to irradiate tissue with light energy appropriate for the effect at the desired treatment location. For example, the light source emitting light energy in the range from 1.9 to 2.1 um can be used to treat the cornea, and the second light source emitting light energy with wavelengths in the range from 1.3 to 1.55 um can be used to irradiate the sclera. The software may comprise instructions of a treatment table so as to scan the laser beam to desired treatment locations as described herein, for example.

The laser system 600 may comprise an OCT system 625, such as a commercially available OCT system. The OCT system may for example be a CASIA2 or CASIA SS-100 OCT scanner (TOMEY). The OCT system may for example be a commercially available OCT system such as one sold by Tomey, Heidelber, Visante, or Optovue. The OCT system can be coupled to the viewing optics and laser delivery system with a beam splitter 626. The viewing optics may for example comprise an operating microscope (such as one sold by Zeiss, Haag Streit, Leica, or Moller Weildel), a slit lamp, or other custom optics. The OCT system can be used to measure the eye in situ during treatment. For example, the OCT system can be used to generate OCT images as described herein in order to generate tomography of the eye to determine the location of target tissues, movement of target tissues, and stretching of target tissues as described herein. The OCT system 625 can be coupled to processor 604 and used to control the laser system with a feedback loop, for example.

The processor can be configured with instructions to scan the laser beam on the eye in accordance with the treatment patterns and parameters as described herein.

FIG. 8 shows another embodiment of a treatment system which may be used for any of the treatment methods described herein. The system may comprise a laser scanner which directs and scans laser energy from a continuous wave or pulsed laser to one or more locations on or inside the eye. The system may comprise a HIFU scanner which directs and scans HIFU energy from a HIFU transducer array to one or more locations on or inside the eye. The scanner may be coupled to a patient interface or patient coupling structure as described herein. The scanner may further be coupled to an imaging system, for example OCT or UBM, as described herein. The imaging system may be used to capture one or more images of the eye before, during, or after treatment as described herein. A processor or controller may be coupled to the energy source (e.g. HIFU transducer or laser) and the imaging system and be configured with instructions to scan the energy beam to a plurality of locations or in one or more patterns and image the tissue during treatment. The system may also comprise a display coupled to the processor that allows the user to visualize the tissue prior to, before, or after treatment. The display may show images which allow the user to see the tissue treated and plan the treatment. Images shown on the display may be provided in real-time and can be used to prior to treatment to allow the user to align the tissue and/or select a treatment zone or pattern to target. Identified target treatment zones may be input by the user to program the treatment depth, location, and pattern in response to the images shown on the display. The imaging system can be used to visualize movement of ocular structures during treatment in order to detect beneficial treatment effects. The processor can be configured with instructions to treat the eye with a first wavelength of ultrasound and to image the eye with a second wavelength longer than the first wavelength. The processor may alternatively or in combination be configured with instructions to treat the eye with HIFU or laser energy and to image the eye with an embedded imaging apparatus, for example an OCT probe or US imager. The processor coupled to the array can be configured with instructions to provide both ultrasound wavelengths from the array. The imaging apparatus may provide additional tissue feedback data in real-time, for example temperature or elasticity. The system as described herein may comprise an eye tracker as known to one in the art in order to generate real-time images of the eye in order to align or register the target treatment regions of the eye. Pre-treatment images can be measured and registered with real-time images obtained during treatment in order to track the location and orientation of the eye.

The glaucoma STEM treatment systems described herein may simultaneously provide imaging guidance, quantitative characterization of the tissue (for example measuring mechanical properties such as elasticity), and/or perform therapeutic tasks.

In some embodiments, the STEM treatment system described herein may comprise two or more lasers. The processor may be configured with instructions to treat the eye with a first wavelength of light at a first location (or plurality of locations) and a second wavelength of light at a second location (or plurality of locations). The treatment system described herein may comprise one or more lasers within a range of about 810 nm to about 6 um, for example about 810 nm, about 1.3 um to about 2.5 um, about 1.5 um to about 2.4 um, about 1.47 um, about 1.95 um, about 2.01 um, about 2.1 um, about 4 um to about 7 um, about 5 um to about 7 um, or about 6 um. Wavelengths on the lower end of the spectrum, for example an 810 nm or 1.47 laser may be used to treat the sclera. A 1.47 um laser may be about twice as tissue penetrating as a 2.01 um laser when equidosed. Wavelengths on the upper end of the spectrum, for example within a range of about 4 um to about 7 um may be used to directly target collagen and/or protein. A 6 um laser may be used to create scleral vacuoles for uveoscleral outflow enhancements for example. The system may optionally comprise a first laser with a first wavelength and a second laser with a second wavelength. The system may comprise a first laser with a first wavelength within a range of about 1.4 um to about 1.6 um and a second laser with a second wavelength within a range from about 1.9 um to about 2.3 um. The system may comprise a first laser with a first wavelength of about 1.47 um and a second laser with a second wavelength of about 2.1 um. The 1.47 um laser for example may be used to treat scleral tissue (or other recalcitrant, thick, dense, or opaque tissue) with deeper penetrance than the 2.1 um laser. The 2.1 um laser may for example be used to treat corneal tissue. The 2.1 um laser may for example be used to treat scleral tissue. In some instances, the sclera may be treated with both the 1.47 um laser and the 2.01 um laser at the same or different treatment locations, as the different wavelengths of light may produce different effects within the sclera which may be complimentary in designing a treatment plan. The processor may be configured with instructions to rapidly switch between the 1.47 um and 2.1 um lasers during treatment. In some embodiments, the system may comprise a first laser with a first wavelength of about 1.47 um and a second laser with a second wavelength of about 1.95 um and the processor may be configured with instructions to switch between the 1.47 um and 1.95 um lasers during treatment.

In some embodiments, the transducer array and the processor may be configured to provide a plurality of pulses to a plurality of separate treatment regions separated by a distance. A duty cycle of each of the plurality of separate treatment regions may comprise a duty cycle less than a duty cycle of the transducer array. The plurality of separate regions may comprise a first treatment region receiving a first plurality of pulses and a second treatment region receiving a second plurality of pulses, wherein the treatment alternates between the first plurality of pulses to the first region and the second plurality of pulses to the second region to decrease a duty cycle of each of the plurality of treatment regions relative to the duty cycle of the transducer array in order to decrease treatment time of the first region and the second region. The first treatment region may for example be a first annulus and the second treatment region may be a second annulus.

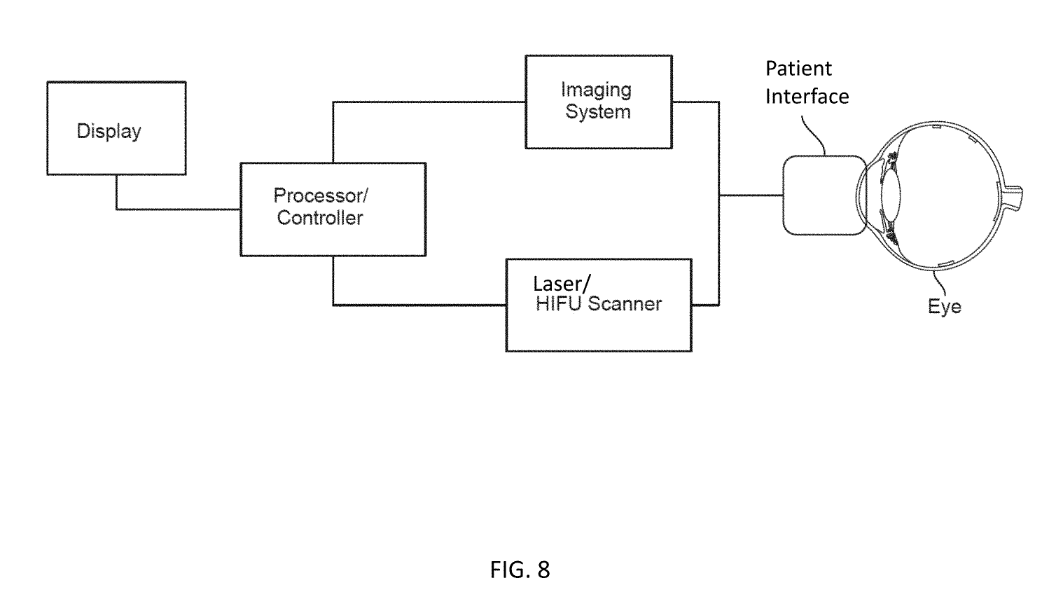

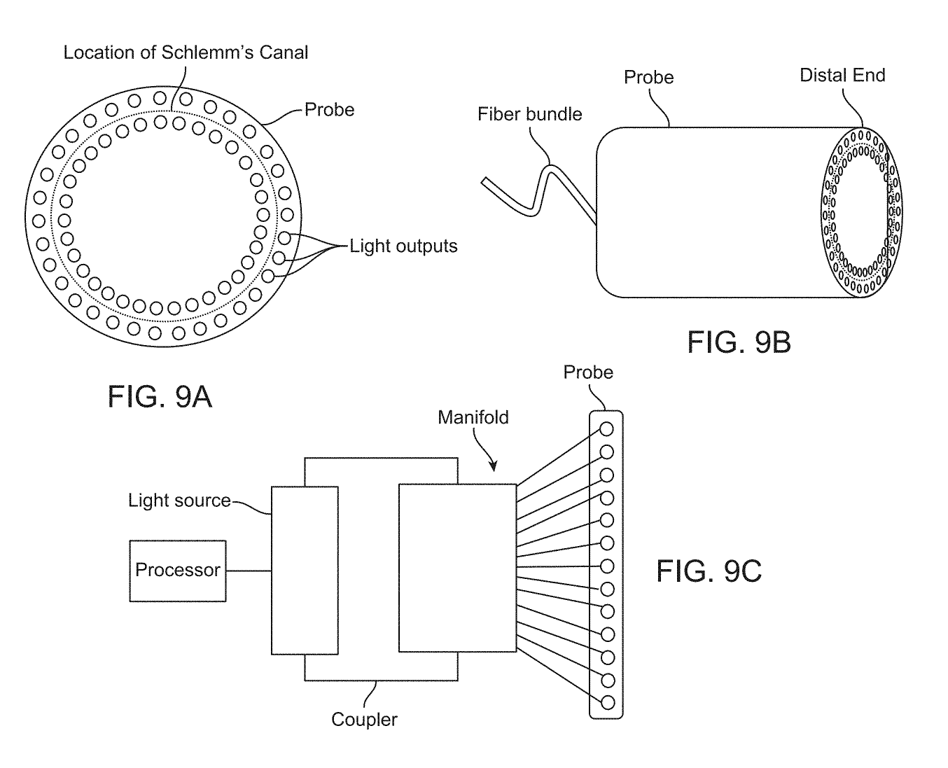

FIGS. 9A-9C show a handheld probe comprising a handpiece, in accordance with some embodiments. The handheld treatment probe may be used for any of the treatment methods and/or combined with components of any of the treatment systems described herein. FIG. 9A shows a plan view of the distal end of the handheld probe. FIG. 9B shows a perspective side view of the probe. FIG. 9C shows a schematic of the probe system, with a side plan view of the probe, coupled to a light source via a manifold in accordance with some embodiments.

The system may comprise a handheld probe which directs treatment energy to one or more locations on or inside the eye. In some instances, the distal end of the handheld probe may comprise a plurality of light outputs as shown in FIG. 9A. The light outputs may direct the treatment energy to one or more locations on or inside the eye. The light outputs may be oriented and/or spaced on the distal end of the handheld probe so as to target one or more region of the eye and/or avoid treatment in one or more region of the eye. For example, the light outputs may be arranged in so as to form two annuli on the distal end of the probe. The annuli may be spaced such that the outer annulus provides light energy to a portion of the eye that lies radially outward of Schlemm's canal and/or the limbus while the inner annulus provides light energy to a portion of the eye that lies radially inward of Schlemm's canal and/or the limbus. The dashed line in FIG. 9A represents an exemplary location of Schlemm's canal relative to the light outputs. In this way, the eye may be treated with the handheld probe in a juxtacanalicular manner as described herein. It will be understood by one of ordinary skill in the art that the light outputs may be arranged in any location and/or pattern on the distal end of the probe so as to provide treatment to the desired location(s) of the eye. For example, alternatively or in combination, an annulus of light outputs may be provide radially outward of the outer annulus shown in FIG. 9A at a desired location so as to treat the sclera to generate pores or vacuoles as described herein. Alternatively or in combination, the user may pattern the light outputs to avoid critical structures of the eye such as the vasculature.

The light outputs may for example comprise a plurality of light sources such as laser diodes or light emitting diodes configured to emit light at a wavelength suitable for treating the eye as described herein. Alternatively, the plurality of light sources may comprise openings in a mask configured to transmit light to desired treatment locations and block light at other treatment locations. In some embodiments the treatment probe comprises a diffractive optic, an axicon or lenses, as is known to one of ordinary skill in the art, configured to delivery energy to the plurality of treatment locations. Alternatively or in combination, the light outputs may transmit light towards the eye from a light source external to the handheld probe, for example from a light source via a fiber bundle as shown in FIG. 9B and/or from a laser light source via a coupler and a manifold assembly as shown in FIG. 9C. In some instances, the light outputs may be controlled as one with a processor as described herein. In some instances, the light outputs may be individually and independently controlled, for example in order to adjust the treatment pattern delivered to a patient's eye. The light outputs may provide continuous or pulsed light energy to the treatment location. The light outputs may be configured to deliver light energy at the same wavelength. Alternatively, the light outputs may be configured to deliver different wavelengths of light energy. For example, the outer annulus shown in FIG. 9A may comprise light at a wavelength of 1.48 μm to treat the sclera while the inner annulus may comprise light at a wavelength of 2.01 μm to treat the cornea.

Although reference is made to a probe comprising a handpiece configured for juxtacanalicular treatment, the probe can be configured in many ways to treat the eye, for example to treat regions of the sclera of the eye radially outward from Schlemm's canal and the limbus, and may be configured to avoid the cornea and limbus in some embodiments.

The handheld probe may be configured to be directly coupled with the patient eye. Alternatively or in combination, the handheld probe may be configured to be coupled to a patient interface or patient coupling structure as described herein.

It will be understood by one of ordinary skill in the art that the light outputs may be replaced by any source of treatment energy as described herein. For example, the light outputs may be replaced by radiofrequency electrodes or the like.

A system to treat glaucoma of an eye with the hand held energy probe may comprise an energy source, such as one or more of the laser light sources as described herein. The handpiece comprising the treatment probe is coupled to the energy source. The handpiece comprises an eye contacting surface to couple to the eye on the distal end of the probe, and a plurality of energy releasing elements disposed at a plurality of locations to release energy to the eye at a plurality of treatment locations within 2 mm of a Schlemm's canal of the eye. The distal end may comprise a concave shape such as a spherical shape or a conical shape to engage the eye near Schlemm's canal. The energy releasing elements may comprise electrodes or light outputs such as ends of optical fibers. The plurality of locations correspond to treatment locations located radially inward from the Schlemm's canal toward an optical axis of the eye and radially outward from the Schlemm's away from the optical axis of the eye or the center of the cornea as measured along an exterior surface of the eye.

The first plurality of locations may comprise a first plurality of locations located radially inward from a second plurality of locations. The first plurality of locations is arranged in a first annular treatment pattern to treat the eye radially inward from the Schlemm's canal and the second plurality of locations arranged to treat the eye with a second annular treatment pattern radially outward from the Schlemm's canal of the eye. The first annular treatment pattern generally corresponds to a first diameter within a range from about 10 mm to about 12 mm and the second annular treatment pattern corresponds to a second diameter within a range from about 12 mm to about 14 mm.

The plurality of energy releasing elements may comprise a plurality of optical fibers and the energy source may comprise a laser.

Alternatively or in combination, the plurality of energy releasing elements may comprise a plurality of electrodes and the energy source may comprise an electroporation energy source, a microwave energy source, a thermal energy source, an electrical energy source, an electrophoretic energy source, or a di-electrophoretic energy source.

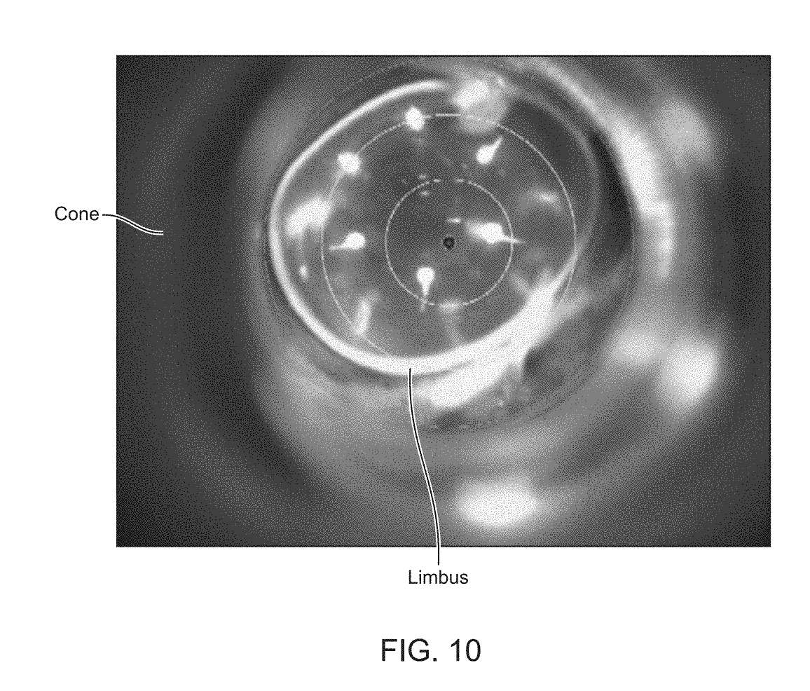

FIG. 10 shows an image of an ex vivo porcine eye taken with a camera after docking the patient interface and system to the eye. The limbus was clearly visible. As described herein, glaucoma treatment may be patterned relative to the location of the limbus. Patterning may be selected manually by the user (e.g. medical professional) or the patterns may be determined automatically (or semi-automatically) by the system based on an estimated location of the limbus, Schlemm's canal, or other fiducial of interest. The location of the limbus may be estimated manually by the user. The location of the limbus may for example be “tracked” automatically by the system using a camera and/or other imaging system such as OCT as described herein. The location of the limbus may comprise a complete, annular outline of the limbus or may comprise multiple locations along the limbus which may be used as reference points for determining the shape of the limbus and/or where treatment should occur (i.e. an incomplete outline of the limbus). Treatment may be patterned to include or avoid the limbus as desired by one of ordinary skill in the art.

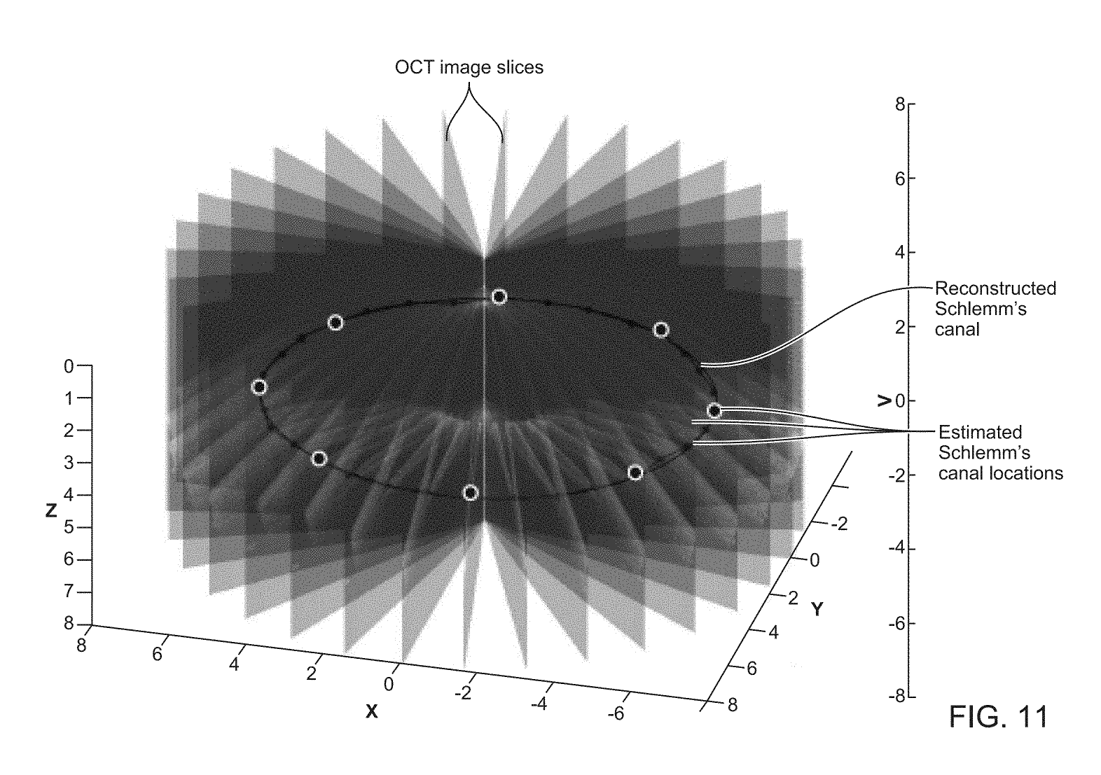

Glaucoma treatment may be patterned relative to the location of Schlemm's canal. Identification of one or more locations of the limbus as described herein may be used to estimate one or more locations of Schlemm's canal. The limbus may be used as a surrogate for Schlemm's canal as they can be located at measurable or known distances from one another. The location of Schlemm's canal with respect to the limbus may vary systematically with age and/or IOP. For example, Schlemm's canal may be further away from the limbus in younger eyes than in older eyes. Schlemm's canal may be further away from the limbus in patients with increased IOP compared to patients with normal IOP. Such variations may be taken into account when patterning treatment. For example, treatment may be patterned further out from the limbus in an older patient than in a younger patient to account for the difference in location of Schlemm's canal with reference to the limbus. In some instances, the limbus and Schlemm's canal are roughly the same position radially within the eye and the position of the limbus may be roughly the position of Schlemm's canal. Alternatively or in combination, one or more locations of Schlemm's canal may be estimated from one or more OCT slices. For example, a single OCT image taken through the center of the eye may be used to identify two locations of Schlemm's canal (one on either side of the eye) and the treatment locations/pattern may be determined in response to the two Schlemm's canal locations identified. In some instances, multiple OCT images may be taken at different angles relative to the center of the eye and a plurality of Schlemm's canal locations may be identified and used to estimate the shape of Schlemm's canal. FIG. 11 shows one such imaging scheme which may be used to estimate the shape of Schlemm's canal. Multiple OCT images may be taken across the center of the eye at varying angles and the one or more location of Schlemm's canal may be estimated from each image. The locations (and optionally the images as shown) may then be used to estimate the shape of Schlemm's canal using partial 3-D reconstruction.

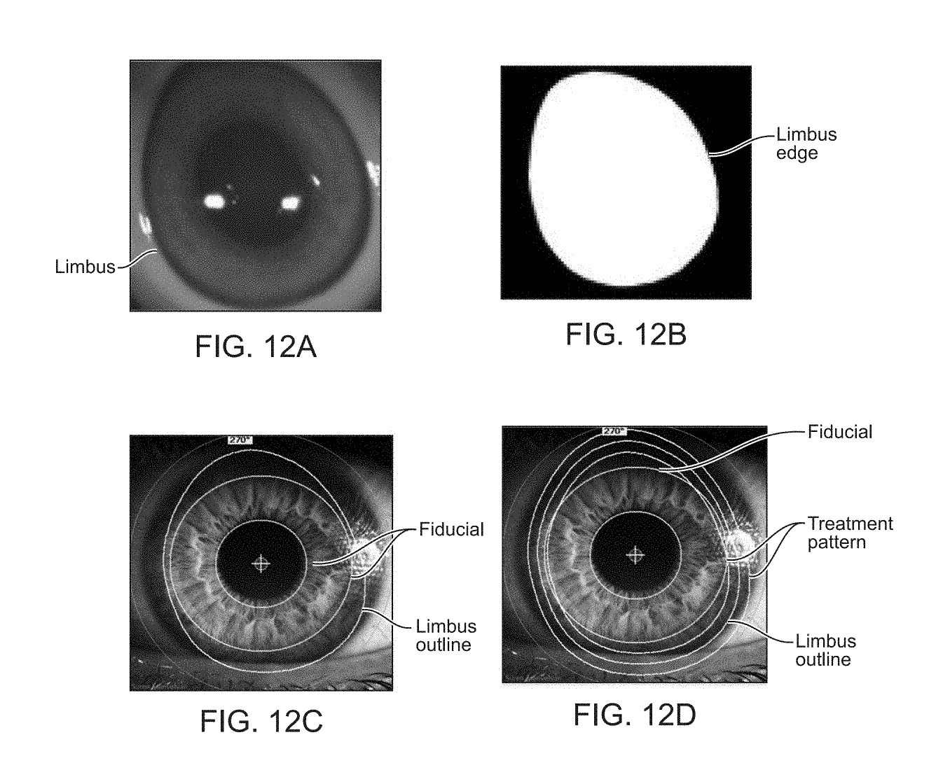

In some instances, the location and/or shape of the Schlemm's canal may be estimated in response to a plurality of limbus locations of the eye. As an example, an image (e.g. an anterior image of the eye) including the limbus may be acquired (e.g. as shown in FIGS. 10 and 12A). The locations of the limbus, or plurality of limbus locations may be determined, based on the image of the eye. For example, by detecting changes in intensity in the anterior image (e.g. across the image, over a series of images, etc), the location of a plurality of limbus locations may be determined. In some instances, one or more processors may be utilized to analyze the image to determine the limbus locations. Based on the plurality of limbus locations, a plurality of Schlemm's Canal locations may be estimated substantially as described throughout.

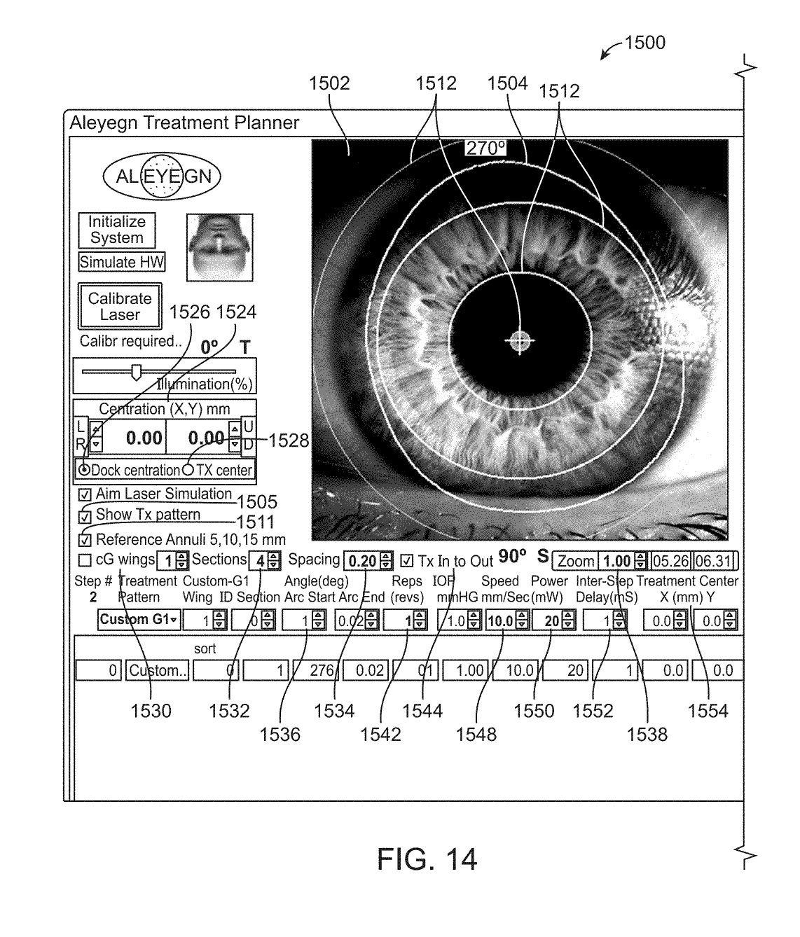

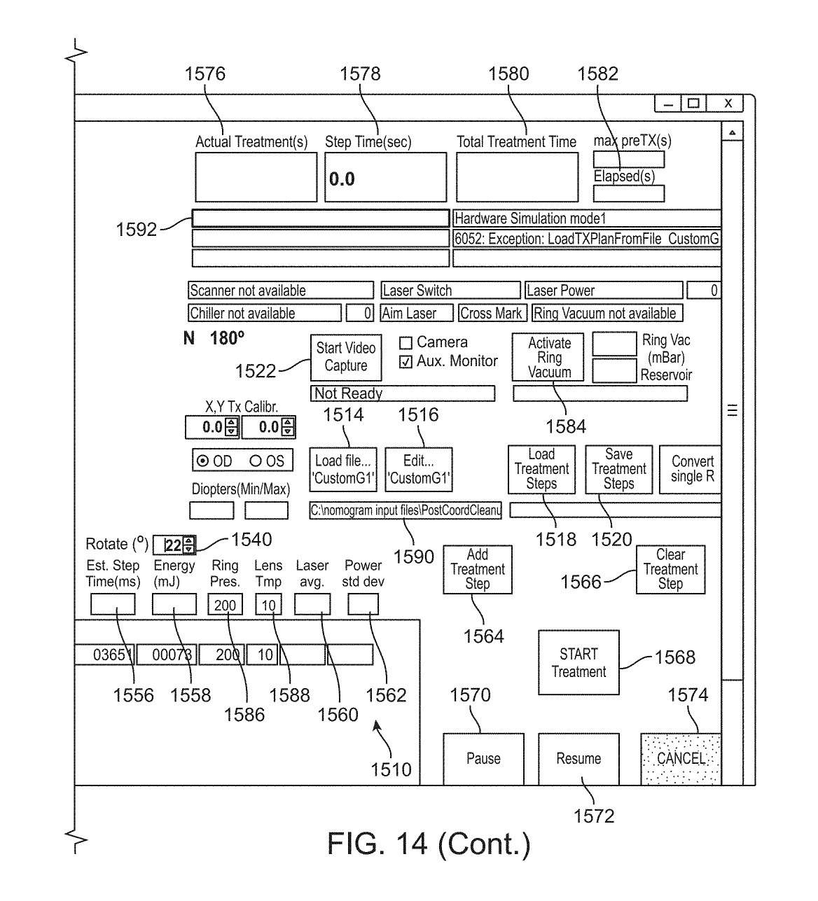

FIGS. 12A-12D show an exemplary process for generating a treatment pattern based on one or more locations of the limbus. FIG. 12A shows an anterior image of an eye taken with a front camera of a CASIA2 OCT system. The anterior image of the eye may be transferred to the processor for detection of the limbus edge. FIG. 12B shows the image after processing to determine the boundaries (edges) of the limbus. The portion of the eye within the limbus has been colored white while the portion of the eye outside the limbus has been colored black. This black and white image may then be used to generate X-Y coordinates of the edge of the limbus. The processor may then use the X-Y coordinates to generate an outline of the limbus which may be overlaid onto a real-time image of the eye shown on the display as shown in FIGS. 12C and 14. The X-Y coordinates may be registered with the real-time image of the eye such that the limbus shown on the display and the X-Y coordinate generated limbus outline are co-aligned. The may also display fiducials with reference to the center of the eye to aid in centration of treatment, for example circles displayed radially outward every 5 mm from the center of the eye. The processor may further use the X-Y coordinates and/or the generated outline of the limbus in order to determine a treatment pattern, for example a series of juxtacanalicular treatments patterned radially inward and/or outward of and shaped like the limbus as shown in FIGS. 12D, 15, and 16-21. Note that the images of the eye shown in FIGS. 12C-12D, and 14-21 over which an outline of the limbus and/or treatment patterns is shown, are images of a representative eye for visualization purposes and not real-time images of the eye used to generate the limbus outline/treatment nomogram and therefore the outline and the limbus on the image do not co-align in these examples. It will be understood by one of ordinary skill in the art that during use the limbus outline and/or treatment patterns may be registered to one another (i.e. co-aligned) such that the outline and real-time location of the limbus overlap.

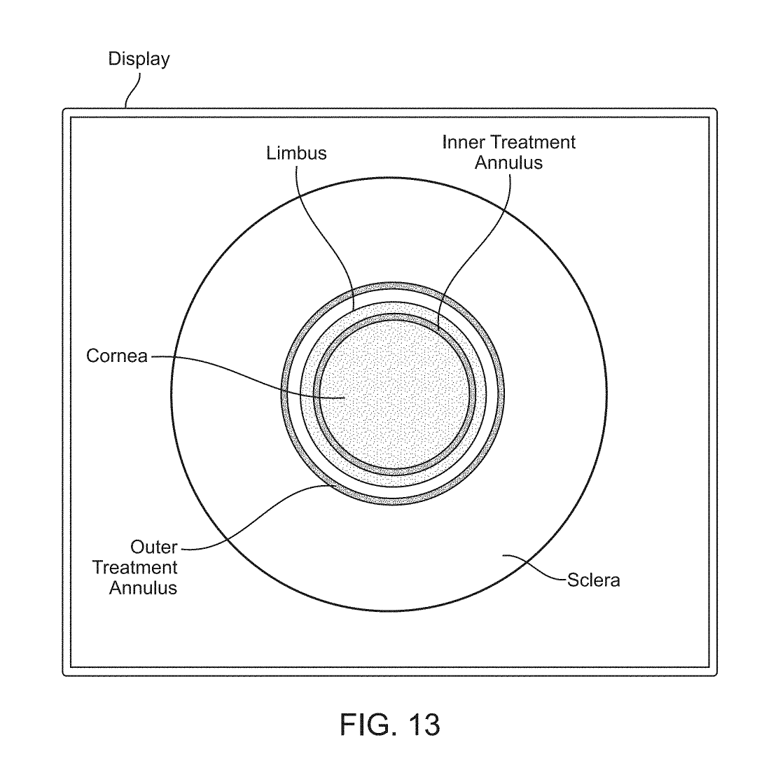

FIG. 13 shows a schematic of a display for use in directing treatment to targeted treatment zones (also referred to herein as targeted treatment regions). The glaucoma system described herein may allow for pre-treatment planning and/or treatment of a tissue in an image-guided manner. Treatment locations and patterns may for example be input by a user in response to an image shown on the display. Alternatively or in combination, the treatment locations of patterns may be determined in response to an image of the eye or a plurality of OCT images of the eye, for example in response to an estimated location of Schlemm's canal or the limbus as described herein. The image shown on the display may be obtained pre-operatively or in real-time prior to or during treatment. The image shown on the display may comprise a real-time anterior image of the eye captured by an operating microscope or camera coupled to the system. The image shown on the display may comprise one or more OCT image slices of the eye. Targeted treatment pattern(s) and/or location(s) may be selected by a user or operator in response to the image displayed. The user may input the desired treatment patterns so as to provide the processor with instructions to scan and/or pulse the energy beam to the targeted treatment locations. The user may for example input the desired treatment patterns using a touch-screen to select the target zones directly on the displayed image or by using a joystick or mouse to point a cursor at the target locations. Alternatively or in combination, the processor may be configured with instructions to generate one or more treatment locations and/or one or more treatment patterns based on an image of the eye.

For example, the glaucoma STEM adjustment system may be used to target treatment to straddle Schlemm's canal. Real-time anterior image(s) and/or OCT image slices of the eye may be acquired and displayed for the user (for example a doctor) to view. In some instances, the treatment may be located at a predetermined radial distance relative to the center of the eye. Alternatively, treatment may be located with respect to one or more fiducial markers such as the limbus, the anterior chamber width, Schlemm's canal, the iris, the trabecular meshwork, or any combination thereof. For example, a fiducial such as the limbus or Schlemm's canal may be identified by the user and the treatment may be patterned by the user directly onto the image of the eye using a touch screen in response to the location of the limbus. Alternatively, the limbus or Schlemm's canal may be selected using a touch screen and the treatment may pattern may be generated by the processor in response to the location of the limbus. The processor may then direct the energy source to deliver treatment energy to the treatment locations, for example an inner treatment annulus and an outer treatment annulus straddling the limbus/Schlemm's canal. In some instances, the processor may be configured with instructions to direct energy into the eye with an annular pattern. The annular pattern may in some instances comprise an inner dimension located radially inward from the Schlemm's canal and an outer dimension radially outward from the Schlemm's canal.

The processor may be configured with instructions to receive user inputs to define the plurality of targeted tissue locations on the image of the eye prior to treatment with the treatment energy. The processor may be configured with instructions to register the plurality of target tissue locations defined prior to treatment with a real time image of the eye acquired during the treatment and to show the target tissue locations of the eye in registration with the real time image of the eye. The imaging system (e.g. OCT, UBM, US, etc.) may be aligned with the energy source, for example a laser or ultrasound transducer array. The processor may comprise instructions to direct the treatment energy to the plurality of treatment locations in response to registration of the real time image of the eye with the image of the eye in response to movement of the eye.

The processor may be configured to scan the energy beam to a plurality of locations. An ultrasound transducer array may comprise a phased array configured to scan an ultrasound beam to the plurality of locations. The system may optionally further comprise an actuator coupled to the ultrasound array to scan the ultrasound beam to the plurality of locations. A laser energy source may be coupled to a scanner and configured to scan a laser beam to the plurality of locations. The system may optionally further comprise an actuator coupled to the laser to scan the laser beam to the plurality of locations.

The processor may be configured with instructions to generate the HIFU beam comprising a plurality of pulses. Each of the plurality of pulses may comprise at least one acoustic cycle. Each pulse of the plurality of pulses may be separated from a subsequent pulse of the plurality of pulses by a time within a range from about 1 microsecond to about 1000 microseconds in order to provide a duty cycle of no more than about 5 percent (%) to a target tissue region.

The processor may be configured with instructions to generate a laser beam comprising a plurality of pulses. Each pulse of the plurality of pulses may be separated from a subsequent pulse of the plurality of pulses by a time within a range from about 1 microsecond to about 1000 microseconds in order to provide a duty cycle of no more than about 5 percent (%) to a target tissue region.

The system may for example comprise a phased array transducer, a one dimensional phased array transducer, a two dimensional phased array transducer, a translation stage, an X-Y translation stage, an actuator, a galvanometer and a gimbal. Alternatively or in combination, the system may comprise a handheld probe.

The treated pattern may not produce an optically visible artifact to a patient viewing with the eye for a period of time post-treatment within a range from about one week post-treatment to about one month post treatment.

In some instances, the processor may be configured with instructions to estimate a plurality of locations of Schlemm's canal of the eye. Optionally, the processor may be configured to generate a plurality of treatment locations for the eye in response to the plurality of locations of Schlemm's canal of the eye. The plurality of treatment location may in some instances be located within 2 mm radially of the Schlemm's canal. Optionally, the plurality of treatment locations may be located radially inward from the Schlemm's canal toward an optical axis of the eye or radially outward from the Schlemm's away from the optical axis of the eye as measured along an exterior surface of the eye.

The processor may be further configured with instructions to deliver energy to the treatment locations. The processor may in some instances be further configured with instructions to shrink collagenous tissue near the Schlemm's canal. In other instances, the processor may be configured with instructions to treat scleral tissue of the eye to provide vacuoles in the sclera and increase outflow through the sclera in order to lower intraocular pressure. Optionally, the processor may be configured with instructions to treat the eye in order to increase an angle of the eye, dilating and stretching one or more of the trabecular meshwork or Schlemm's canal, increasing porosity of the sclera and dilating the perilimibic sclera.

In some instances, the processor may be configured with instructions to treat the eye with a plurality of patterns. For example, the processor may be configured with instructions to treat the eye with a first pattern and a second pattern. In some instances, the first pattern may be utilized to treat glaucoma of the eye and a second pattern may be utilized to treat a refractive error of the eye. The first and second patterns may be as substantially described throughout.

The processor may be configured with instructions to apply a total amount of energy substantially as described throughout. For example, the processor may be configured with instructions to apply a total energy within a range from about 4 J to about 90 J. Optionally, the energy directed to the eye may have a total energy within a range from about 5 J to about 50 J. Optionally, the energy directed to the eye may have a total energy equal to or less than about 1 J, 2 J, 5 J, 10 J, 15 J, 20 J, 25 J, 30 J, 35 J, 40 J, 45 J, 50 J, 60 J, 70 J, 80 J, 90 J, 100 J, 120 J, or 150 J.

FIGS. 14-15 and 16-21 show optional, exemplary graphical user interfaces which may be displayed on the display to the user of the STEM system described herein. The graphical user interface may (GUI) be configured to allow the user to adjust the treatment parameters and provide instructions to the processor as described herein.

The GUI may provide the user with the ability to generate treatment patterns and/or adjust pre-generated treatment patterns (such as juxtucanalicular treatment patterns generated based on the outline of the limbus as described herein). Pre-generated treatment patterns may be overlaid onto a real-time image of the eye as described herein. In some instances, for example, it may be desirable to open Schlemm's canal and a juxtacanalicular treatment pattern may be displayed to the user. The user may decide to keep or adjust the treatment pattern generated. In some instances, the user may combine treatment patterns for multiple indications—for example by adding a pars plana treatment pattern to generate scleral vacuoles to a juxtacanalicular treatment pattern. Any of the treatment patterns described herein may be combined as desired by one of ordinary skill in the art.

Parameters which may be adjusted by the GUI include rotation of the treatment pattern and X-Y centration of the treatment pattern. In many instances, X-Y centration of the treatment pattern occurs automatically upon registration of the real-time image and the X-Y coordinates generated from a pre-operative anterior image of the eye, such as an OCT image of the eye, based on the location of one or more fiducials. Additionally, the speed, pulse frequency, power, beam diameter, and number of repetitions performed by the laser may be adjusted by the user based on the desired treatment results. The spacing of treatment arcs, location of treatment arcs, diameter of treatment arcs, and/or the shape of the treatment arcs may be adjusted by the user in order to generate the desired treatment pattern. In some instances, a treatment arc may be a 360 degree treatment. In some instances, a treatment arc may be less than 360 degrees. The processor may be configured to compute and display to the user the amount of time of treatment, the amount of energy to be deposited, the power used by the laser, the amount of time elapsed, the amount of time left in treatment, and/or the temperature of the cooling structure (e.g. contact lens) during treatment in order to monitor the progress and/or adjust the treatment parameters in real-time.

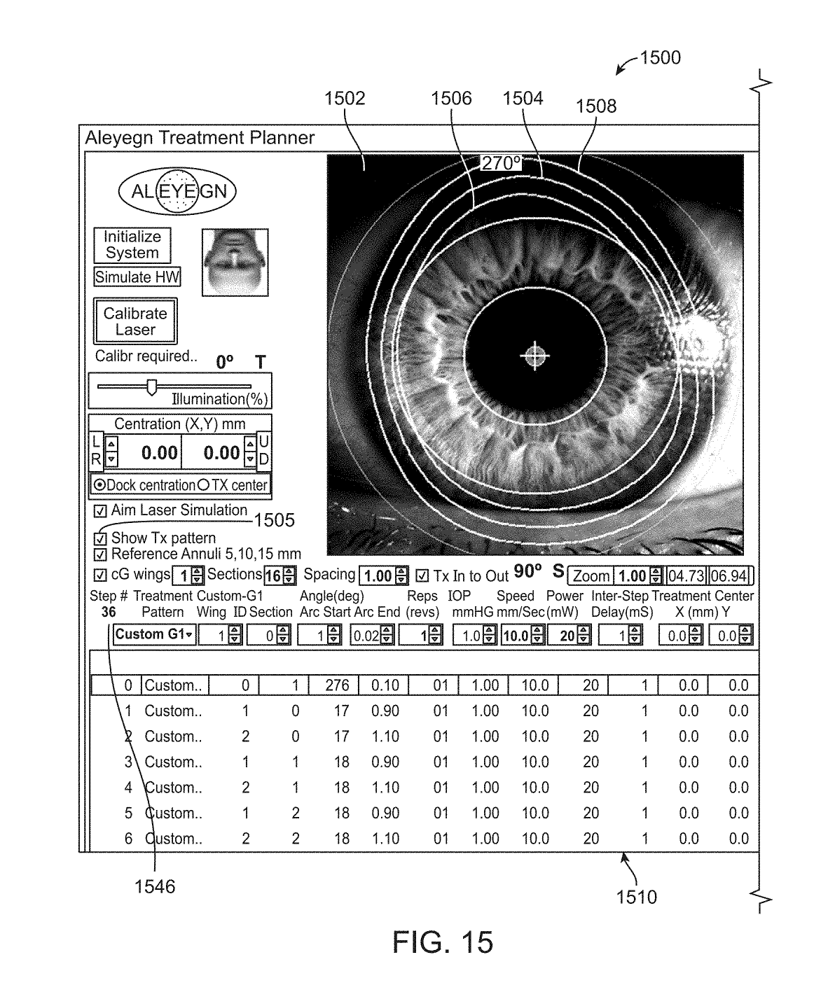

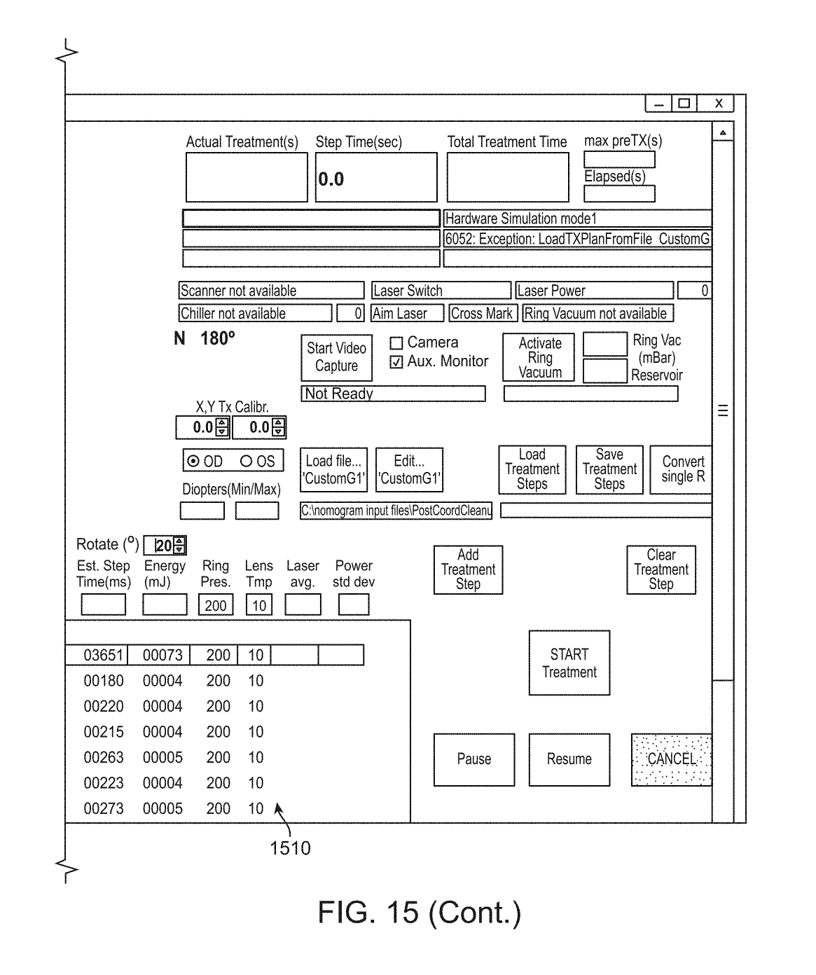

FIGS. 14 and 15 show a graphical user interface 1500 which may be displayed on the display to the user of the STEM system described herein. The graphical user interface may (GUI) 1500 be configured to allow the user to adjust the treatment parameters and provide instructions to the processor. The GUI 1500 may be configured to allow the user to plan the treatment (e.g. set up the treatment pattern, parameters, etc.) prior to treatment. The GUI 1500 may display an anterior image of the eye 1502 to the user prior to, during, or after treatment as described herein. For example, as shown in FIG. 14, the anterior image of the eye 1502 may be displayed with an overlaid outline of the limbus 1504 generated as described herein. In some instances, it may also be desirable to display the treatment location 1506, 1508 on the anterior image of the eye 1504 as shown in FIG. 15. In some instances, it may be desirable to display reference 1512 annuli to the user, for example concentric circles at 1 mm, 5 mm, 10 mm, and 15 mm from the center of the eye. Such overlays 1504, 1506, 1508, 1512 may be configured to remain on the real-time anterior image of the eye 1502 displayed during treatment so that the user may monitor treatment progress and accuracy. In some instances, it may be desirable to remove the one or more of the overlays 1504, 1506, 1508, 1512 when treatment begins such that treatment may be monitored without being obscured by the overlay. One or more of the overlays may be removed manually prior to starting treatment. Alternatively or in combination, one or more of the overlays may be manually chosen to be removed when the start treatment button 1568 is selected. Alternatively or in combination, one or more of the overlays 1504, 1506, 1508, 1512 may automatically be removed when the start treatment button 1568 is pushed to begin treatment of the eye. The anterior image of the eye 1502 may for example be a static image or a video of the eye. The anterior image of the eye 1502 may for example be a three-dimensional image of the eye.

In use, as an example, a user may upload an anterior image of the eye 1502 to the GUI 1500 or start the camera to generate an anterior image of the eye 1502 (e.g. a real-time video stream or static two dimensional or three-dimensional image of the eye of a patient coupled to the system with a patient interface as described herein) using the start video capture button 1522. For example, the user may push the start video capture button 1522 to begin real-time video monitoring of the docked patient eye. The user may then use the GUI to generate a treatment pattern based on the video of the docked patient eye. Alternatively or in combination, the user may push the start video capture button 1522 to start the video camera then push it again to stop the camera and leave a static image of the patient's eye displayed for the user to work from. The GUI may be used to activate the vacuum on the patient interface ring using the activate ring vacuum 1584 when the patient has been docked to the patient interface.

The anterior image of the eye 1502 may be loaded with a fiducial marker like the limbus outline 1504 described already drawn on it. Alternatively or in combination, the limbus outline 1504 may be loaded separately of the anterior image of the eye 1502 using the load file button 1514. The x-y coordinates used to generate the limbus outline 1504 may be generated from the anterior image of the eye 1502 using the process described in FIG. 8 for example. In some instances, the user may want to update or adjust the limbus outline 1504 previously generated, for example to adjust centration, size, etc., and may make adjustments using the edit button 1516. Pushing the edit button 1516 may cause the parameter adjustment buttons to modify the limbus outline 1504 instead of the treatment pattern. The name 1590 of the file which contains the limbus outline 1504 may be displayed to the user by the GUI 1500. Alternatively or in combination, the user may not upload a limbus outline 1504 and may manually pattern the treatment based on a visual inspection of the location of the limbus. In some instances, the user may choose to apply reference annuli 1512 to the anterior image of the eye 1502 using the reference annuli 5, 10, 15 mm button 1511.

After the image 1502 has been displayed with the limbus outline 1504 (if desired) and/or reference annuli 1512, the user may then begin planning the treatment. Previously generated treatment steps may be uploaded using the load treatment step button 1518. Treatment plans generated for future treatments may be saved using the save treatment steps button 1520. While a juxtacanalicular treatment pattern is shown in FIG. 15, it will be understood by one of ordinary skill in the art that any of the treatment patterns and parameters described herein may be planned for using the GUI.

In the case of juxtacanalicular treatment, a new treatment plan may include at least one limbus-shaped inner annulus 1506 positioned radially inward of the limbus outline 1504 and at least one limbus-shaped outer annulus 1508 positioned radially outward of the limbus outline 1504. The user may press the show treatment (“Tx”) pattern button 1505 to display/remove the treatment annuli 1506, 1508 on the anterior image of the eye 1502 as desired. The annuli 1506, 1508 may be generated using cG wings button and input field 1530. The button 1530 may be used to apply the wings (i.e. a pair of inner and outer annuli) 1506, 1508 while the input field 1530 may be used to adjust the number of wing pairs 1506, 1508. While only a single inner annulus 1506 and a single outer annulus 1508 are shown, it will be understood by one of ordinary skill in the art that additional annuli may be added to the treatment plan as described herein by increasing the number of wing sets in the GUI.

The spacing/step size between annuli (in mm) may be adjusted using the spacing input field 1534. When no reference fiducial like the limbus outline 1504 is used, or when treatment is patterned without reference to the limbus, the cG wings button 1530 (or another button not shown) may be used to pattern treatment annuli on the eye, for example at a selected distance from the center of the eye.

The rotation of the treatment annuli may be adjusted using the rotate input field 1540. The angle of rotation may be configured to be consistent with conventional reference angles used by a physician when looking at the eye through an operating microscope, as will be understood to one of ordinary skill in the art. The temporal (“T”) direction may be at 0°, the nasal (“N”) direction may be 180°, the superior (“S”) direction may be at 90°, and the inferior (“I”) direction may be at 270° (shown here oriented towards the top of the display).

The center of the treatment pattern may be adjusted by changing the X and Y coordinates in the Centration (X,Y) mm input field 1524 when the Tx center button 1528 is selected. The center of the reference annuli 1512 may be adjusted by changing the X and Y coordinates in the Centration (X,Y) mm input field 1524 when the dock centration button 1526 is selected. Centration of the reference annuli 1512 may occur automatically or may be done by the user manually as described herein.

The user may select the order of treatment of the annuli using the GUI. For example, the user may select the Tx in to out button 1544 may be selected in order to include instructions to the processor that the annuli are to be treated in order from the innermost annulus 1506 to the outermost annulus 1508. Alternatively or in combination, the user may use the GUI to direct treatment to occur in a “mark and jump” pattern between treatment annuli, to occur with alternating rings being treated to generate a suspension effect, or to occur with another pattern as described herein or otherwise desired by one of ordinary skill in the art.

The size of the treatment annuli 1506, 1508 may be adjusted using the zoom input field 1538.

The treatment annuli 1506, 1508 may be made up of one or more treatment arcs. For example, the annuli 1506, 1508 may be each comprise 4 treatment arcs such that the treatment for each annulus 1506, 1508 is broken up into 4 pieces (e.g. 4 quarters of a circle for example), each piece being a “step” in the plan. The number of sections/arcs may be selected by the user using the sections input field 1532.

The user may adjust the amount of time (i.e. the delay) between steps using the inter-step delay (ms) input field 1552. In some instances it may be beneficial to incorporate a delay between treatment steps, for example to avoid overheating of the tissue when overlapping treatment patterns are used or to account for the time necessary to switch between laser wavelengths when multiple wavelengths of light are used for treatment as described herein.

The user may select the number of times for each annulus to be treated (e.g. the number of full revolutions or repetitions along the annulus) using the reps (revs) input field 1542.

The user may adjust the speed of the laser using the speed mm/sec input field 1548. The user may adjust the power of the laser using the power (mW) input field 1550.

Once the displayed treatment pattern and treatment parameters have been planned by the user, the user may press the add treatment step button 1564 to display the calculated treatment steps in the treatment step menu 1510. The number of steps 1546 may be displayed. Additional steps may be added by generating new patterns/parameters and selecting the add treatment step button 1564 to add the next set of treatment steps to the menu 1510. The clear treatment steps button 1566 may be used to clear treatment steps from the menu 1510, for example by clearing highlighted (or otherwise indicated) treatment steps selected by a user.

The treatment step menu 1510 may display a variety of information about each step of treatment based on the treatment parameters and patterns selected by the user during treatment planning. For example, information about which wing and which arc on said wing is being treated may be displayed in columns in the menu 1510. The start and end angles of the treatment arc may be displayed in columns 1536. The user-selected repetition number 1542, the selected scan speed 1548, the selected laser power 1550, the selected inter-step delay 1552, and the treatment center 1554 may also be displayed in columns. Additional information may be calculated based on the treatment planned. Such information may be calculated/generated once the add treatment step button 1564 is pressed and displayed by the GUI. Calculated and displayed information may include an estimate of the time to complete each step (in ms) 1556 and/or the amount of energy (in mJ) 1558 delivered in each step. The GUI may also be configured to display the pressure of the patient interface ring 1586 and/or the temperature of the cooling structure (e.g. cooled contact lens) in contact with the patient's eye as described herein.

Treatment may begin when the user selects the START treatment button 1548. The user may also use the GUI to pause treatment with the pause button 1570, resume treatment with the resume treatment button 1572, and cancel treatment with the cancel treatment button 1574. The system may treat the eye until it finishes the final treatment step, or until the pause or cancel buttons 1570, 1574 are pressed, at which point the system may automatically stop the laser and return full control to the GUI 1500.

While treatment is underway, a progress bar 1592 may be displayed to show the user how far along treatment is. Alternatively or in combination, the amount of time elapsed for each step 1576, the amount of time elapsed in total 1582, and/or the estimated step treatment time remaining 1578 may be displayed. Once finished, the total treatment time field 1580 may display the total amount of time it took to complete the planned treatment. After each step, or after completing the treatment, the average laser power delivered per step 1560 and the standard deviation 1562 may be displayed in the menu 1510.

Not all of the elements in FIGS. 14-15 are labeled in order to make the illustration less cluttered and easier to see. While the GUI shown is configured to be used with a laser system, it will be understood by one of ordinary skill in the art that the GUI may be modified to be used with other energy sources or treatment systems as described herein.

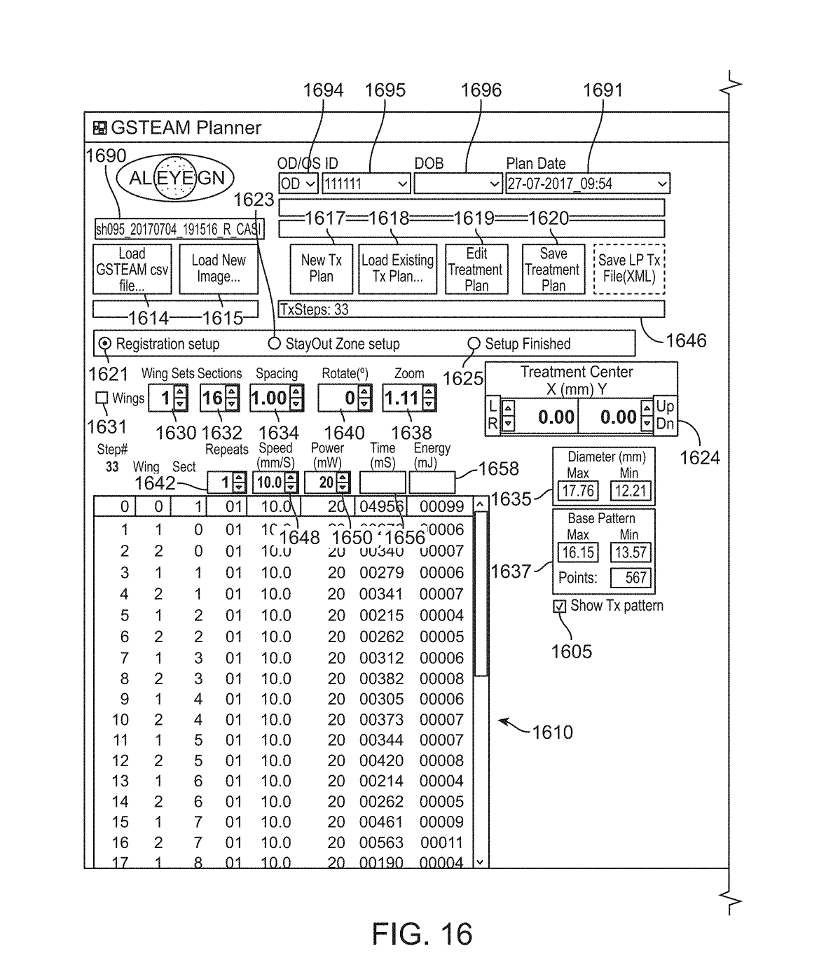

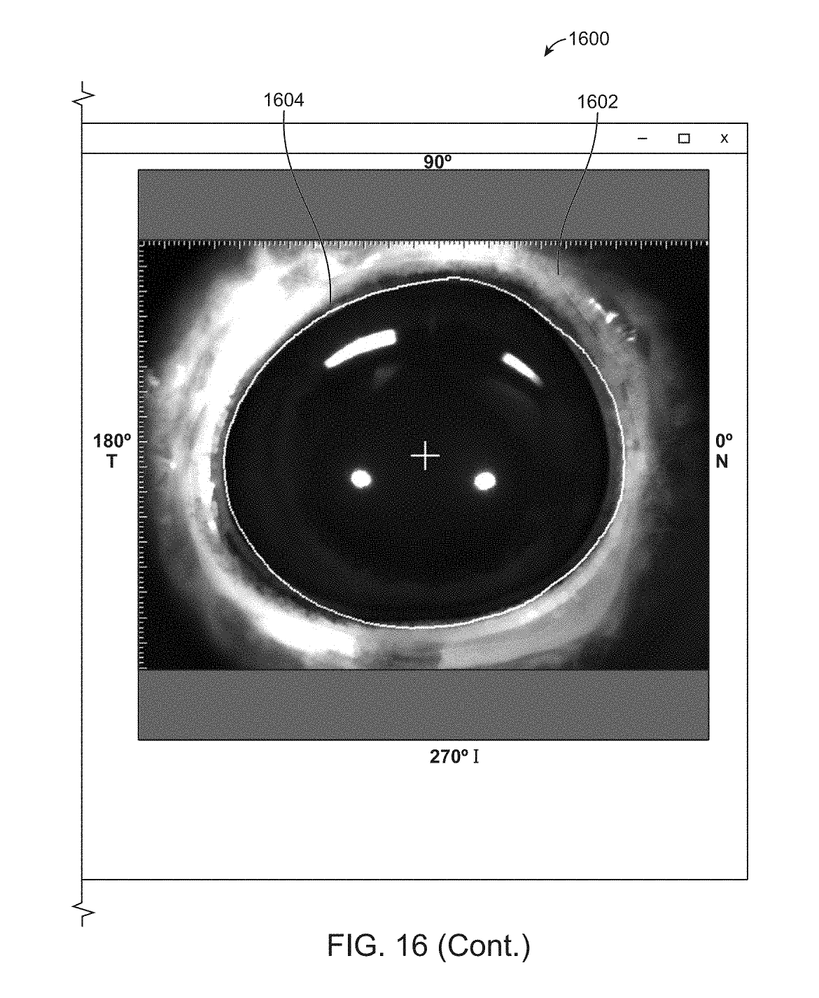

FIGS. 16-21 show another GUI 1600 which may be displayed on the display to the user of the STEM system described herein.

FIG. 16 shows a screen capture of the GUI 1600 on the registration setup page with an anterior image of the eye 1602 uploaded and displayed to the user with a limbus outline 1604 displayed thereon and generated as described herein

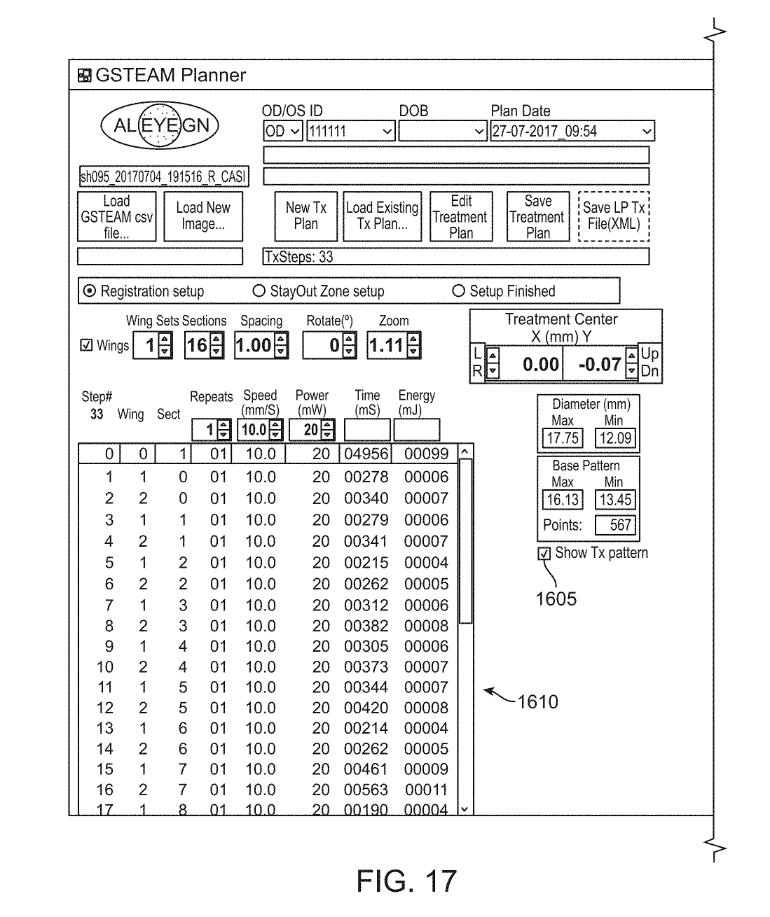

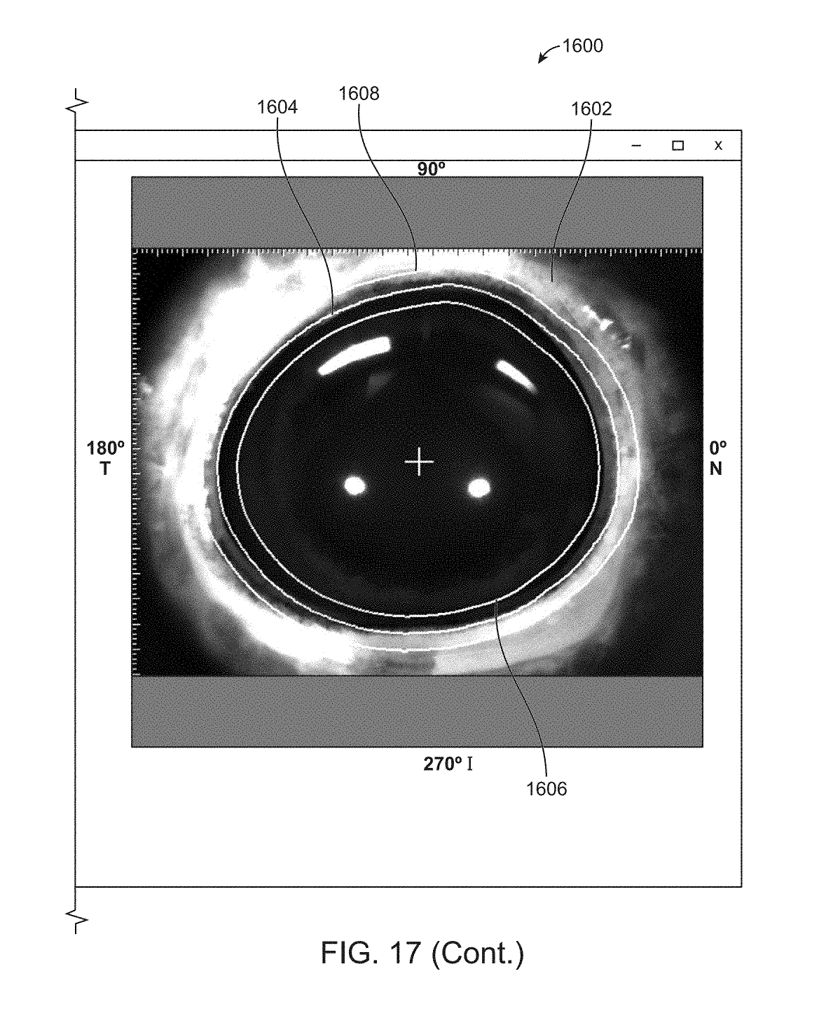

FIG. 17 shows a screen capture of the GUI 1600 on the registration setup page with two treatment annuli 1606, 1608 displayed on either side of the limbus outline 1604 overlaying the anterior image of the eye 1602.

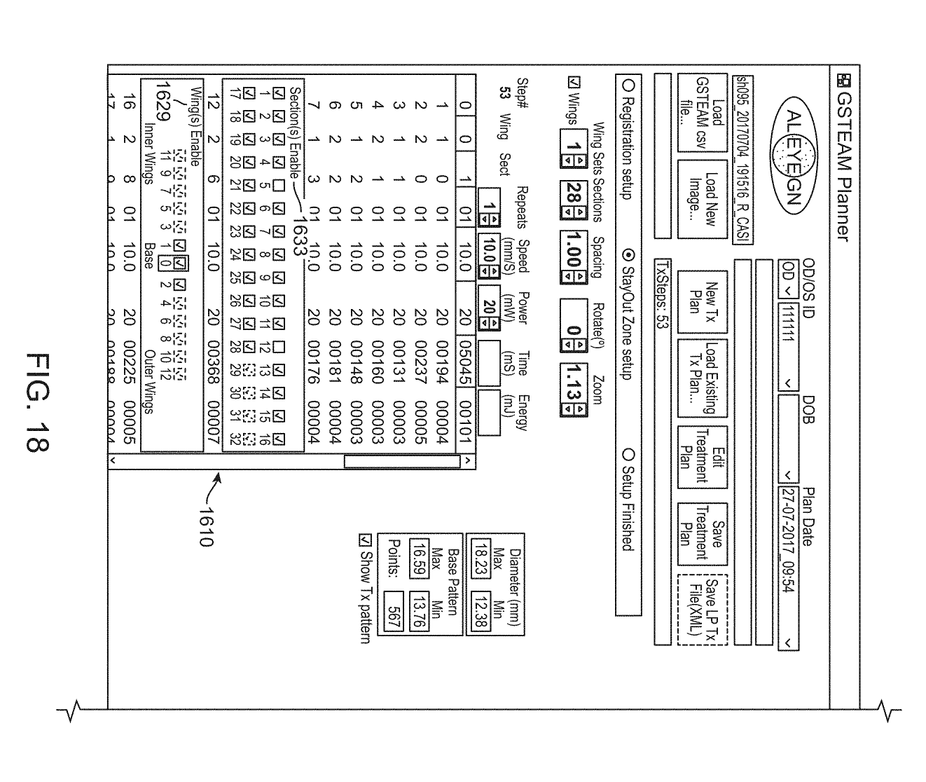

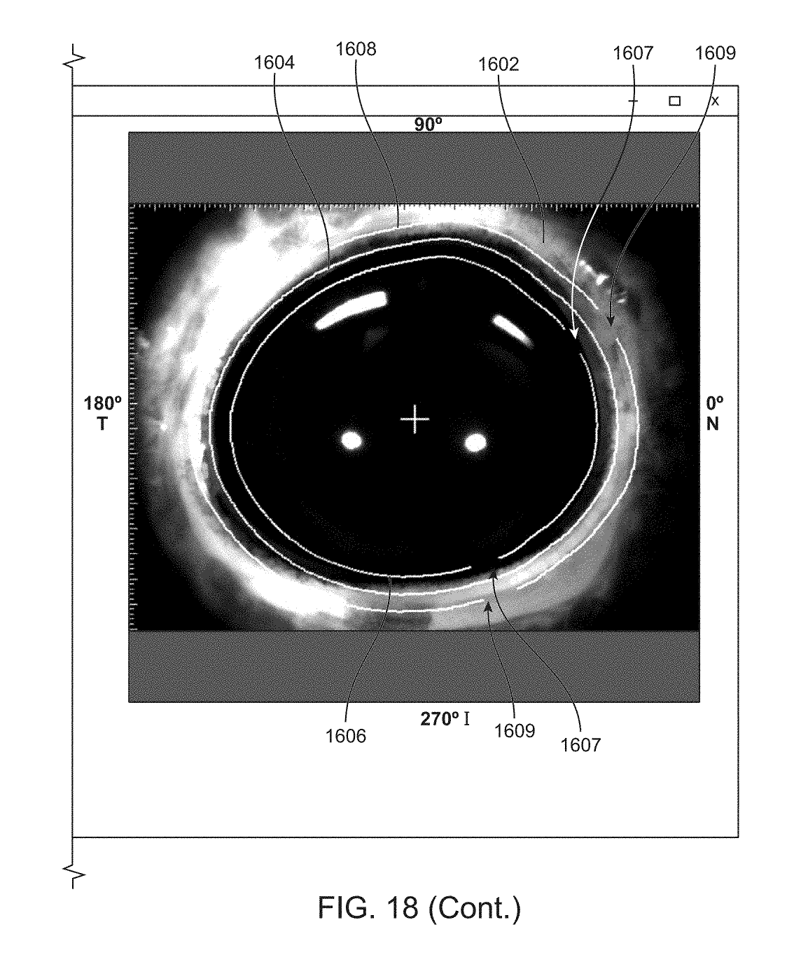

FIG. 18 shows a screen capture of the GUI 1600 on the stayout zone setup page with a section disabled from each of the inner and outer annuli 1606, 1608. Disabled sections 1607, 1609 appear as gaps in the treatment annuli 1606, 1608.

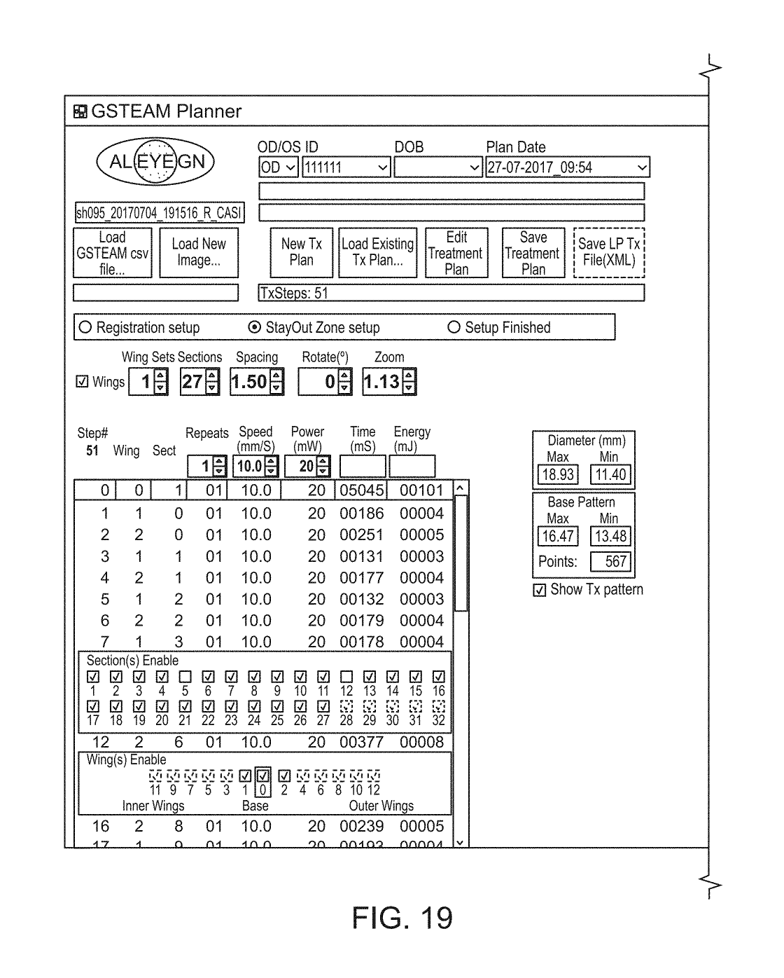

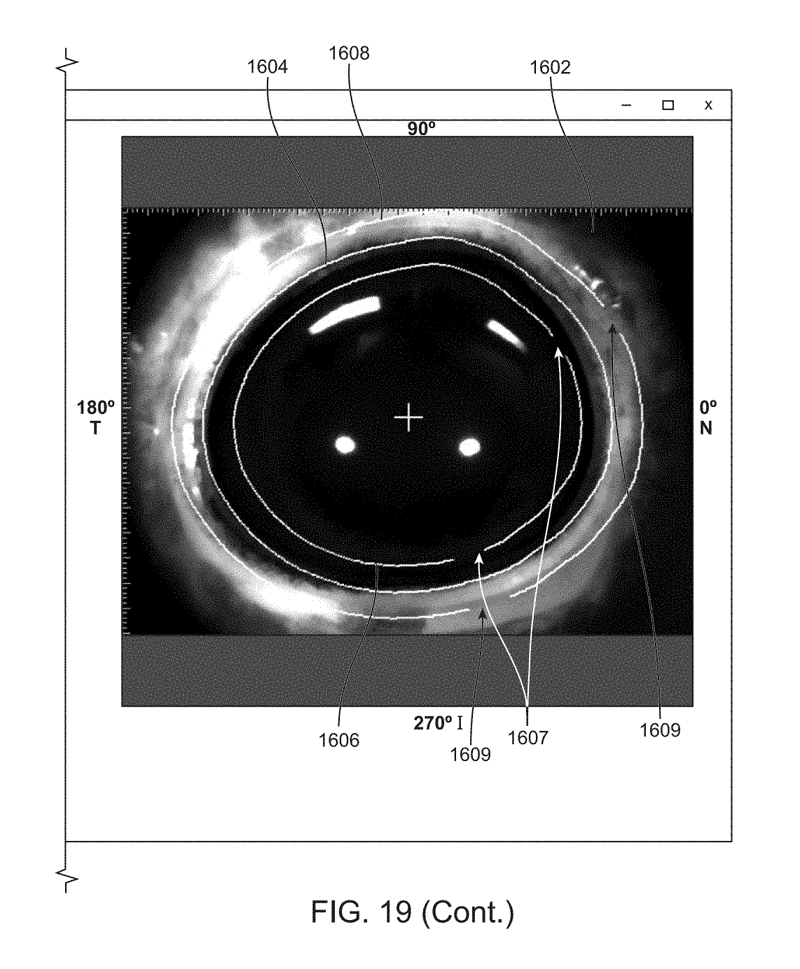

FIG. 19 shows a screen capture of the GUI 1600 on the stayout zone setup page with an anterior image of the eye 1602 comprising overlays of the limbus outline 1604, the inner treatment annulus 1606 with disabled section 1607, and the outer treatment annulus 1608 with disable section 1609. The spacing between the inner and outer treatment annuli 1606, 1608 was increased from 1 mm as shown in FIG. 18 to 1.5 mm by the user as described herein.

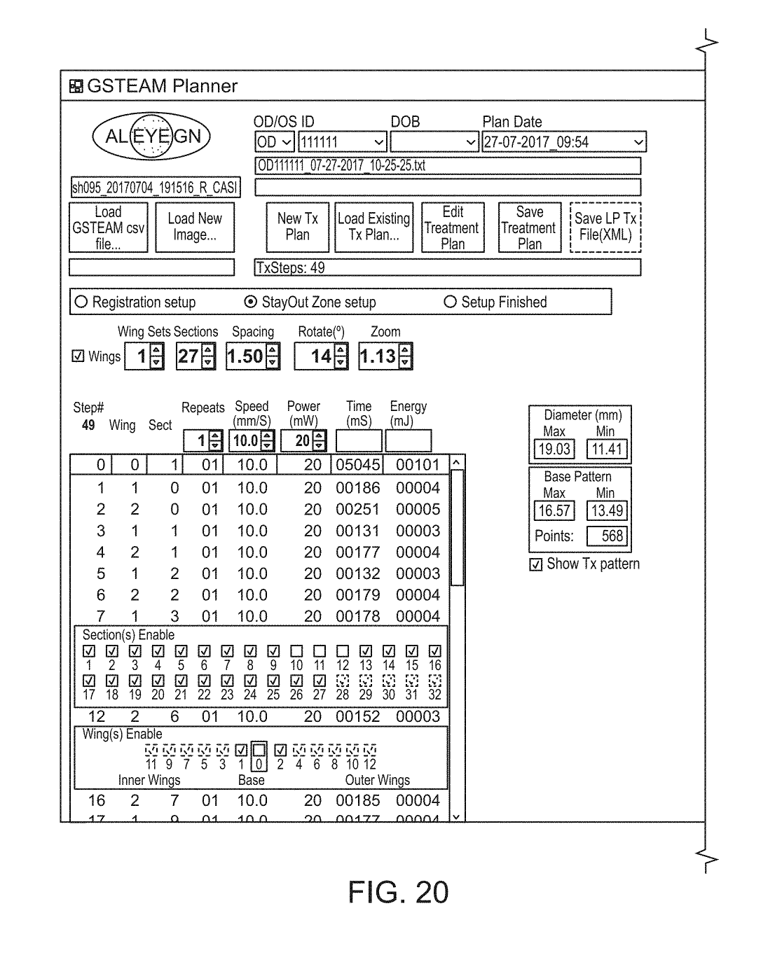

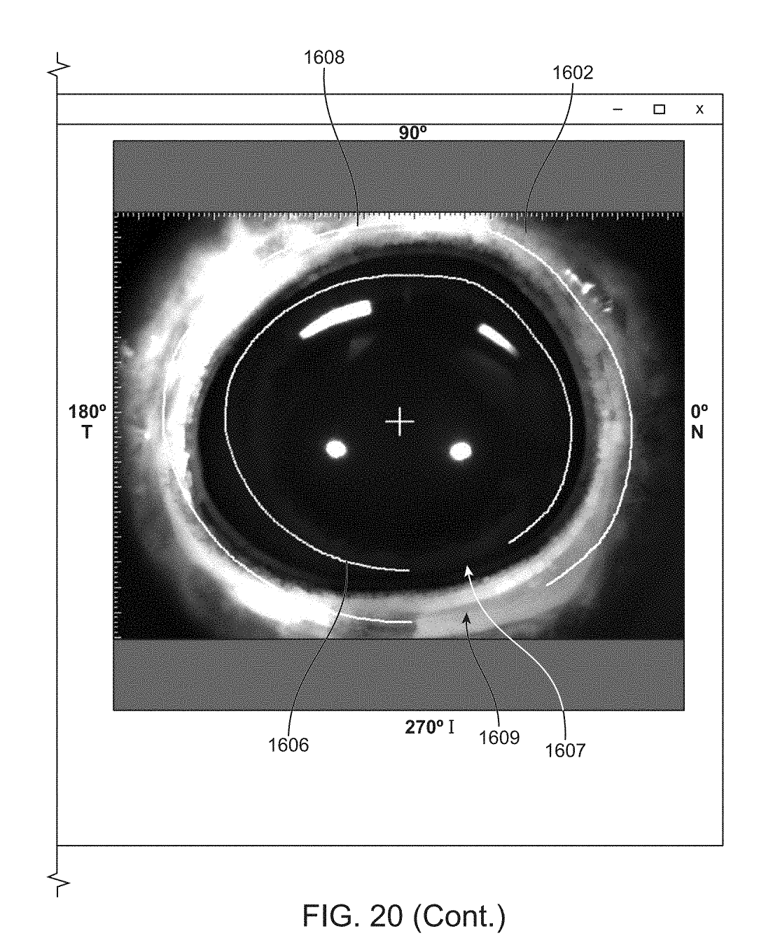

FIG. 20 shows a screen capture of the GUI 1600 on the stayout zone setup page with an anterior image of the eye 1602 comprising overlays the inner treatment annulus 1606 with disabled section 1607 and the outer treatment annulus 1608 with disabled section 1609. The treatment annuli 1606, 1608 have been rotated as described herein. The limbus overlay 1604 has been removed from view and disabled sections 1607, 1609 have been widened by the user.

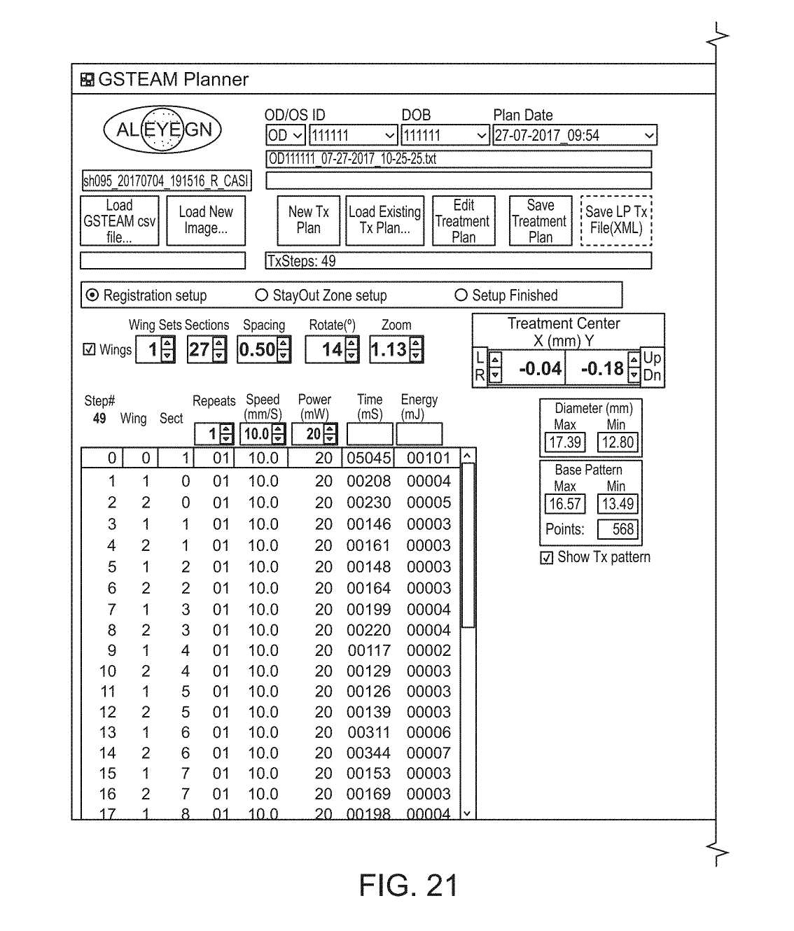

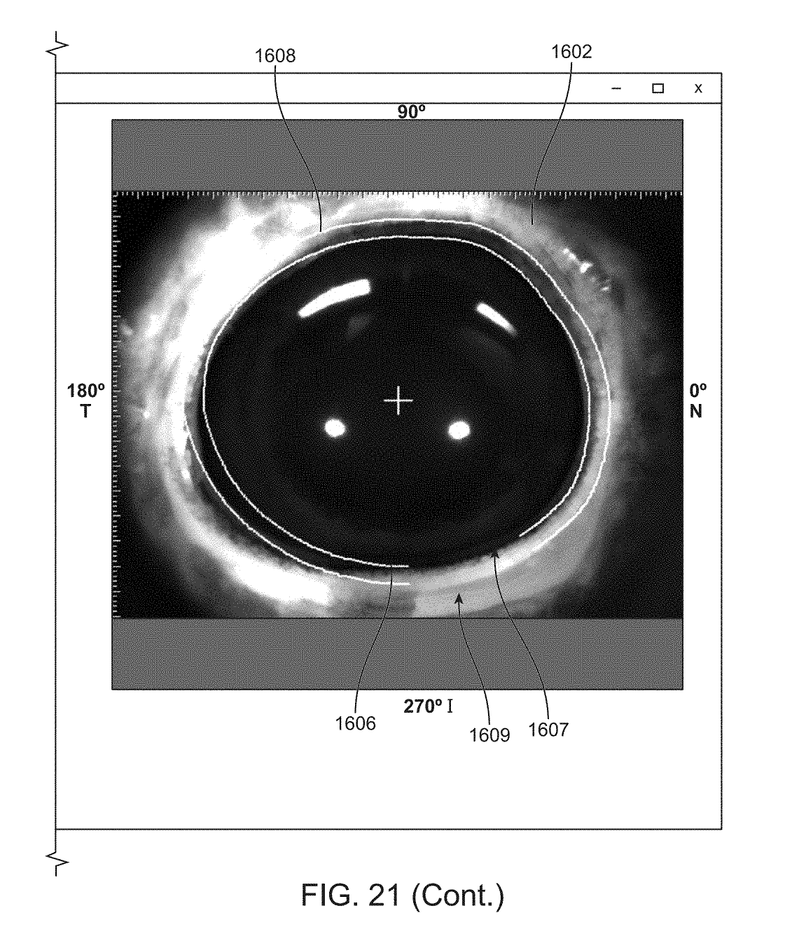

FIG. 21 shows a screen capture of the GUI 1600 on the registration setup page with the final treatment setup displayed on the anterior image of the eye 1604. Compared to the initially generated treatment annuli 1606, 1608 shown in FIG. 17, the final annuli 1606, 1608 have disabled sections 1607, 1609, have decreased spacing, are rotated, and have been shifted off-center.

The GUI 1600 may be configured to allow the user to adjust the treatment parameters and provide instructions to the processor. The GUI 1600 may be configured to allow the user to plan the treatment (e.g. set up the treatment pattern, parameters, etc.) prior to treatment. The GUI 1600 may display an anterior image of the eye 1602 to the user prior to, during, or after treatment as described herein. For example, as shown in FIG. 16, the anterior image of the eye 1602 may be displayed with an overlaid outline of the limbus 1604 generated as described herein. In some instances, it may also be desirable to display the treatment location 1606, 1608 on the anterior image of the eye 1602 as shown in FIGS. 17-21. Such overlays 1604, 1606, 1608 may be configured to remain on the real-time anterior image of the eye 1602 displayed during treatment so that the user may monitor treatment progress and accuracy. In some instances, it may be desirable to remove the overlays 1604, 1606, 1608 when treatment begins such that treatment may be monitored without being obscured by the overlay. The anterior image of the eye 1602 may for example be a video of the eye. The anterior image of the eye 1602 may for example comprise a static image taken by a camera. The anterior image of the eye 1602 may for example be a three-dimensional image of the eye.

In use, as an example, a user may upload an anterior image of the eye 1602 to the GUI 1600 using the load new image button 1615 or start the camera to generate an anterior image of the eye 1602 (e.g. a real-time video stream or static two dimensional or three dimensional image of the eye of a patient coupled to the system with a patient interface as described herein). The GUI may be set up with three “pages” as shown to reduce the number of fields visible to the user and reduce clutter on the display, for example to improve workflow efficiency. The pages may for example include a registration setup page which may be selected using the registration setup page button 1621, a stayout zone setup page which may be selected using the stayout zone setup page button 1623, and a setup finished page which may be selected using the setup finished button 1625. The user may then use the GUI to generate a treatment pattern based on the loaded image of the docked patient eye.

The anterior image of the eye 1502 may be loaded with a fiducial marker like the limbus outline 1604 described already drawn on it. Alternatively or in combination, the limbus outline 1604 may be loaded separately of the anterior image of the eye 1602 using the load GSTEAM csv file button 1614. The x-y coordinates used to generate the limbus outline 1604 may be generated from the anterior image of the eye 1602 using the process described in FIG. 8 for example. The name 1690 of the file which contains the limbus outline 1604 may be displayed to the user by the GUI 1600. Alternatively or in combination, the user may not upload a limbus outline 1604 and may manually pattern the treatment based on a visual inspection of the location of the limbus. Additional information about the treatment plan may be displayed by the GUI including which eye is being treated in the OD/OS drop down menu 1694, the patient identifier (“ID”) in the ID drop down menu 1695, the patient date of birth (“DOB”) in the DOB drop down menu 1696, and/or the date of the uploaded image/treatment plan in the plan date drop down menu 1691.

After the image 1602 has been displayed with the limbus outline 1604 (if desired), the user may then begin planning the treatment on the registration setup page. Previously generated treatment steps may be uploaded using the load existing Tx plan button 1618 or a new treatment plan may be set up using the new Tx plan button 1617. Treatment plans generated for future treatments may be saved using the save treatment plan button 1620. A previously generated treatment plan may be edited using the edit treatment plan button. While a juxtacanalicular treatment pattern is shown in FIGS. 17-21, it will be understood by one of ordinary skill in the art that any of the treatment patterns and parameters described herein may be planned for using the GUI.

In the case of juxtacanalicular treatment, a new treatment plan may include at least one limbus-shaped inner annulus 1606 positioned radially inward of the limbus outline 1604 and at least one limbus-shaped outer annulus 1608 positioned radially outward of the limbus outline 1604. The user may press the show Tx pattern button 1605 to display/remove the treatment annuli 1606, 1608 on the anterior image of the eye 1602 as desired. The annuli 1506, 1508 may be generated using the wings button 1631 and wing sets input field 1630. The button 1630 may be used to apply the wings (i.e. a pair of inner and outer annuli) 1606, 1608 while the input field 1631 may be used to adjust the number of wing pairs/sets 1606, 1608. While only a single inner annulus 1606 and a single outer annulus 1608 are shown, it will be understood by one of ordinary skill in the art that additional annuli may be added to the treatment plan as described herein by increasing the number of wing sets in the GUI.

The spacing/step size between annuli (in mm) may be adjusted using the spacing input field 1634. When no reference fiducial like the limbus outline 1604 is used, or when treatment is patterned without reference to the limbus, the wings button 1531 (or another button not shown) may be used to pattern treatment annuli on the eye, for example at a selected distance from the center of the eye.

The rotation of the treatment annuli may be adjusted using the rotate input field 1640. The angle of rotation may be configured to be consistent with conventional reference angles used by a physician when looking at the eye through an operating microscope, as will be understood to one of ordinary skill in the art. The temporal (“T”) direction may be at 0°, the nasal (“N”) direction may be 180°, the superior (“S”) direction may be at 90°, and the inferior (“I”) direction may be at 270° (shown here oriented towards the bottom of the display).

The center of the treatment pattern may be adjusted by changing the X and Y coordinates in the treatment center (X,Y) mm input field 1624.

The size of the treatment annuli 1506, 1508 may be adjusted using the zoom input field 1638.

The diameter 1635 of the entire treatment pattern may be displayed by the GUI. The diameter 1635 may be displayed as a minimum dimension across “min” and a maximum dimension across “max” the entire treatment pattern.

The diameter 1637 of the base pattern (i.e. the pattern upon which the treatment annuli are based such as the limbus outline 1604) may be displayed by the GUI. The diameter 1637 may be displayed as a minimum dimension across “min” and a maximum dimension across “max” the entire base pattern.

The treatment annuli 1606, 1608 may be made up of one or more treatment sections as described herein. The number of sections/arcs may be selected by the user using the sections input field 1632.

The user may select the number of times for each annulus to be treated (e.g. the number of full revolutions or repetitions along the annulus) using the repeats input field 1642.

The user may adjust the speed of the laser using the speed mm/sec input field 1648. The user may adjust the power of the laser using the power (mW) input field 1650.

Once the displayed treatment pattern and treatment parameters have been planned by the user, the user may press the new treatment plan button 1617 to display the calculated treatment steps in the treatment step menu 1610. The number of steps 1646 may be displayed. Additional steps may be added by generating new patterns/parameters and adding the next set of treatment steps to the menu 1610.

The treatment step menu 1610 may display a variety of information about each step of treatment based on the treatment parameters and patterns selected by the user during treatment planning. For example, information about which wing and which arc on said wing is being treated may be displayed in columns in the menu 1610. The user-selected repetition number 1642, the selected scan speed 1648, and the selected laser power 1650 may also be displayed in columns. Additional information may be calculated based on the treatment planned. Such information may be calculated/generated once the treatment steps are added to the menu 1610. Calculated and displayed information may include an estimate of the time to complete each step (in ms) 1656 and/or the amount of energy (in mJ) 1658 delivered in each step.

After determining the location of the treatment annuli 1606, 1608, the user may then push the stayout zone setup button 1623 to switch the page view to the stayout zone setup page (shown in FIGS. 18-20). The stayout zone setup page may include a section(s) enable selection area 1633 and a wing(s) enable selection area 1629 in the menu 1610.

The section(s) enable selection area 1633 may display a plurality of radio/option buttons, one for each section, which may enable treatment of the selected sections (shown as checked boxes) and disable treatment of the unselected sections (shown as unchecked boxes). Disabled sections 1607, 1609 may appear on the treatment pattern overlays 1606, 1608 as blank spaces along the annuli. FIGS. 18 and 19 show an exemplary treatment pattern with sections 5 (corresponding to disabled section 1607 on inner annulus 1606) and 12 (corresponding to disabled section 1609 on outer annulus 1608) unchecked and disabled. FIG. 20 shows an exemplary treatment pattern with sections 10-12 disabled. Sections may be enabled or disabled by the user depending on the desired treatment pattern, for example to avoid critical features of the eye, as will be understood by one of ordinary skill in the art.

The wing(s) enable selection area 1629 may display a plurality of radio/option buttons, one for each annulus, which may enable treatment of the selected annuli (shown as checked boxes) and disable treatment of the unselected annuli (shown as unchecked boxes). Disabled annuli may appear on the treatment pattern overlays as blank spaces between enabled annuli. FIG. 20 shows an exemplary treatment pattern with wing 0 (corresponding to limbus outline 1604) unchecked and disabled. Wings may be enabled or disabled by the user depending on the desired treatment pattern as will be understood by one of ordinary skill in the art.

Once the treatment pattern has been planned, the user may select the setup finished button 1625 to begin treatment.

Not all of the elements in FIGS. 16-21 are labeled in order to make the illustrations less cluttered and easier to see. While the GUI shown is configured to be used with a laser system, it will be understood by one of ordinary skill in the art that the GUI may be modified to be used with other energy sources or treatment systems as described herein.

In some instances, the treatment may be planned using the GUI(s) described herein during the therapeutic appointment, for example immediately prior to treatment. In some instances, the treatment may be planned prior to the therapeutic appointment. For example an image taken during a consultation prior to the therapeutic appointment may be used to plan the treatment any time before the patient returns for the therapeutic appointment. In some instances, the treatment may be planned prior to the therapeutic appointment and then adjusted during the therapeutic appointment prior to (or during) treatment. The GUIs described herein may for example be used to plan treatment using a static anterior image of the eye taken during a consultation appointment. During the therapeutic appointment, a video camera may be used to allow the physician user to align the pre-planned treatment pattern onto the eye docked into the patient interface of the system and make any adjustments necessary to set up the treatment as desired.

The anterior image of the eye may be calibrated or registered to the laser delivery system (e.g. the laser scanner) and/or to the imaging system (e.g. the OCT system) such that the treatment planned based on the anterior image of the eye fairly represents the treatment delivered to the eye during the STEM glaucoma treatment procedure.

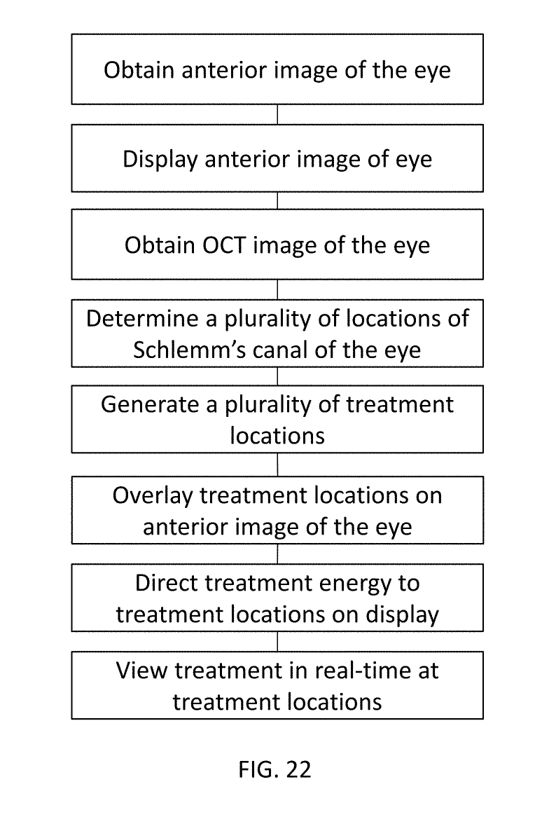

FIG. 22 shows a method for determining a target treatment location. The method may use one or more of the systems described herein. In a first step, an anterior image of the eye may be obtained by a camera or video recorder. In a second step, the image of the eye may be displayed to a user as described herein. In a third step, one or more OCT images of the eye may optionally be obtained. In a fourth step, a plurality of locations of Schlemm's canal (or the limbus or other fiducial described herein) may be determined from the anterior image of the eye, the one or more OCT images of the eye, or any combination thereof. The plurality of locations of Schlemm's canal may be estimated manually by the user or automatically by the processor. The plurality of Schlemm's canal locations may optionally be registered with a corresponding plurality of anterior image locations. In a fifth step, a plurality of treatment locations for the eye may be determined in response to the plurality of locations of Schlemm's canal. The plurality of treatment locations may be determined manually by the user or automatically by the processor. In a sixth step, the treatment locations may be overlaid onto the anterior image shown on the display. The treatment locations may optionally be adjusted or approved by the user. In a seventh step, treatment energy may be directed to the treatment locations displayed on the image by an energy source and scanner as described herein. In an eighth step, the treatment may be viewed in real-time at the treatment locations in order to adjust or halt treatment if movement of the eye occurs.

In some instances, a processor may be provided. The processor may be configured with instructions for perform a series of steps illustrated in FIG. 22. In some instances, the processor may provide instructions to obtain an anterior image of the eye. For example, the anterior image of the eye may be obtained with a camera with aid of the processor. In some instances, the processor may be configured with instructions for receiving an anterior image of the eye.

In some instances, the processor may provide instructions to display the anterior image of the eye. In some instances, the processor may provide instructions to obtain OCT image(s) of the eye. In some instances, the processor may provide instructions to determine a plurality of locations of Schlemm's canal of the eye. The processor may estimate in some instances the plurality of Schlemm's canal locations in response to the anterior image of the eye. Alternatively or in addition, the processor may estimate the plurality of Schlemm's canal locations in response to the plurality of OCT images of the eye.

In some instances, the processor may be configured with instructions to generate a plurality of treatment locations. Optionally, the processor may be configured with instructions to generate the plurality of treatment locations for the eye in response to the plurality of Schlemm's canal locations.

In some instances, the processor may provide instructions to overlay treatment locations on the anterior image of the eye. The processor may be configured with instructions to overlay the plurality of treatment locations and the plurality of Schlemm's canal locations on the anterior image of the eye. Optionally, the processor may be further configured to register the plurality of locations of Schlemm's canal with a corresponding plurality of anterior image locations.

In some instances, the processor may provide instructions to direct treatment energy to treatment locations on the display. In some instances, the processor may be configured with instructions to alternate treatment at a first plurality of treatment locations with treatment at a second plurality of treatment locations as described herein. Optionally, the processor may be configured with instructions to generate a third plurality of treatment locations located radially outward from the second plurality of treatment locations to generate vacuoles or increase a size of vacuoles in a sclera of the eye.

Optionally, the processor may be configured with instructions to generate a treatment table. The treatment table may comprise a plurality of coordinate reference locations corresponding to the plurality of treatment locations overlaid on the anterior image. Optionally, the energy source directed to the eye may comprise a pulsed energy source wherein each of the plurality of coordinate references corresponds to a pulse from an energy source.

In some instances, the processor may provide instructions to display treatment in real-time at the treatment locations.

Although the steps described above show a method of acquiring an image of an eye and treating the tissue at a treatment region selected by a user, one of ordinary skill in the art will recognize many variations based on the teachings described herein. The steps may be completed in a different order. Steps may be added or deleted. Some of the steps may comprise sub-steps. Many of the steps may be repeated as often as necessary to treat the tissue as desired.

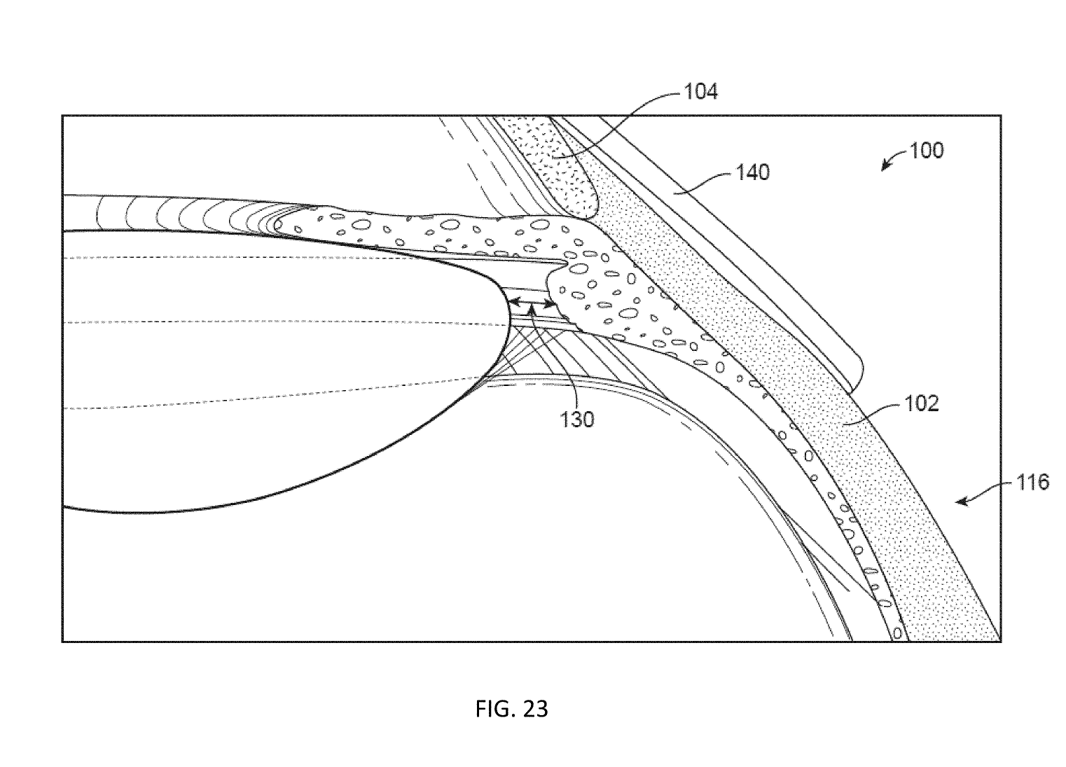

FIG. 23 illustrates a heat sink 140 placed over the eye 100 of FIG. 2 in order to treat glaucoma, in accordance with embodiments. The heat sink 140, for example a chilled contact lens, can be inserted over an outer portion of the eye 100 including the cornea 104, sclera 102, and conjunctiva 116, in order to conduct heat away from the outer portion of the eye 100 during the treatment procedure. The heat sink can be made of any suitable material. For example, the heat sink can include a material transmissive to wavelengths of light energy (e.g., sapphire or diamond-like carbon transmissive to certain wavelengths of IR light), so that the eye tissue beneath the heat sink can be heated with absorbed light energy.

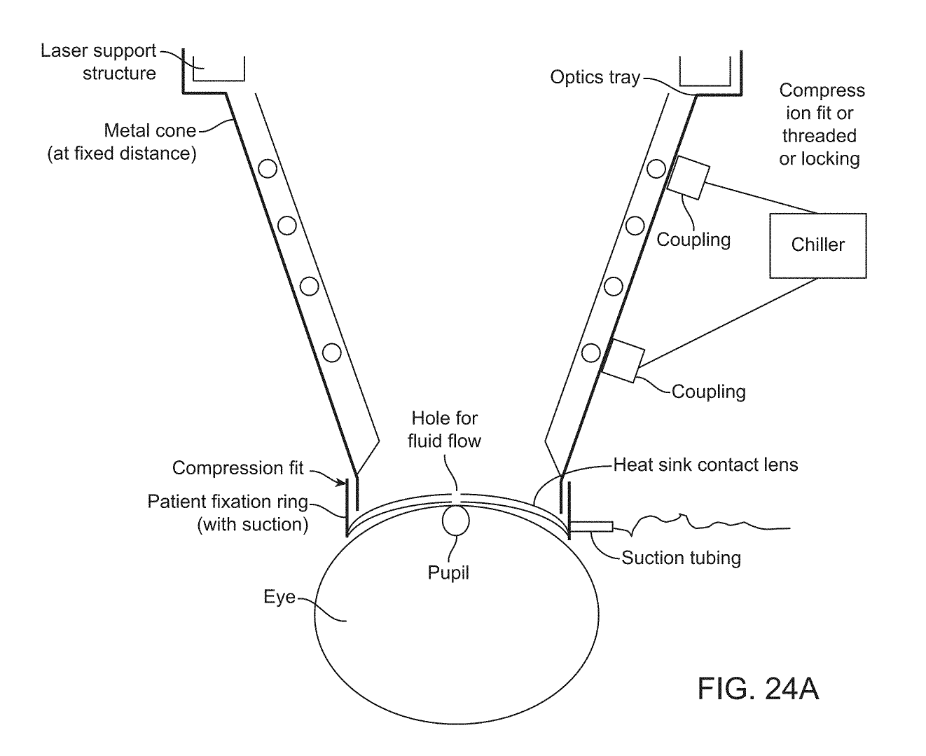

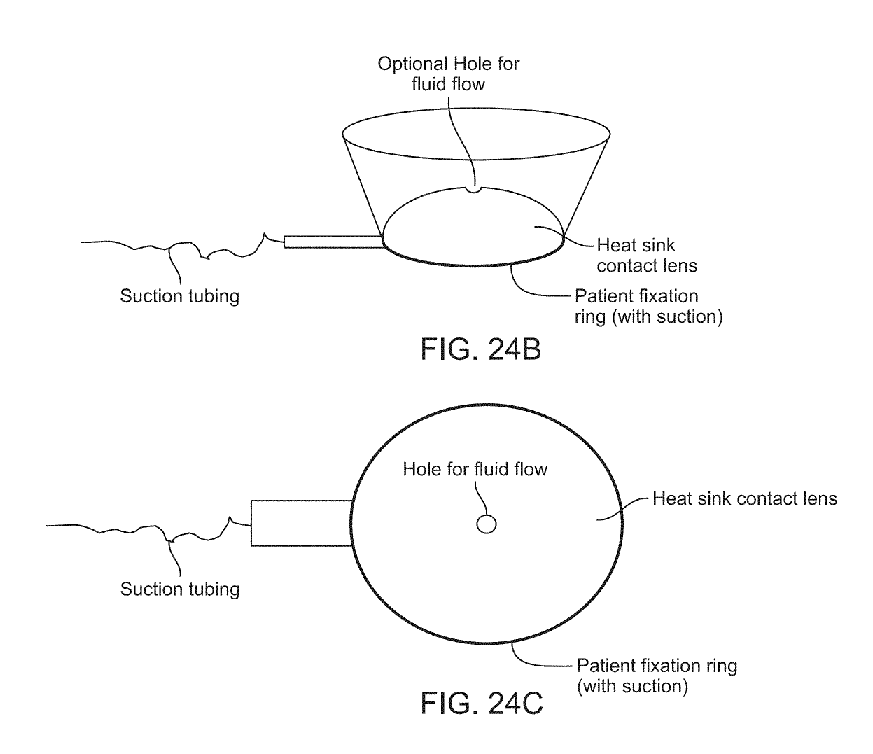

FIGS. 24A-24C show a structure for coupling an energy source to a surface of an eye. FIG. 24A shows a side view of a structure for coupling an energy source to a surface of an eye. FIG. 24B shows a side view of a structure for coupling an energy source to a surface of an eye. FIG. 24C shows a top view of a structure for coupling an energy source to a surface of an eye.

The structure may comprise a cone structure. The cone may be configured to remove heat from a surface of an eye. The cone may be composed of a material having a high thermal conductivity. The cone may be a metal cone.

The cone may be coupled to a laser support structure and an optics tray. The laser support structure may support one or more laser sources, as described herein. The optics tray may support one or more optical components that direct one or more lasers to a surface of an eye, as described herein.

The cone may be coupled to a patient fixation ring. The patient fixation ring may be configured to form an air-tight seal with a surface of the cone. The patient fixation ring may be coupled to a surface of the cone using a compression fitting. The patient fixation ring may be configured to provide suction. For instance, the patient fixation ring may be coupled to suction tubing. The suction may be provided by connecting a syringe to the suction tubing and withdrawing the syringe. The suction tubing may comprise a vacuum pressure sensor. The pressure sensor may be used to determine that the coupling structure is properly connected to an eye.

The patient fixation ring may be coupled to a heat sink contact lens. The heat sink contact lens may be seated within the patient fixation ring. The heat sink contact lens may be composed of a material having a high thermal conductivity. The heat sink contact lens may be composed of sapphire. The heat sink contact lens may be composed of diamond or a diamond-like material. The heat sink contact lens may comprise a hole located at approximately the center of the heat sink contact lens. The hole may allow for the flow of fluids (such as air) away from the eye. The heat sink contact lens may have an outer diameter of about 15 mm, 16 mm, 17 mm, 18 mm, 19 mm, or 20 mm. The heat sink contact lens may have an outer diameter of less than 15 mm. The heat sink contact lens may have an outer diameter of greater than 20 mm. The heat sink contact lens may have a thickness of about 0.5 mm, 1 mm, or 1.5 mm. The heat sink contact lens may have a thickness of less than 0.5 mm. The heat sink contact lens may have a thickness of greater than 1.5 mm.

The cone may be positioned on a counter-weighted moveable arm such that no weight rests on the eye when the cone is docked to the patient fixation ring. The cone may have a fixed working distance such that the distance between the surface of the eye and the energy source may be constant between patients. The cone may be thermally controlled, for example with a fluid-based (such as water-based) heat exchanger or Peltier cooler, in order to help maintain the desired temperature of the patient interface and/or contact lens. Controlling the temperature of the cone may allow the preservation of tissues within the eye during an interaction with an energy source. For instance, cooling the cone may allow for the preservation of the epithelium during heating with a laser source.

In some embodiments, the cone may be thermally controlled using a fluid-based heat exchanger. The cone may comprise one or more fluid channels. The fluid channels may be fluidically coupled to a chiller through one or more couplings and one or more tubes or hoses. The couplings may be threaded couplings. The couplings may be compression couplings. The chiller may circulate a cooling fluid (such as water, ethylene glycol, or another liquid coolant) through the fluid channels in order to cool the cone. The chiller may cool the cone to a temperature less than 37° C., less than 30° C., less than 25° C., less than 20° C., less than 15° C., less than 10° C., less than 5° C., or less than 0° C. The chiller may comprise a water exchanger. The water exchanger may have a lateral footprint of approximately 8 inches×8 inches. The water exchanger may draw an electric power of approximately 160 W.

In some embodiments, the cone may be thermally controlled using a thermoelectric cooler. The thermoelectric cooler may comprise a Peltier cooler. The Peltier cooler may be placed in thermal connection with the heat sink lens. The Peltier cooler may be located on the counter-weighted moveable arms. The Peltier cooler may cool the cone to a temperature less than 37° C., less than 30° C., less than 25° C., less than 20° C., less than 15° C., less than 10° C., less than 5° C., or less than 0° C.

In some embodiments, the thermoelectric cooler may be a disc-shaped thermoelectric cooler located on the top of the patient interface cone.

The system may be operated by optionally applying vacuum to the eye, aiming an illumination beam at the eye, and obtaining an OCT image of the eye. The OCT image may provide a baseline image of the eye prior to treatment. A treatment may be started once the heat sink contact lens has been secured in place by the vacuum. An OCT image may be obtained following treatment. The OCT image may be compared with the baseline image to obtain a precise measurement of changes induced by the treatment.

In some instances, medicaments may be added to the eye prior to and/or after treatment to further protect the corneal surface against thermal insult and/or stabilize treatment effects. Eye drops may contain medicaments which sequester and/or protect against collagen degradation and may be applied to the eye prior to and/or after treatment. In some instances, the medicaments may be collagen-sparing. The eye drops may contain medicaments such as hyaluronate or the like, polymers such as hydroxypropyl methylcellulose, and/or dissacharides optionally selected from the group consisting of Sucrose (table sugar, cane sugar, beet sugar, or saccharose), Lactulose, Lactose (milk sugar), Maltose (malt sugar), Trehalose, Cellobiose, Chitobiose, Kojibiose, Nigerose, Isomaltose, Trehalose (for example β,β-Trehalose or α,β-Trehalose), Sophorose, Laminaribiose, Gentiobiose, Turanose, Maltulose, Palatinose, Gentiobiulose, Mannobiose, Melibiose, Melibiulose, Rutinose, Rutinulose, Xylobiose, and any combination thereof.

In some instances, a topical anesthetic may be applied to the eye prior to or after treatment. Such anesthetics may include anesthetics with a tropane skeleton optionally selected from the group consisting of the Amylocaine, Articaine, Benzocaine, Bupivacaine, Butacaine, Carticaine, Chloroprocaine, Cinchocaine/Dibucaine, Cyclomethycaine, Etidocaine, Eucaine, Fomocaine[55], Fotocaine[55], Hexylcaine, Levobupivacaine, Lidocaine/Lignocaine, Mepivacaine, Meprylcaine/Oracaine, Metabutoxycaine, Phenacaine/Holocaine, Piperocaine, Pramocaine/Pramoxine, Prilocaine, Propoxycaine/Ravocaine, Procaine/Novocaine, Proparacaine/Alcaine, Quinisocaine, Risocaine, Ropivacaine, Tetracaine/Amethocaine, Trimecaine, and any combination thereof.

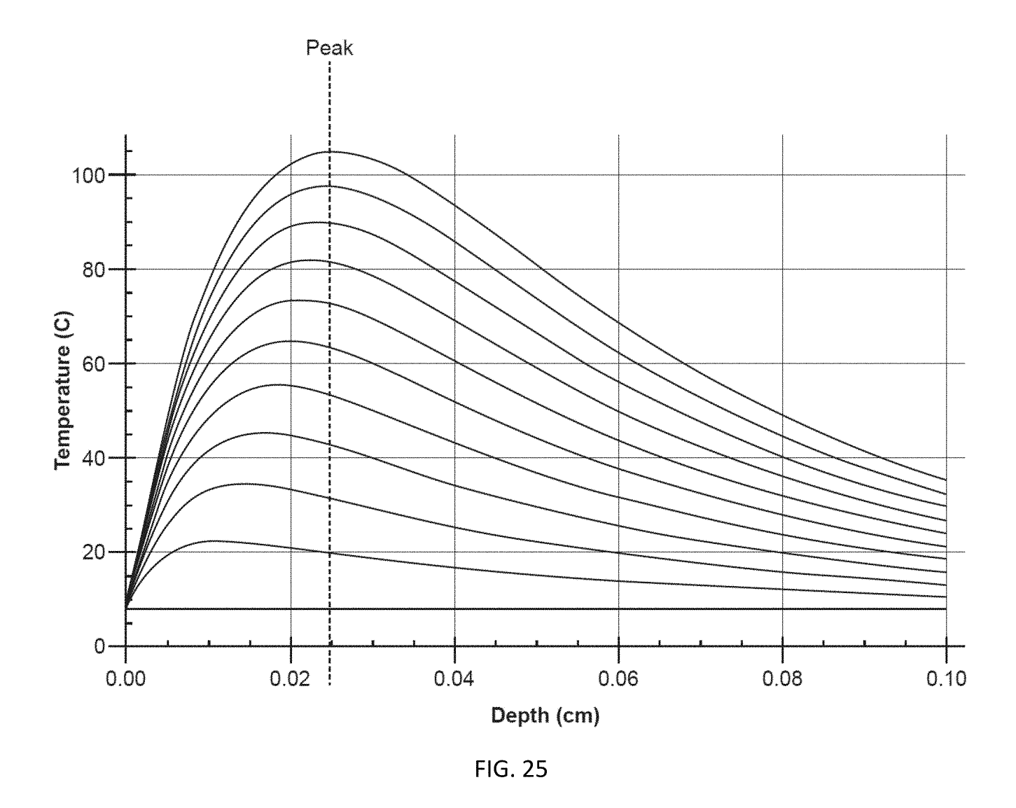

FIG. 25 shows temperature profiles of an eye treated with a laser beam with the eye coupled to a chilled lens based on computer modeling. FIG. 25 shows a plot of a finite element analysis model of temperature (in degrees C.) as a function of scleral tissue depth (in mm). The modeling was performed with commercially available COMSOL finite element software to model heat transfer and tissue temperature with energy delivered to the sclera. Temperature profiles of an eye treated with a 2.1 um laser beam were simulated with the eye coupled to a chilled lens using computer modeling. The finite element analysis assumed anisotropy in the radial and axial dimensions. Each line represents a different amount of energy deposited within the tissue. Changes in the simulated power and/or time were used to alter the total simulated energy deposited within the tissue. For each of the treatment parameters simulated, the tissue reached a peak temperature at about 250 um deep. The simulation was conducted assuming a laser intensity of 200 W cm⁻² and tissue absorptivity at 2.1 um of 20 mm⁻¹. The total amount of energy deposited and peak temperature is related to the treatment time. These plots show increased peak temperatures and deeper locations of the peak temperature with increased treatment times.

The chilled lens was modeled at the surface with a fixed temperature of about 6 to 8 C, assuming that the lens would remain at the substantially the same temperature to which it was cooled prior to or during (via active cooling methods) treatment. Such temperatures may for example be reached by placing a cooled lens on the surface of the eye for a predetermined amount of time, for example about 10 to about 30 seconds, prior to initiating treatment as described herein. The amount of time sufficient to cool and protect the surface of the eye may vary depending on the thickness of the cornea and the extent of suction applied by the system to maintain contact between the lens, patient interface, and the surface of the eye.

In some instances, a temperature of about 60 to about 70 degrees may be reached at a depth of about 50 um to about 400 um in the sclera. A temperature of about 60 to about 70 degrees may be reached at a depth of about 50 um to about 300 um in the cornea. The conjunctiva and/or the epithelium may remain substantially intact during heating of the underlying tissue due to the use of a cooling element or heat sink, such as a chilled contact lens, to inhibit damage to these sensitive surface tissues of the eye. The surface of the sclera may be chilled so as to remain at or near 4 C.

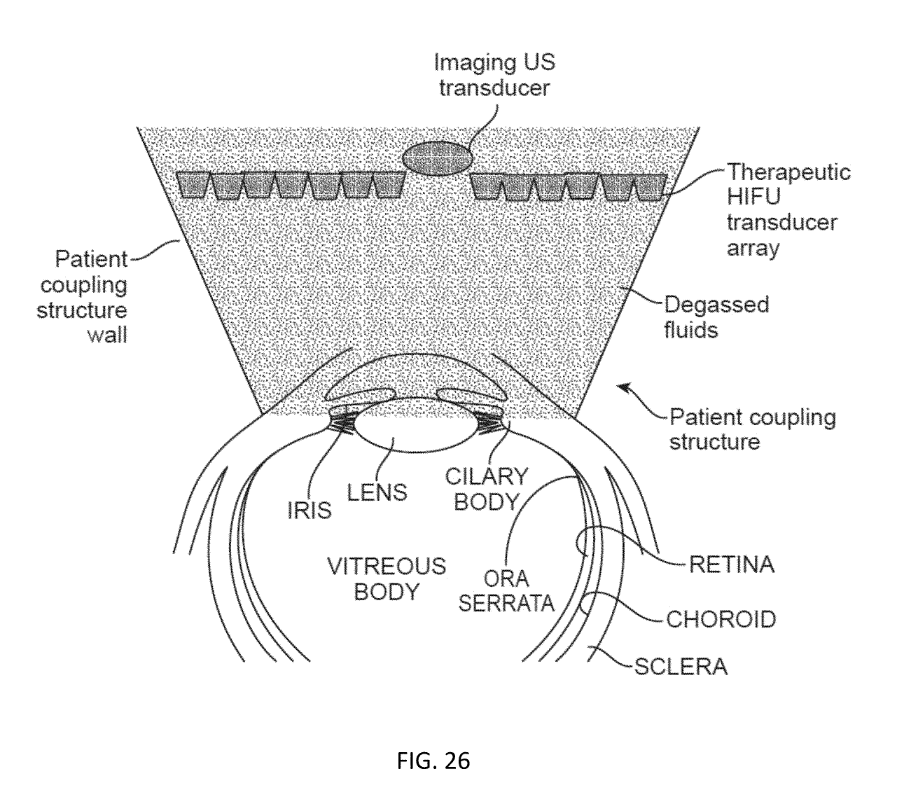

FIG. 26 shows a treatment setup for STEM adjustment using ultrasound, for example high intensity focused ultrasound (HIFU), as an energy source. In one embodiment, a therapeutic HIFU transducer array which comprises a centrally positioned imaging system, for example an ultrasound transducer or OCT fiber, may be coupled to the eye with a patient coupling structure comprising a conic-shaped wall and a degassed fluid therein. The fluid interface may serve as a space to tightly focus the ultrasound beam to the desired treatment zone. Alternatively or in combination, the fluid may allow for greater control and/or a greater range over depth from the tissue surface. The fluid may further be used to control the temperature of surface tissue during exposure to HIFU during treatment. The fluid is preferably chilled to about 4° C. and may be one or more of a gel, a gel pack, water or trehalose. The HIFU array may be aimed at a cavitational zone appropriate for the STEM adjustment treatment area.

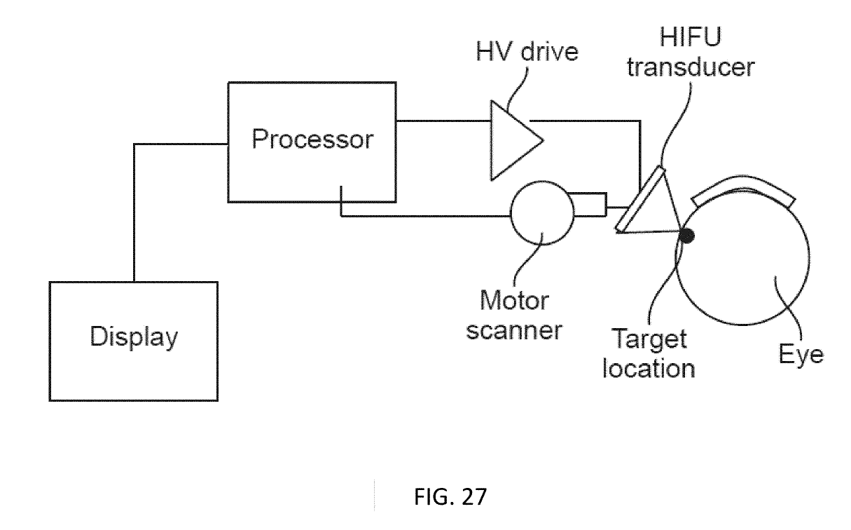

FIG. 27 shows a STEM adjustment system comprising HIFU. The system comprises a HIFU array focusing ultrasound energy to a location inside the eye. A motor scanner can optionally be coupled to the ultrasound array to direct the treatment energy to the target locations of the eye. A processor may be coupled to a high voltage drive (HV) to drive the array. The processor can be coupled to the motor scanner to move the array during treatment. A display can be coupled to the processor to show an image of the eye. The image of the eye can be generated with imaging frequencies and wavelengths and the HIFU can be delivered to the eye with HIFU wavelengths as described herein.

One or more of the motor scanner, HIFU transducer, patient coupling structure, and imaging system may comprise an energy delivery system. The energy may be directed by the ultrasound energy delivery system to the eye or a hand held probe.

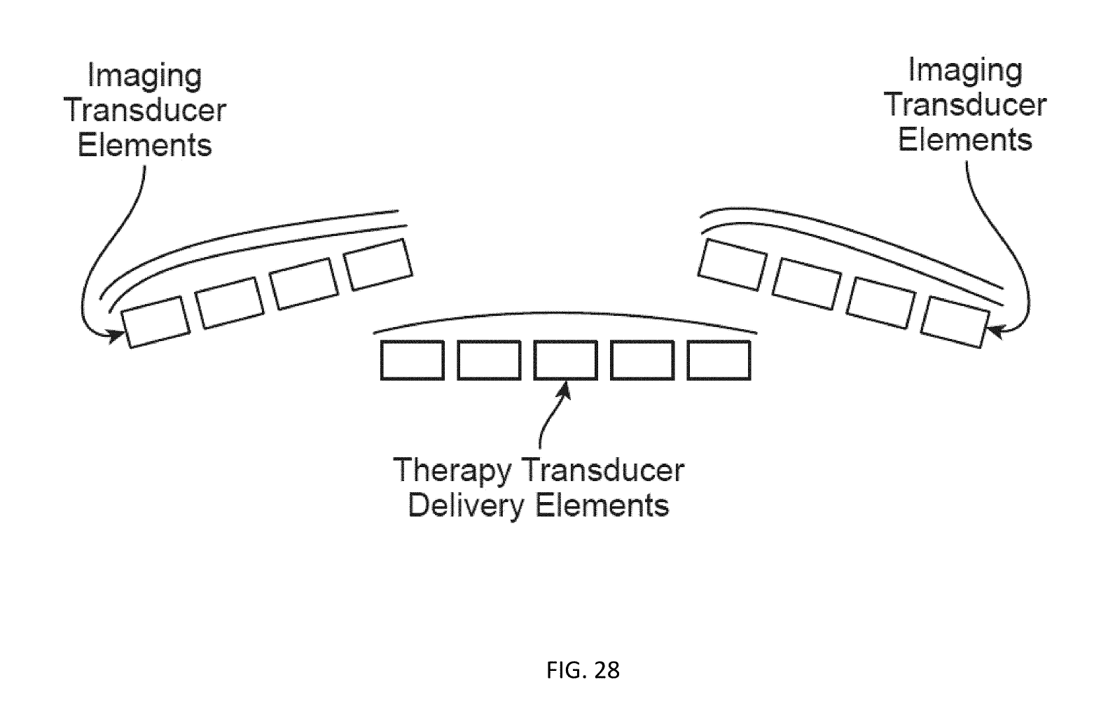

FIG. 28 shows a HIFU array coupled to an imaging apparatus. A pair of ultrasound imaging arrays and a HIFU array are arranged for real-time imaging during treatment. The imaging transducer elements and therapy transducer and elements can be coupled to the processor as disclosed herein. Coupling the imaging apparatus to the HIFU transducer allows for passive cavitation detection and imaging feedback to guide and inform treatment.

The HIFU transducer may comprise one or more of a phased array, a discrete array, an annular array, a spherical array, a spherical phased array, or any combination thereof. The HIFU transducer may be combined with an imaging apparatus, for example embedded OCT sensors. Additionally, the transducer may be fabricated to allow for opto-acoustic excitation for precise theranostic delivery.

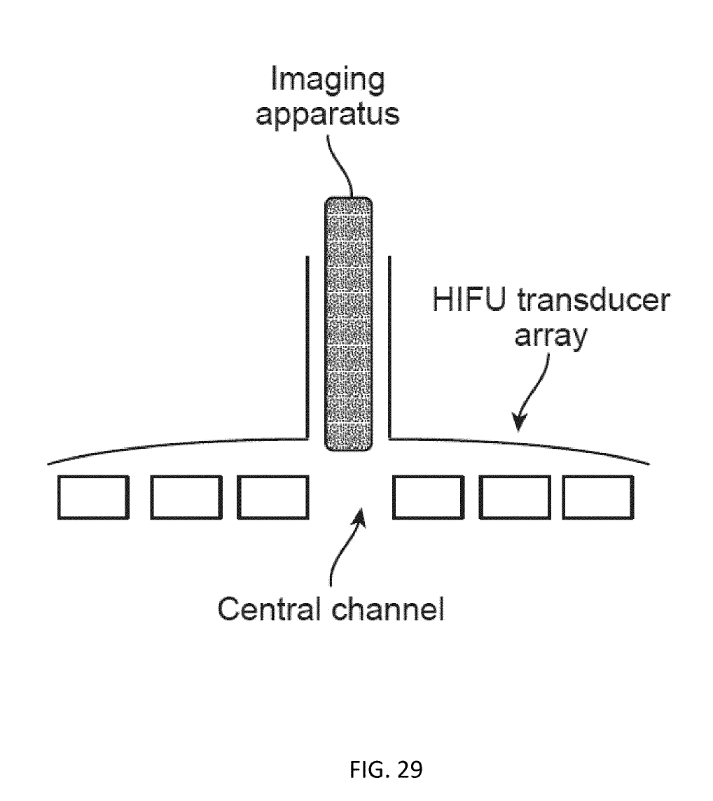

FIG. 29 shows another embodiment of a HIFU array coupled to an imaging apparatus. The HIFU array in this embodiment comprises a transducer with central channel in which the imaging apparatus may be disposed. For example, the imaging apparatus may be an OCT fiber optic cable. The OCT fiber may be disposed inside a channel extending from the center of the therapy transducer and can allow for real-time imaging of tissue at one or more times before, during, or after treatment with the HIFU array.

The HIFU array may be coupled to a number of imaging systems, including but not limited to MRI, UBM, ultrasound imaging, OCT, OCE, or US elastography.

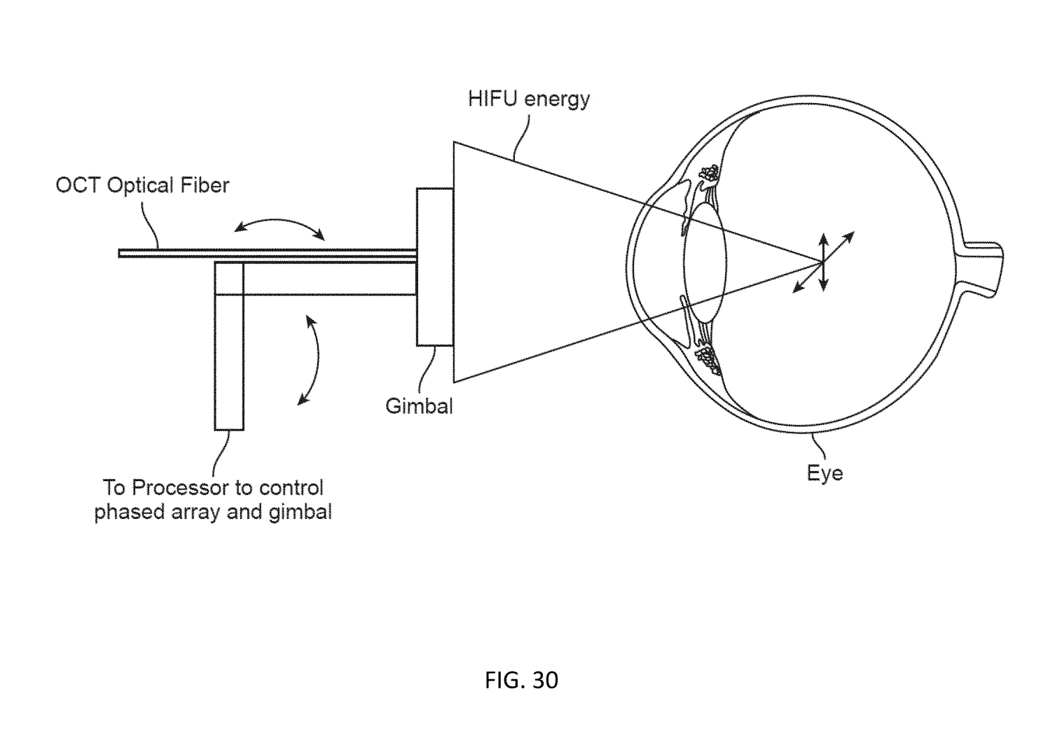

FIG. 30 shows a schematic of an embodiment of a one-dimensional high-intensity focused ultrasound (HIFU) system which may be used to perform the glaucoma methods described herein. In some instances, a laser or other energy source may be provided instead of or in addition to the HIFU system shown. The system may comprise a HIFU transducer array, for example a phased array, coupled to a gimbal for support and movement control. The HIFU array may for example be mounted to an end of the gimbal. The gimbal may provide three degrees of freedom for spot scanning. The gimbal and phased array may be coupled to a processor (not shown) which controls the movement of the gimbal, and thus the phased array and HIFU energy, so as to pattern the HIFU energy beam in a first direction. Alternatively or in combination, the phased array may steer the HIFU energy beam in a second direction transverse or at an angle to the first direction. Thus, the HIFU energy beam may be patterned for treatment using any of the treatment patterns described herein which occur in one or two-dimensions. The HIFU array may be coupled to an imaging system, for example an OCT optical fiber, so as to image the eye before, during, or after treatment in real-time as described herein.

Alternatively or in combination, the HIFU array or gimbal may be mounted to an x-y motorized translation stage which may move the transducer in x, y, or both x and y during treatment. The x-y motorized translation stage may be controlled by a computer or processor as described herein.

Alternatively or in combination, the phased array of the HIFU system may further be configured to provide treatment at depths (e.g. treatments in z) within the tissue. Alternatively or in combination, the HIFU transducer or gimbal may be mounted on an x-y-z translation stage in order to treat tissue at varying depths. The x-y-z translation stage may be under computer or processor control to allow for up to 3-D scanning. Alternatively or in combination, the HIFU transducer may be a 2-D phased array to allow for 3-D volumetric scanning of the tissue.

### EXPERIMENTAL

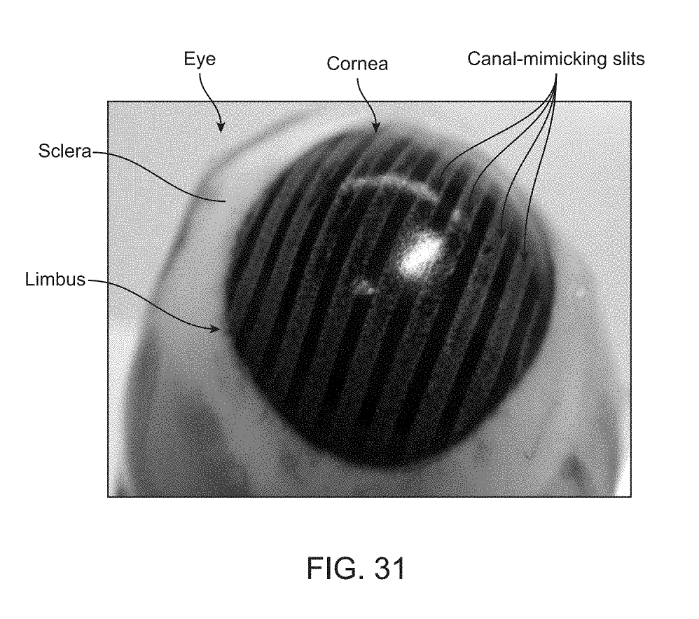

FIGS. 31-40B show results from proof of concept experiments done in ex vivo porcine eyes showing that the STEM systems and methods described herein may be used to widen (or dilate or stretch) Schlemm's canal, which may improve glaucoma.

FIG. 31 shows slits (or canal reference cuts) generated in ex vivo porcine corneas generated by a TissueSurgeon™ femto laser prior to treatment (“pre-op”) with the STEM laser system described herein. The slits were about 800 um to about 900 um wide and about 500 um deep parallel to the iris/cornea surface in order to mimic Schlemm's canal. The slits were injected with glass microbeads for increased contrast and visualization with OCT (via increased scattering) in order to monitor the effects of treatment (pre-op vs. post-op). Laser energy was delivered to the eye through a cooled tissue-sparing sapphire contact lens in order to shrink tissue of the cornea and/or sclera and induce axial strain on the canal-like slits to displace the slits and mimic opening of Schlemm's canal.

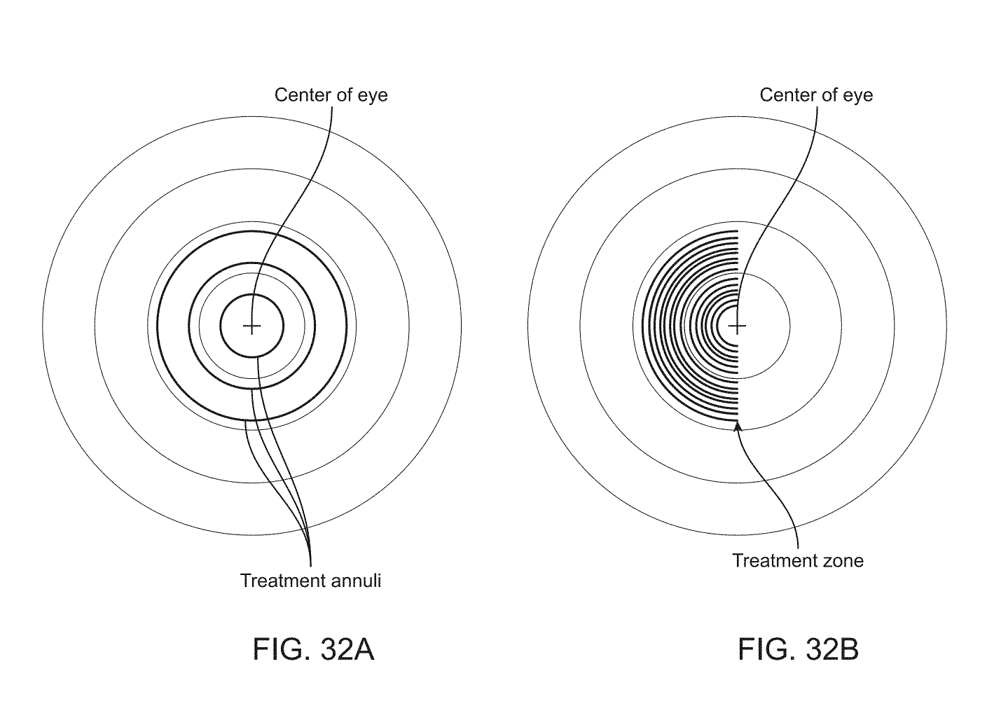

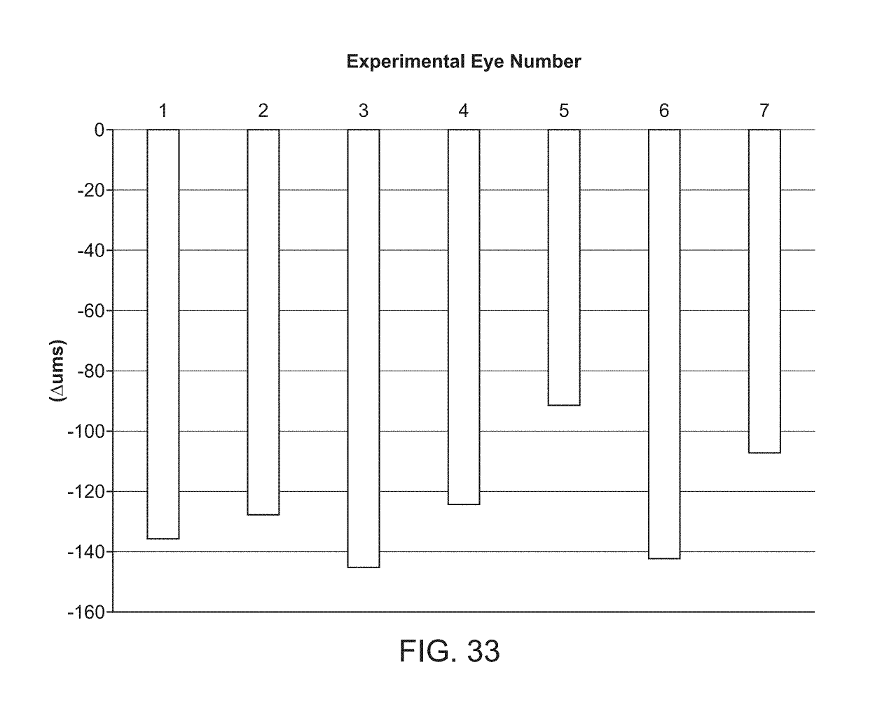

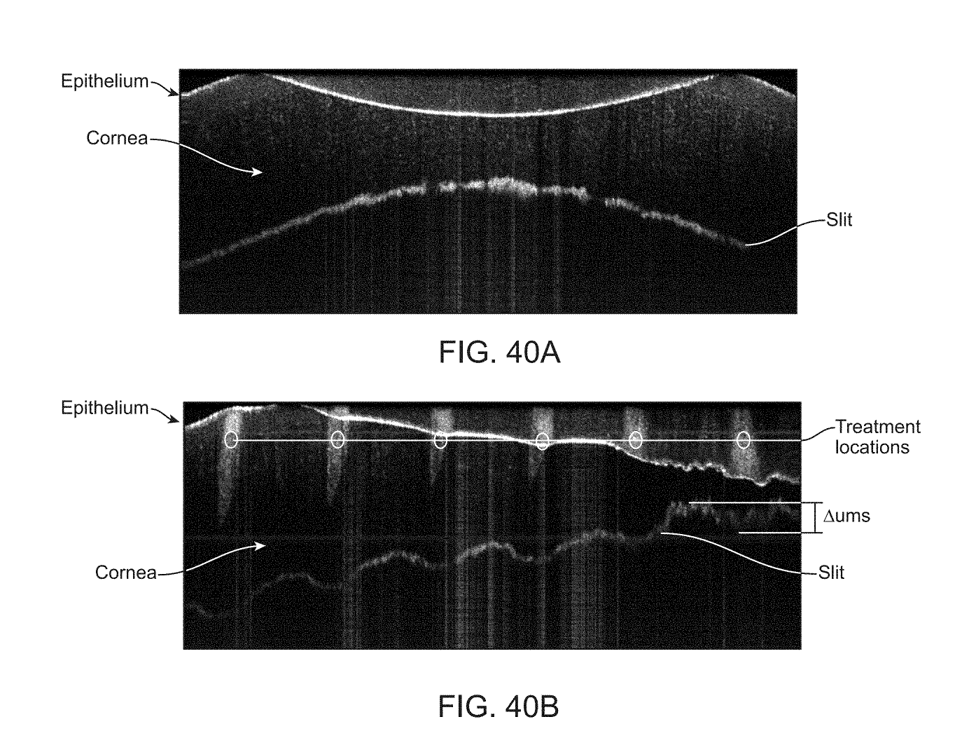

FIGS. 32A-32B show schematics of test patterns used to treat porcine corneas ex vivo to induce axial movement of femto-slits generated as described in FIG. 31. Various treatment parameters and patterns were used to induce translocation of the canal-mimicking slits. Parameters including wavelength, laser power, scan speed, and spot diameter as well as annular patterning and location of shrinkage energy deposition were varied in order to generate strain in the tissue. Generation of strain sufficient to reach a targeted minimum of 60 um peak to valley translocation of the slits (which equates to roughly half of the diameter of a typical glaucomatous Schlemm's canal) was repeatably achieved in porcine eyes (see FIGS. 33-40B) with a 2.1 um laser with a laser power of 300 mW, a scan speed of 25 mm/sec, and 200 um spot size. Energy was transmitted to the eye through a chilled sapphire contact lens in contact with the cornea in order to protect the epithelium and vasculature of the eye. FIG. 32A shows a pattern which was used to induce strain in the corneal slits. 360 degree annular shrinkage was induced in the cornea at 3 mm, 6 mm, and 9 mm radially outward from the center of the eye (i.e. optical axis of the eye). FIG. 32B shows another pattern which as used to induce strain in the canal-mimicking slits. 180 degree arcuate shrinkage was induced in the cornea between 2 mm and 9 mm radially outward from the center of the eye with 0.5 mm steps between treatment arcs. Other patterns, for example 360 degree annular shrinkage between 2 mm and 9 mm radially outward from the center of the eye with 1.4 mm steps between treatment annuli (see FIGS. 40A-40B), were also found to induce strain and translocation of the slits between the treatment annuli.

FIG. 33 shows the results of seven ex vivo porcine eyes with femto laser generated canal-mimicking slits in the cornea treated to induce corneal tissue shrinkage and anterior translocation of the slits between the treatment locations. The slits were used to model STEM treatment-induced anterior translocation of Schlemm's canal as described herein in order to determine how the glaucoma treatment systems and methods can be used to open Schlemm's canal. OCT images of the eye were taken prior to treatment (“pre-op”) and after treatment (“post-op”) and the STEM-induced patterned strain modulation at 500 um deep was measured as a change in microns (Δums) between pre-op peaks and post-op peaks induced in the slits in response to STEM-induced focal axial forces. Peak to peak changes were measured to be more than about 60 um±20 um in the post-op OCT images compared to the corresponding pre-op images. These peak to peak changes in the canal-mimicking slits suggest that similar treatment parameters and/or patterns may be useful in expanding Schlemm's canal, for example from a typical glaucomatous diameter of about 120 um to about 180 um or more.

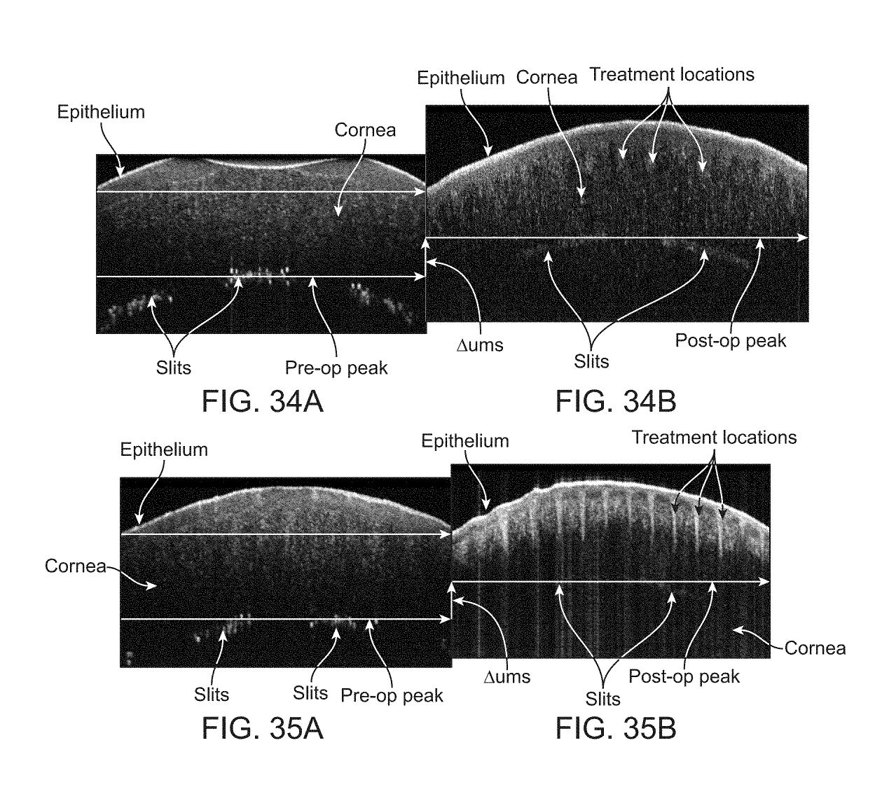

FIGS. 34A-41E show optical coherence tomography (“OCT”) images of eyes.

FIGS. 34A and 34B show OCT images of the pre-operative and post-operative locations, respectively, of femto-slits in an ex vivo porcine eye with STEM-induced translocation (Δums) from a pre-op peak location to a post-op peak location via 360 degree annular treatment at multiple locations. The pre-op and post-op OCT images were taken along a plane transverse to the slits. A portion of the cornea in FIG. 34A appears upside down due to aliasing. A chilled sapphire lens was used to spare the corneal epithelium as described herein.

FIGS. 35A and 35B show OCT images of the pre-operative and post-operative locations respectively, of femto-slits in another ex vivo porcine eye with STEM-induced translocation (Δums) from a pre-op peak location to a post-op peak location via 360 degree annular treatment at multiple locations. The pre-op and post-op OCT images were taken along a plane transverse to the slits. A chilled sapphire lens was used to spare the corneal epithelium as described herein.

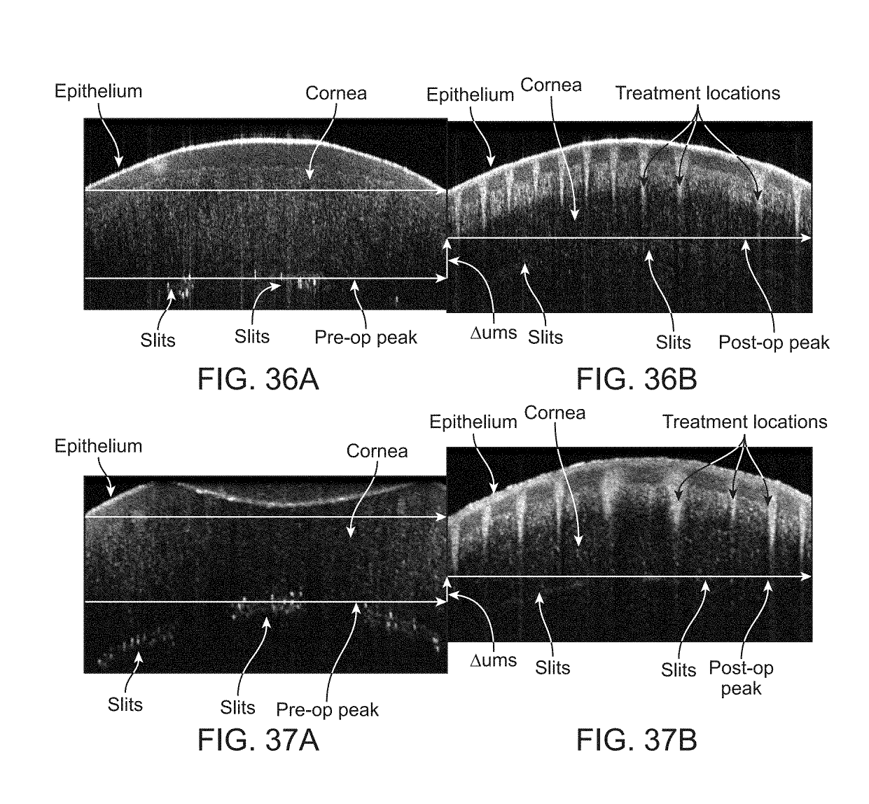

FIGS. 36A and 36B show OCT images of the pre-operative and post-operative locations respectively, of femto-slits in another ex vivo porcine eye with STEM-induced translocation (Δums) from a pre-op peak location to a post-op peak location via 360 degree annular treatment at multiple locations. The pre-op and post-op OCT images were taken along a plane transverse to the slits. A chilled sapphire lens was used to spare the corneal epithelium as described herein.

FIGS. 37A and 37B show OCT images of the pre-operative and post-operative locations respectively, of femto-slits in yet another ex vivo porcine eye with STEM-induced translocation (Δums) from a pre-op peak location to a post-op peak location via 360 degree annular treatment at multiple locations. The pre-op and post-op OCT images were taken along a plane transverse to the slits. A portion of the cornea in FIG. 37A appears upside down due to aliasing. A chilled sapphire lens was used to spare the corneal epithelium as described herein.

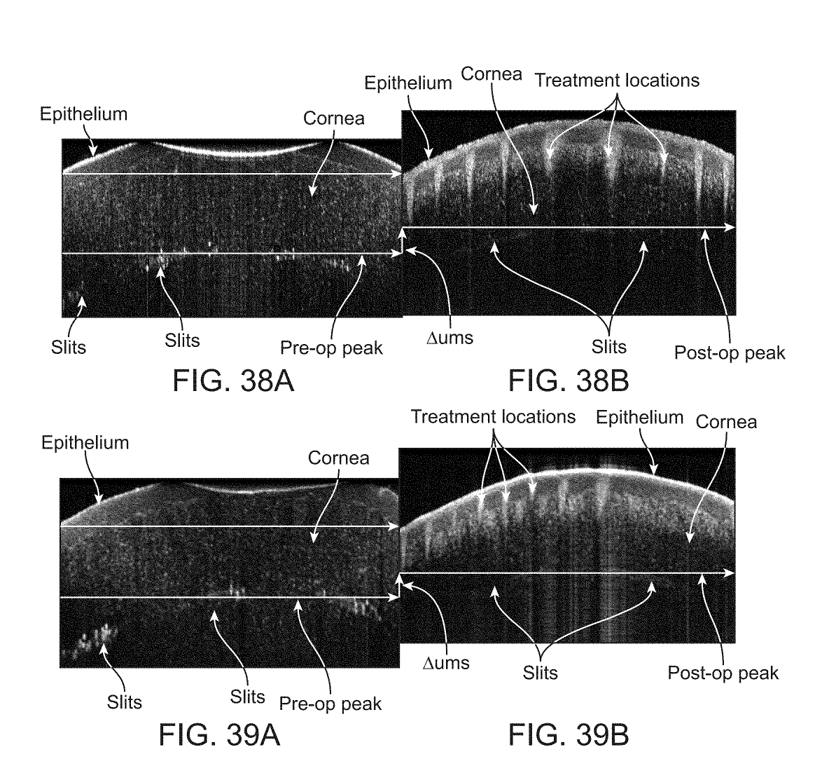

FIGS. 38A and 38B show OCT images of the pre-operative and post-operative locations respectively, of femto-slits in another ex vivo porcine eye with STEM-induced translocation (Δums) from a pre-op peak location to a post-op peak location via 360 degree annular treatment at multiple locations. The pre-op and post-op OCT images were taken along a plane transverse to the slits. A portion of the cornea in FIG. 38A appears upside down due to aliasing. A chilled sapphire lens was used to spare the corneal epithelium as described herein.

FIGS. 39A and 39B show OCT images of the pre-operative and post-operative locations respectively, of femto-slits in still another ex vivo porcine eye with STEM-induced translocation (Δums) from a pre-op peak location to a post-op peak location via 360 degree annular treatment at multiple locations. The pre-op and post-op OCT images were taken along a plane transverse to the slits. A portion of the cornea in FIG. 39A appears upside down due to aliasing. A chilled sapphire lens was used to spare the corneal epithelium as described herein.

FIGS. 40A and 40B show a zoomed-in view of OCT images of the pre-operative and post-operative locations respectively, of femto-slits in an ex vivo porcine eye with STEM-induced translocation via 360 degree annular treatment at multiple locations. The pre-op and post-op OCT images were taken along a plane parallel to a longitudinal axis of one of the canal-mimicking slits in order to highlight the undulating displacement of the slits between treatment annuli. A portion of the cornea in FIGS. 40A and 40B appear upside down due to aliasing. A chilled sapphire lens was used to spare the corneal epithelium as described herein. Treatment energy was delivered to the cornea to induce 360 annular shrinkage at treatment locations spaced about 1.4 mm radially apart. The shrinkage of the cornea above the canal-like slits induced axial strain and caused the slits to anteriorly translocate. Anterior translocation was most pronounced between treatment annuli, with a peak to peak distance of about 60 um measured in the slits after treatment, suggesting that Schlemm's canal may be widened by treating radially inward and/or outward of the roof of Schlemm's canal (i.e. “straddling” Schlemm's canal or juxtacanalicular). For example, two annuli about 1.5 mm apart may be positioned to straddle Schlemm's canal in order to increase the diameter (and volume) of Schlemm's canal as described herein.

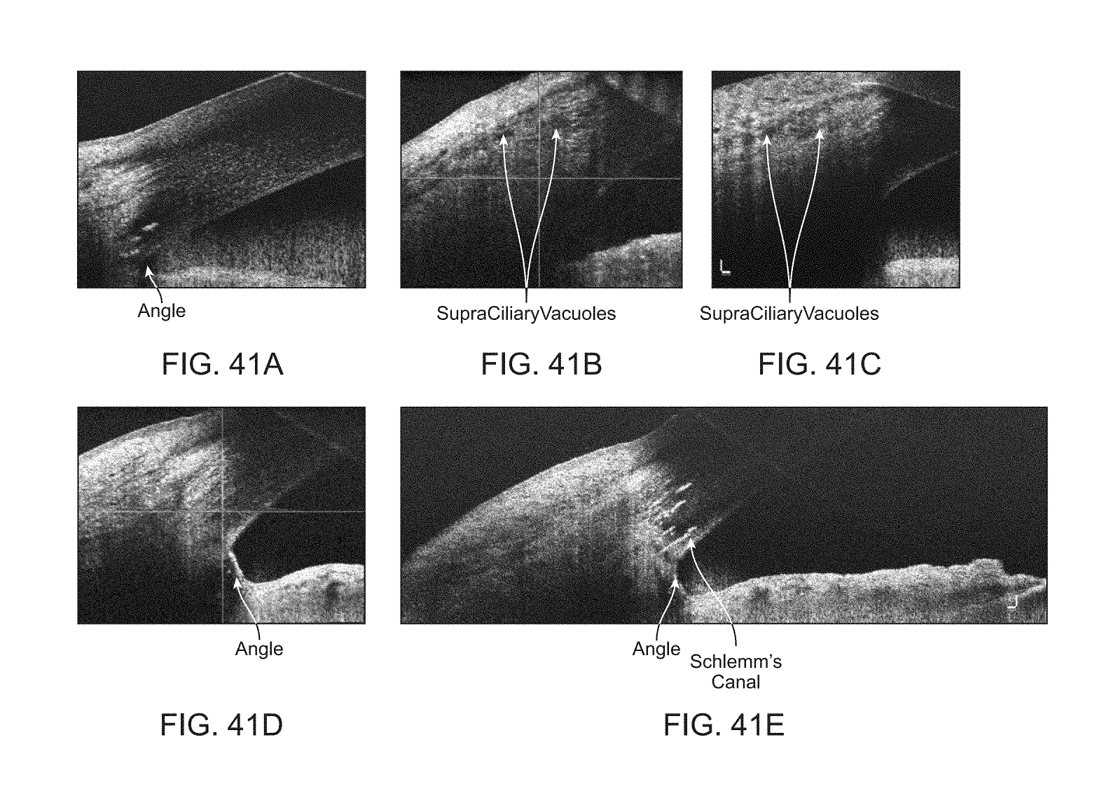

FIGS. 41A-41E show OCT images of a porcine eye treated ex vivo with two 360 degree juxtacanalicular shrinkage annuli approximately straddling Schlemm's canal and the trabecular meshwork. A continuous wave infra-red laser (approximately 2 um) was used to deliver energy to the eye through a chilled sapphire contact lens. Treatment energy was delivered sub-conjunctively in two 360 degree para-limbal annuli in order to open Schlemm's canal. The inner annulus was approximately 1 mm radially inward from the limbus and the outer annulus was approximately 1 mm radially outward from the limbus. Treatment was patterned to augment three pathway blockages as well as miosis by opening Schlemm's canal, opening angle/plateau, and opening supraciliary vacuoles. Additional treatment parameters and system information are described in Table 4. FIG. 41A shows an OCT image of a porcine eye prior to sub-conjunctiva 360 degree STEM shrinkage treatment about the limbus above the roof of Schlemm's canal in order to open Schlemm's canal and the trabecular meshwork and within the sclera to increase porosity and dilate vacuoles of the perilimbic sclera. FIGS. 41B-41E show OCT images of the eye of FIG. 41A after STEM treatment to open the angle, dilate supraciliary vacuoles of the sclera, and open Schlemm's canal. FIGS. 41B and 41C show dilation of vacuoles of the perilimbic sclera (black spots). FIG. 41D shows an increase in the anterior chamber angle compared to the pre-treatment angle of FIG. 41A. FIG. 41E shows an increase in the anterior chamber angle as well as opening of Schlemm's canal (black oval) following treatment.

TABLE 4
Glaucoma treatment parameters which may be used to open
Schlemm's canal and reduce intraocular pressure.

| Parameter | STEM |
|---|---|
| Energy (total) | 4 joules to 90 joules |
| Treatment time | 8 secs to 100 secs |
| Focal (fixation and scan) accuracy | Very high |
| Pre-op planning, intra-op stability | Very high |
| Epithelium/Conjunctiva/Vein/ | High |
| Ciliary/Pain Sparing | |
| Pre-programmed patterns with | Yes |
| patient-specific Schlemm's Canal | |
| diameters and vasculature | |
| based “stay out” zones | |
| Re-treatable, repeatable surgeries | Good (about 1.5 um/2 um |
| (related to absorption wavelength | continuous wave infrared laser |
| and focus) | scanner) |
| Reimbursable | (to be determined - likely) |
| Indications | IOP reduction (20%), pre-cataract |
| Cost | (to be determined but less than |
| | $20,000) |

Three patients were treated using 360 degree annular thermal shrinkage treatment to open Schlemm's canal, open angle, and increase scleral porosity/open supraciliary vacuoles. One eye of each patient was treated. Six annuli were treated on the sclera to about 20 mm from the center of the eye. The first and second annuli were juxtacanalicularly positioned to straddle the limbus (and Schlemm's canal) in order to dilate Schlemm's canal. The first annulus was positioned 1 mm radially inward of the minimum angle to angle diameter of the limbus. Two repetitions of 360 degree treatment with a 2.1 um, 200 mW laser at a scan speed of 5 mm/sec were performed at the first annulus location. The second annulus was positioned 1 mm radially outward of the maximum angle to angle diameter of the limbus. Two repetitions of 360 degree treatment with a 2.1 um, 200 mW laser at a scan speed of 5 mm/sec were performed at the second annulus location. The first and second annuli were positioned juxtacanalicularly so as to straddle the limbus to cause Schlemm's canal to open as described herein. The third through sixth annuli were positioned so as to create vacuoles in the sclera. The third through sixth annuli were patterned to provide annuli starting at 2 mm radially outward from the second annulus and ending at about 18 mm from the center of the eye, with a maximum of 1 mm between each annulus. Two repetitions of 360 degree treatment with a 2.1 um, 200 mW laser at a scan speed of 10 mm/sec were performed for each of the third through sixth annuli. The third through sixth annuli were patterned so as to generate or expand scleral vacuoles as described herein. The spot size used was 600 um for each annulus. Energy was delivered to the eye of each patient through a chilled sapphire contact lens in order to spare the epithelium as described herein. For the vacuole treatment patterns, the inventors believe that the combination two repetitions of the 10 mm/sec scan rate, 200 mW laser power, and 600 um diameter beam generated vacuoles in the sclera and likely without substantially effecting deeper tissues beneath the sclera, for example without substantially affecting tensioning of zonules of the lens of the eye.

The intraocular pressure of each patient was measured before and after glaucoma treatment. After treatment, eye drops containing trehalose and hyaluronate were provided to each patient for collagen stabilization and sequestration to improve recovery after therapy. The IOP of one eye of the first patient went from 21 mmHg before treatment to 12 mmHg 4 days after treatment. The IOP of one eye of the second patient went from 21 mmHg pre-treatment to 13 mmHg after pressure-reducing medication and shrinkage treatment. The IOP remained at 13 mmHg after stopping the medication post-treatment, indicating that STEM treatment provided for a stable 8 mmHg reduction in IOP. The IOP of one eye of the third patient went from 23 mmHg pre-treatment to 15 mmHg after pressure-reducing medication and decentered shrinkage treatment. The patient's eye was docked with the patient interface centered on the center of the eye as described herein. Treatment was decentered after docking, without changing the orientation of the eye within the patient interface, so as to be generally centered on the limbus. The IOP increased to 16 mmHg after stopping the medication post-treatment, indicating that STEM treatment provided for a stable 8 mmHg reduction in IOP. In the second and third patients, STEM treatment sufficiently improved IOP so as to allow for the stoppage of pressure-reducing medication. None of the patients had visible burns on the sclera or reported intraoperative or postoperative pain.

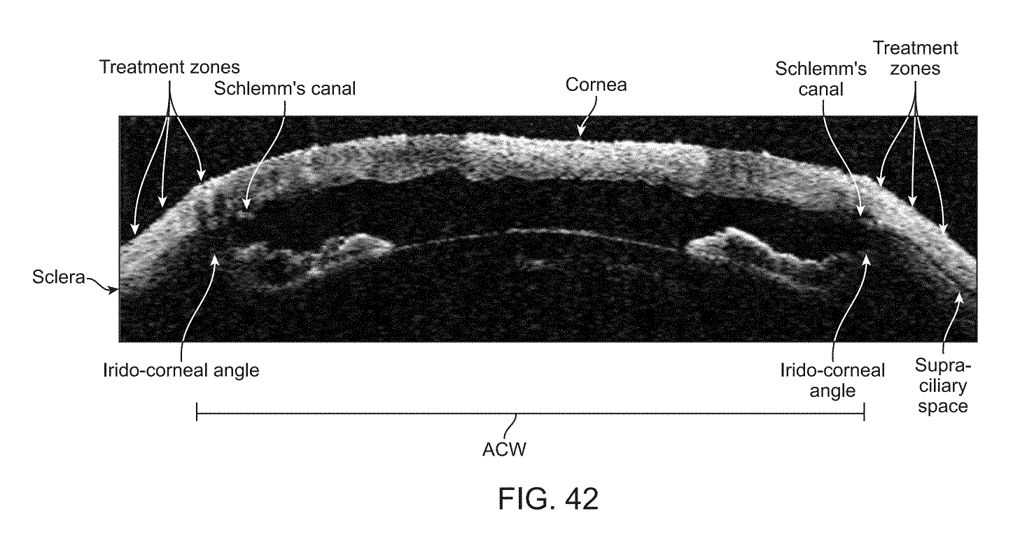

FIG. 42 shows an OCT image of a porcine eye treated ex vivo using the STEM glaucoma treatment system described herein. Treatment was patterned for 360 degrees in an annulus between 10 mm and 14 mm from the center of the eye in order to open the irido-corneal angle (also referred to herein as anterior chamber angle). Multiple repetitions were performed to increase the amount of energy deposited to the treatment location. The laser power was 300 mW. The laser power and total energy deposited was higher than might be used in patients (which may be closer to 200 mW) but no damage was observed when the energy was delivered through a chilled contact lens contacting the eye and the depth of treatment did not exceed mid-stroma of the cornea. After treatment, Schlemm's canal appeared to be dilated compared to pre-treatment (not shown) and the irido-corneal angle was opened. The bend of the iris also appeared to change, which may indicate the relevance of the treatment systems and methods described herein for opening angle in iris plateau syndrome which can lead to glaucoma. The meridional anterior chamber width (“ACW”) or angle to angle distance changed from pre- to post-treatment indicating that ACW may be useful for pre-operative canal mapping. ACW, which differs between patients, may be used to derive treatment patterns in a manner similar to the use of the limbus described herein. The limbus may in fact be used to estimate the ACW manually or using OCT for treatment patterning. Vacuoles (black spots in the sclera) were also dilated, as was the superciliary space, following treatment. Axial pachymetry was also assessed after treatment of the center of the cornea in a 360 degree annulus spanning 1 mm to 3 mm from the center of the eye with multiple repetitions with a laser power of 300 mW. Following aggressive treatment, axial pachymetry indicated that corneal shrinkage/thinning was about 5% after treatment.

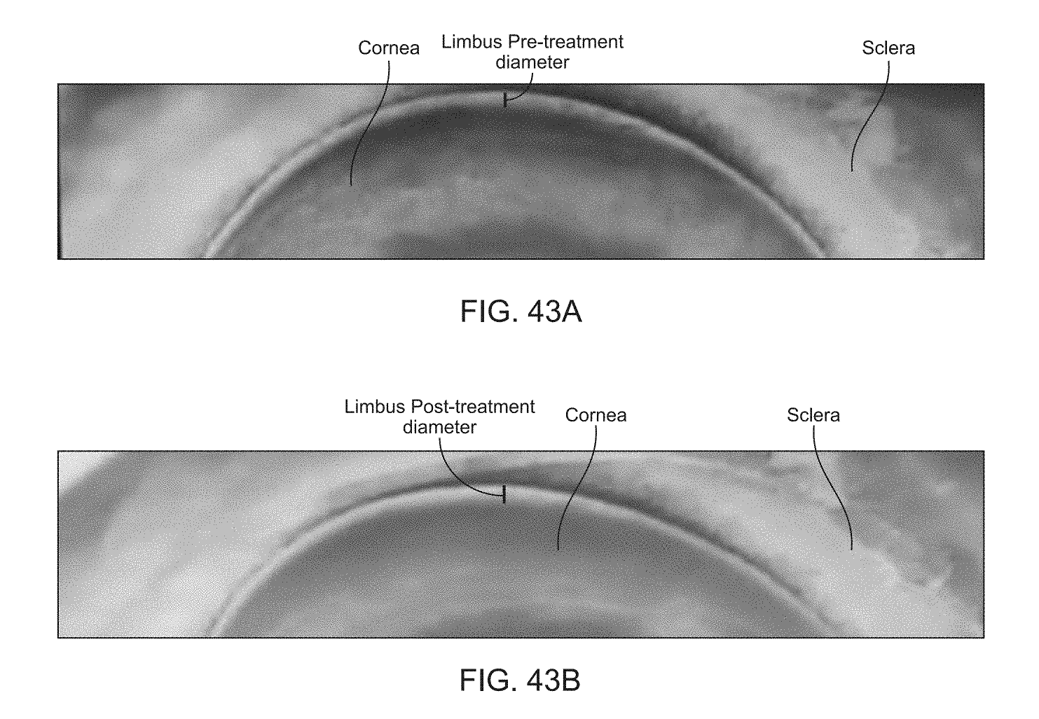

FIGS. 43A-43B show the diameter of the limbus of an ex vivo porcine eye, as a reference for Schlemm's canal, before and after paralimbal treatment of Schlemm's canal. Four eyes were treated ex vivo with the laser (STEM) system described herein using laser powers ranging from 250 mW to 400 mW and scan speeds ranging from 5 mm/sec to 10 mm/sec. Two to eight repetitions of the annular scan pattern were performed. Treatment was patterned at 11 mm or 15 mm from the center of the eye depending on the location of the limbus of the eye being treated. A 360 degree circular scan pattern was used for treatment, though in many cases the limbus (and thus Schlemm's canal) was asymmetrical (neither circular, oval, nor elliptical). Use of a scan pattern more closely resembling the perimeter of Schlemm's canal and/or the limbus may provide for further dilation of Schlemm's canal as described herein. Laser energy was delivered to each eye through a chilled contact lens in order to spare the surface of the cornea from damage and prevent surface opacification. FIG. 43A shows an anterior image of a portion of an eye before treatment and FIG. 43B shows an anterior image of a portion of the eye after treatment. A change in diameter of the limbus may be used as a surrogate for a change in diameter (i.e. opening) of Schlemm's canal. The diameter was measured by eye as the “white-to-white” distance of the libel “stripe”. Annular treatment of the eye resulted in an increase in the diameter of the limbus in each of the eyes treated. OCT images (not shown) were taken of the eyes to confirm opening of Schlemm's canal as a result of treatment. Angle opening was also observed following treatment, suggesting that annular treatment may be used to treat glaucoma by improving fluid flow in multiple ways.

Table 5 shows the results of a similar proof of concept experiment. A 2.01 um laser was used to scan a paralimbal 360 degree circular annulus on each of five porcine eyes ex vivo. Brightfield anterior images of the eyes pre- and post-treatment were captured and the diameter of the limbus (measured as the “white-to-white” distance of the limbal “stripe”) was measured for each image. A change in diameter in the limbus (and sclera) may infer a change in diameter of Schlemm's canal. The change in diameter may suggest a squared response in the volume after treatment. Treatment annuli were visually positioned near the limbus of the eyes and energy was delivered to the tissue through a chilled scleral contact less. The change in diameter of the limbus, and by association Schlemm's canal, showed a widening/opening increase of about 20% after treatment.

TABLE 5
Measured change in diameter of the limbus using
“white-to-white” measurement.

| Exp. | Change in Limbal Diam. |
|---|---|
| 1 | 7% |
| 2 | 20% |
| 3 | 34% |
| 4 | 29% |
| 5 | 12% |

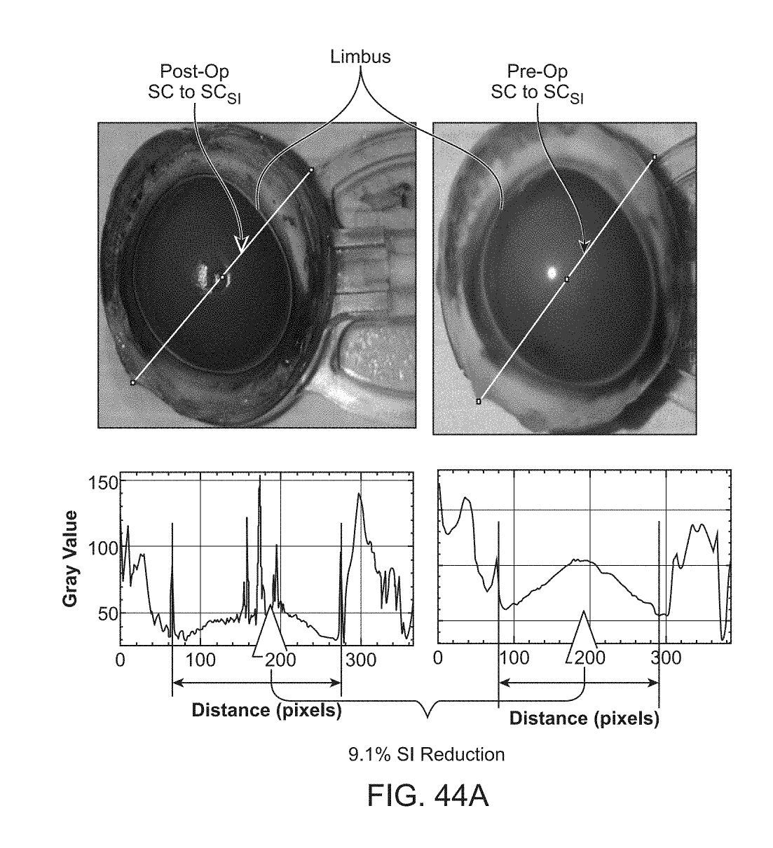

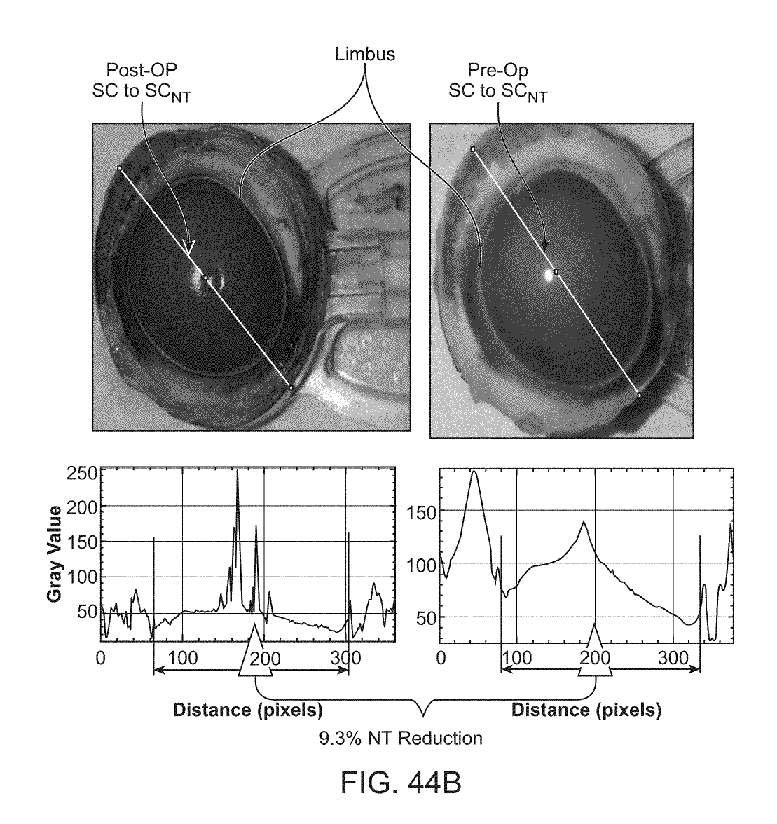

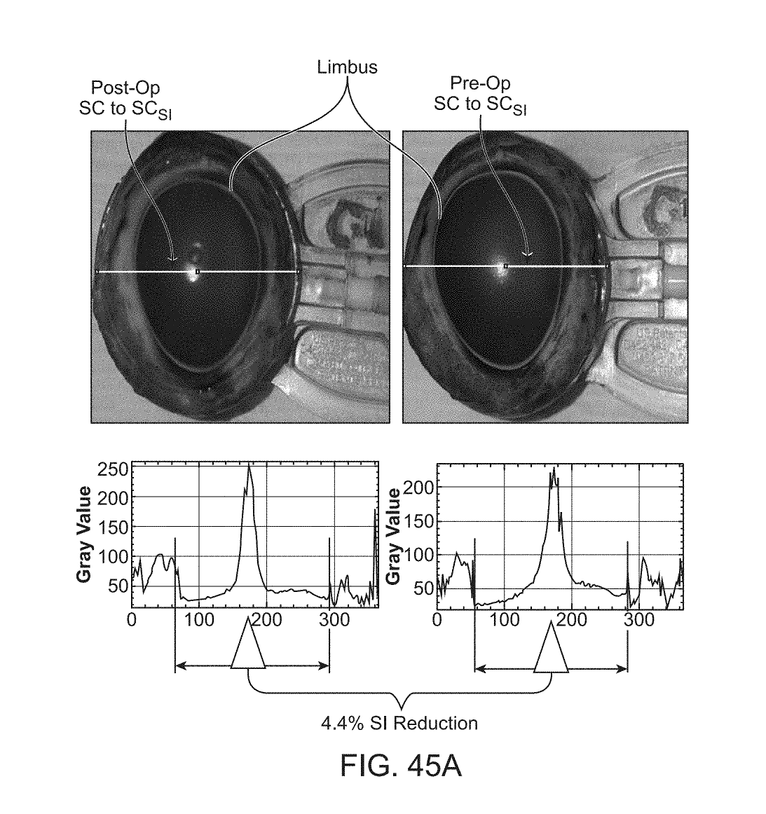

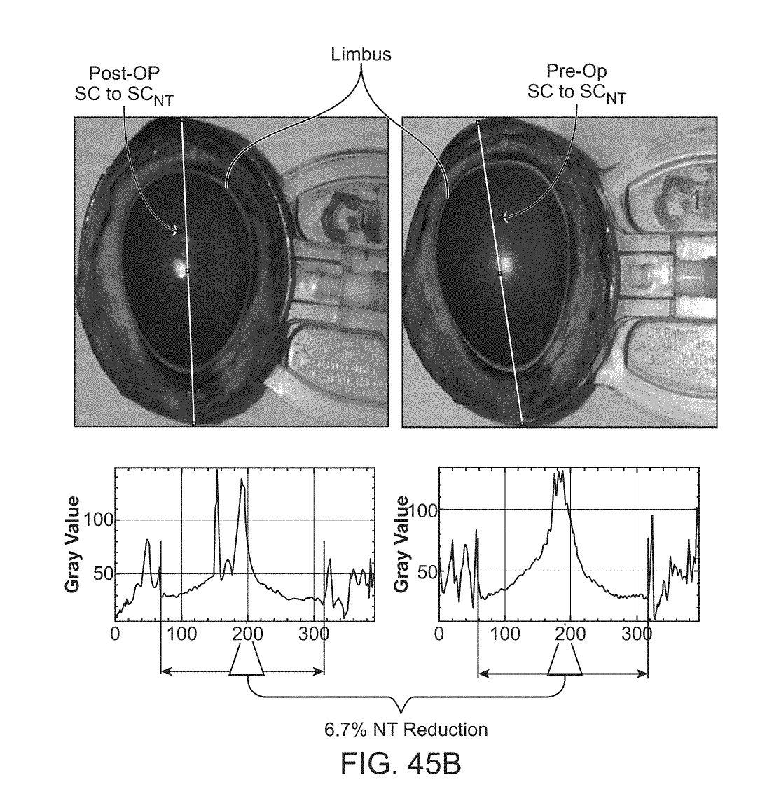

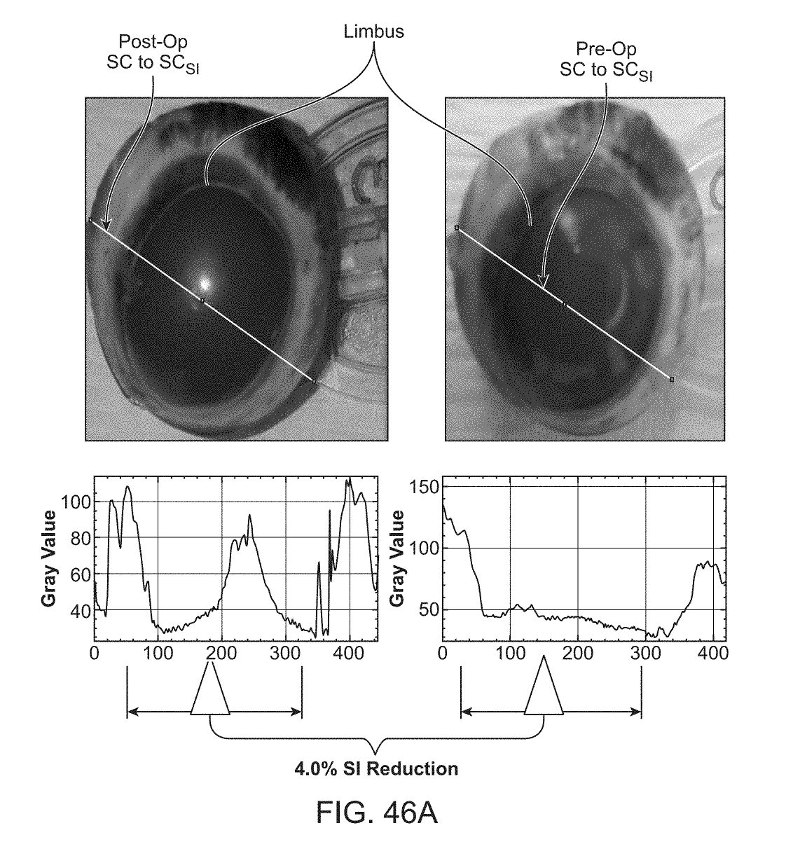

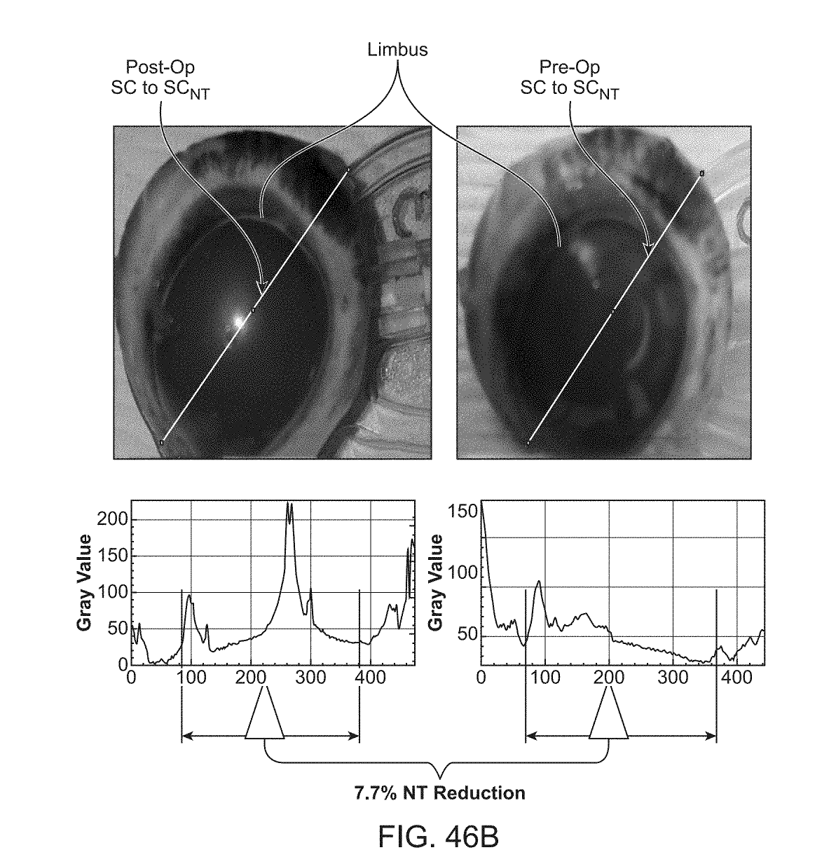

FIGS. 44A-46B show the results of 360 annular treatment radially inward of the limbus in order to widen Schlemm's canal. Three porcine eyes were each treated ex vivo for 2 minutes with a laser power of 250 mW and a scan speed of 10 mm/sec with a 2 mm thick 360 degree annulus positioned 0.2 mm radially inward from the edge of the limbus/Schlemm's canal. The 2 mm thick annulus comprised a plurality of annuli spaced 0.2 mm apart. Each annulus was shaped to mimic (e.g. correspond to) the shape of the limbus. The first and second eyes (see FIGS. 44A-45B) were treated with a 2.01 um laser. The third eye (see FIGS. 46A-46B) were treated with a 1.47 um laser. ImageJ (NIH) was used to determine the change in diameter of the limbus (and by extension Schlemm's canal) in images of post-op eyes compared to images of pre-op eyes. A line was drawn across the eye (including two locations of the limbus along its length) of the pre- and post-treatment images for each eye and the pixel intensity along each line was determined using the “plot profile of line selection” feature. The pixel intensities along the line was used to define the inner boundary of the limbus at each of the points where the line crossed the limbus and the distance (or chord length) between the two inner boundaries, “SC to SC”, was calculated. SI stands for lines oriented in the superior-inferior direction and NT stands for lines oriented in the nasal-temporal direction. A typical pre-op SC to SC may be about 12 mm and the pre-op width of Schlemm's canal may be about 0.5 mm. A change in chord length pre-op to post-op was taken to represent twice the change in the diameter (and/or position) of the limbus/Schlemm's canal and indicated a widening and/or translocation of Schlemm's canal. Widening and/or translocation of Schlemm's canal may widen the trabecular meshwork and/or open the anterior chamber angle, thereby improving outflow and treating glaucoma. FIGS. 44A and 44B show results of a first eye treated to widen Schlemm's canal. SC to SC_{SI} was reduced by 9.1% and SC to SC_{NT} was reduced by 9.3% pre-op to post-op, indicating an inward translocation and/or widening of the limbus and Schlemm's canal with treatment. Given a typical width of 0.5 mm, Schlemm's canal was calculated to have widened by an extra inner edge stretch of 0.55 mm and 0.56 mm in the SI and NT directions, respectively. FIGS. 45A and 45B show results of a second eye treated to widen Schlemm's canal. SC to SC_{SI} was reduced by 4.4% and SC to SC_{NT} was reduced by 6.7% pre-op to post-op. Schlemm's canal was calculated to have widened by an extra inner edge stretch of 0.26 mm and 0.40 mm in the SI and NT directions, respectively. FIGS. 46A and 46B show results of a third eye treated to widen Schlemm's canal. SC to SC_{SI} was reduced by 4.0% and SC to SC_{NT} was reduced by 7.7% pre-op to post-op. Schlemm's canal was calculated to have widened by an extra inner edge stretch of 0.26 mm and 0.46 mm in the SI and NT directions, respectively. Taken together, these results suggest a doubling, on average, of the width of Schlemm's canal after treatment.

Table 6 shows the results of a similar proof of concept experiment. Eleven porcine eyes were treated ex vivo with a 360 degree paralimbal circular pattern near the limbus using a 2.01 um laser. Treatment patterns were manually positioned on each eye. Treatment energy was delivered to the eye through a chilled scleral contact lens. Images were taken pre- and post-op (not shown) and a line was drawn across each image to measure changes in the diameter of the limbus using pixel intensities along the line to determine changes in chord length as described herein. The change in limbal diameter or inner edge stretch indicated a translocation or widening of the limbus (and Schlemm's canal) of about 175% on average.

TABLE 6
Measured change in diameter of the limbus using intensities
to determine change in chord length.

| Exp. | Change in Limbal Diam. |
|---|---|
| 1 | 209% |
| 2 | 212% |
| 3 | 152% |
| 4 | 181% |
| 5 | 192% |
| 6 | 118% |
| 7 | 146% |
| 8 | 146% |
| 9 | 229% |
| 10 | 192% |
| 11 | 146% |

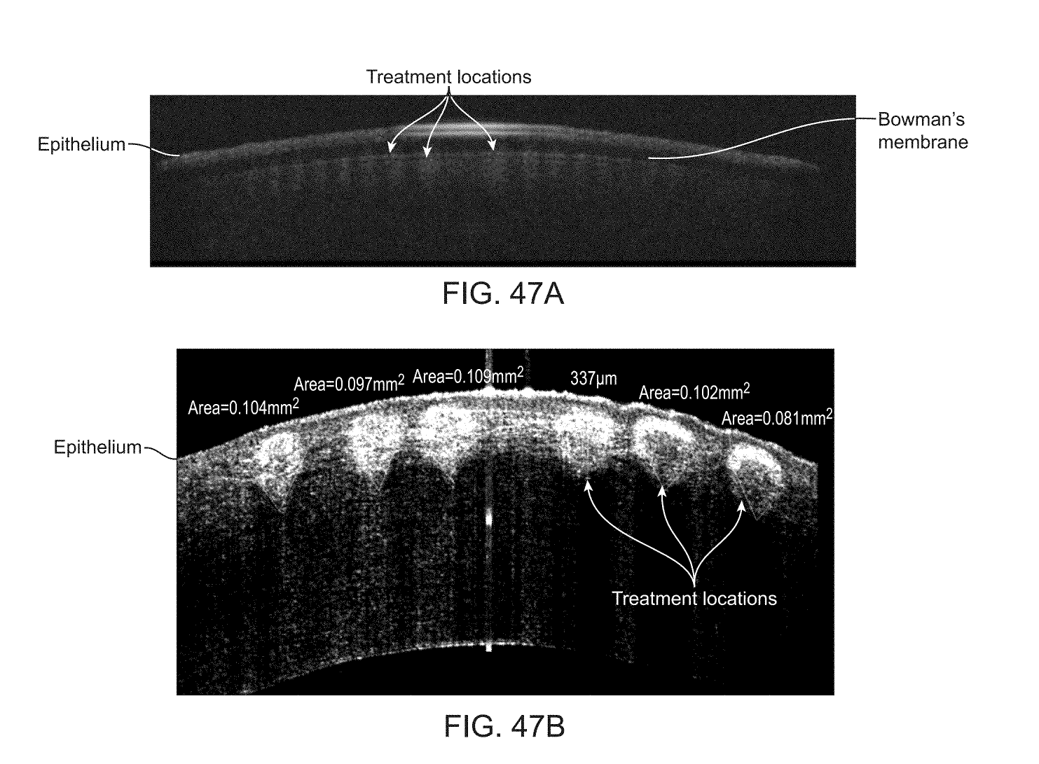

FIGS. 47A-47B show OCT images of porcine eyes treated at multiple locations ex vivo with the laser system described herein. Laser energy was delivered through a chilled sapphire contact lens as described herein in order to spare the surface tissue of the cornea. FIG. 47A shows that treatment directed energy into the cornea while sparing the corneal epithelium and Bowman's membrane. FIG. 47B shows that the corneal epithelium was able to be spared using the STEM glaucoma treatment system and methods described herein even with aggressive treatment.

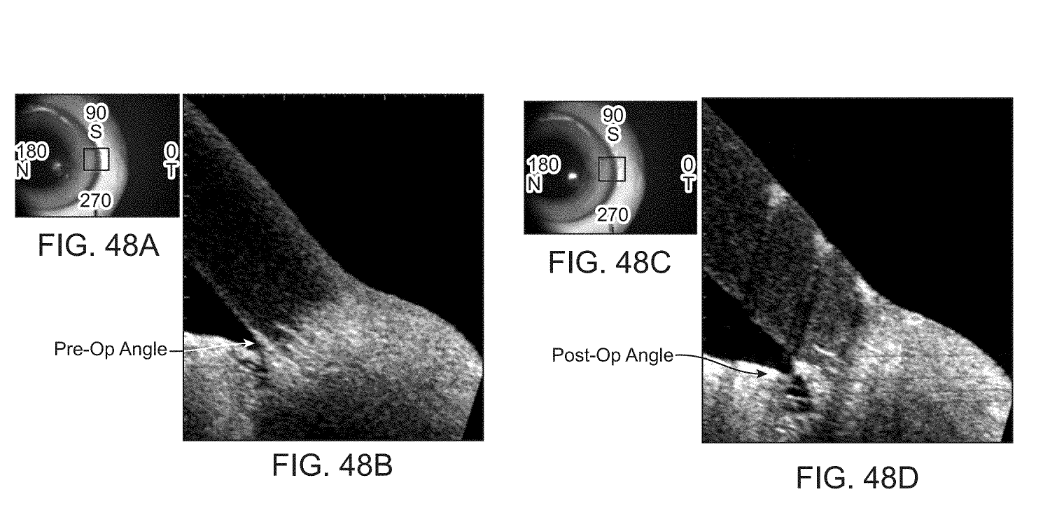

FIGS. 48A-48B show pre-operative images of a porcine eye. FIGS. 48C-48D show post-operative images of the eye of FIGS. 48A-48B treated with the STEM system and methods described herein. The eye was treated ex vivo using a 2.01 um laser with a laser power of 400 mW and a scan speed of 20 mm/sec with a 3 mm thick 360 degree annulus positioned between 12 mm and 15 mm from the center of the eye. The 3 mm thick annulus comprised a plurality of annuli spaced 1 mm apart. 32 repetitions were performed at each annular ring. Each annulus was shaped to correspond to (e.g. mimic) the shape of the limbus. FIGS. 48A and 48C show brightfield images of the eye face-on with boxes indicating the location which was imaged using OCT to generate FIGS. 48B and 48D, respectively. After treatment, the irido-corneal angle was opened.

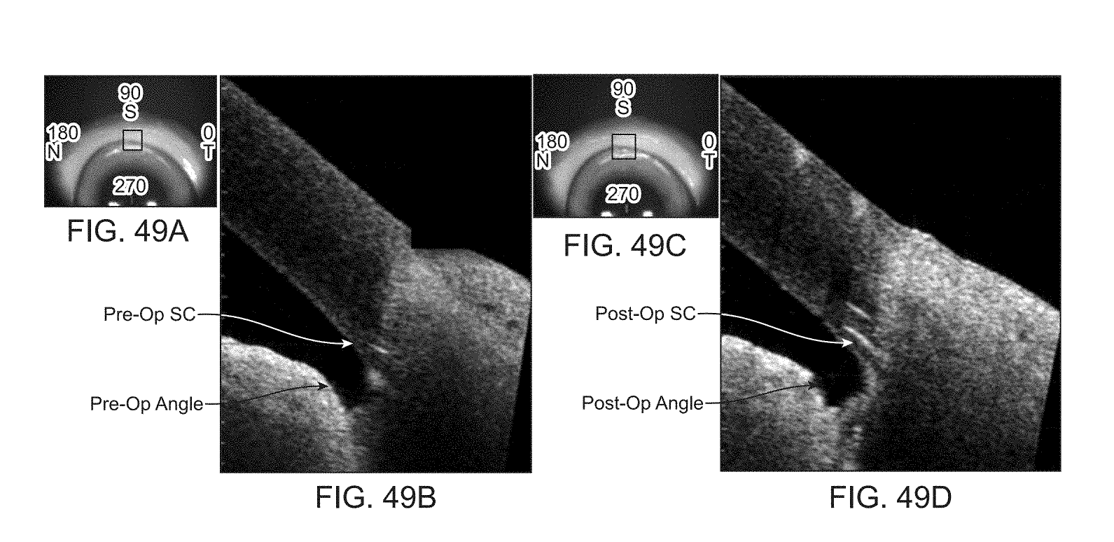

FIGS. 49A-49B show pre-operative images of a porcine eye. FIGS. 49C-49D show post-operative images of the eye of FIGS. 49A-49B treated with the STEM system and methods described herein. The eye was treated ex vivo using a 2.01 um laser with a laser power of 400 mW and a scan speed of 20 mm/sec with a 3 mm thick 360 degree annulus positioned between 12 mm and 15 mm from the center of the eye. The 3 mm thick annulus comprised a plurality of annuli spaced 1 mm apart. 32 repetitions were performed at each annular ring. Each annulus was shaped to mimic the shape of the limbus. FIGS. 49A and 49C show brightfield images of the eye face-on with boxes indicating the location which was imaged using OCT to generate FIGS. 49B and 49D, respectively. After treatment, Schlemm's canal appeared to be dilated compared to pre-treatment and the irido-corneal angle was opened.

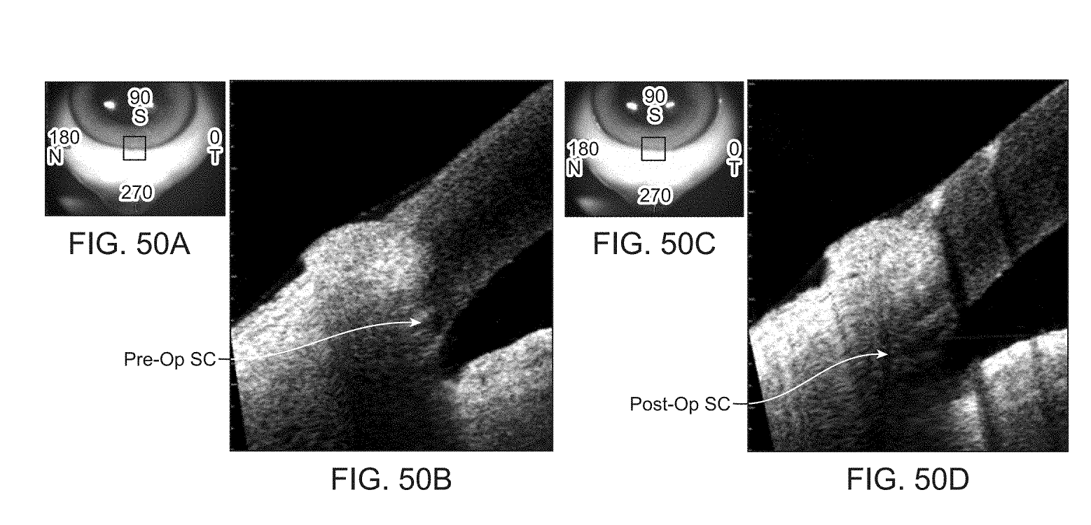

FIGS. 50A-50B show pre-operative images of a porcine eye. FIGS. 50C-50D show post-operative images of the eye of FIGS. 50A-50B treated with the STEM system and methods described herein. The eye was treated ex vivo using a 2.01 um laser with a laser power of 400 mW and a scan speed of 20 mm/sec with a 3 mm thick 360 degree annulus positioned between 12 mm and 15 mm from the center of the eye. The 3 mm thick annulus comprised a plurality of annuli spaced 1 mm apart. 32 repetitions were performed. Each annulus was shaped to mimic the shape of the limbus. FIGS. 50A and 50C show brightfield images of the eye face-on with boxes indicating the location which was imaged using OCT to generate FIGS. 50B and 50D, respectively. After treatment, Schlemm's canal appeared to be dilated compared to pre-treatment.

Although the above experiments were conducted on ex vivo porcine eyes, a person of ordinary skill in the art can conduct in vivo experiments on living human eyes in order to develop nomograms to position the lens of the eye to correct vision with treatment energies as described herein. For example, the laser system can be coupled to an OCT measurement systems as described herein, and OCT measurements on living human eyes during surgery can be used to measure movement of the lens in response to treatment as described herein, in order to determine amounts and locations of energy treatment that produce desired amounts of movement of the lens. The OCT system can be coupled to the laser system with a beam splitter, such that the laser beam path and OCT measurement beam path are aligned, and the patient can be measured in situ. Also, although treatment dimensions are provided with reference to porcine eyes, which typically have dimensions 10 to 20% greater (or more) than human eyes, a person of ordinary skill in the art can target similar structures with human eyes by decreasing the dimensions of porcine eyes by approximately 10 to 20% to target similar tissue structures on human eyes. Additionally, porcine eyes typically comprise different types and amounts (or ratios of collagen types) than human eyes and a person of ordinary skill in the art can adjust the treatment parameters to account for such differences.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. A system for treating glaucoma of an eye, the system comprising:
a processor configured with instructions to:
receive input corresponding to a plurality of locations of a Schlemm's canal of the eye, and
generate a plurality of treatment locations for the eye in response to the plurality of locations, wherein the plurality of treatment locations is located within 2 mm of the Schlemm's canal, and located radially inward from the Schlemm's canal toward an optical axis of the eye and radially outward from the Schlemm's away from the optical axis of the eye;
an energy source configured to generate energy to treat the eye; and
a scanner operably coupled to the energy source and the processor, the scanner configured to deliver the energy to the plurality of treatment locations in order to stretch the Schlemm's canal of the eye.

2. The system of claim 1, wherein the plurality of treatment locations comprise juxtacanalicular locations located within 2 mm of the Schlemm's canal and wherein the plurality of treatment locations extend in a first annular treatment pattern on a first side of the Schlemm's canal and a second annular treatment pattern on a second side of Schlemm's canal opposite the first side in order stretch tissue between the first annular treatment pattern and the second annular treatment pattern to dilate the Schlemm's canal and increase porosity of a trabecular meshwork of the eye.

3. The system of claim 2, wherein the first annular treatment pattern is located radially inward from Schlemm's canal and the second annular treatment pattern is located radially outward from Schlemm's canal relative to the optical axis of the eye.

4. The system of claim 2, wherein the processor is configured with instructions to shrink tissue with the first annular treatment pattern prior to shrinking tissue with the second annular treatment pattern.

5. The system of claim 2, wherein the processor is configured with instructions to shrink tissue with at least a portion of the first annular treatment pattern prior to shrinking tissue with at least a portion of the second annular treatment pattern.

6. The system of claim 2, wherein the first annular treatment pattern comprises a first plurality of spaced apart annular treatment patterns and the second annular treatment pattern comprises a second plurality of spaced apart annular treatment patterns.

7. The system of claim 6, wherein the first plurality of spaced apart treatment annular treatment patterns comprises angularly separated spaced apart treatment patterns and the second plurality of annular treatment pattern comprises angularly separated spaced apart treatment patterns.

8. The system of claim 6, wherein the first plurality of spaced apart treatment annular treatment patterns comprises radially separated spaced apart treatment patterns and the second plurality of annular treatment pattern comprises radially separated spaced apart treatment patterns.

9. The system of claim 2, wherein the processor is configured with instructions to configured repeatedly deliver the energy to each the plurality of treatment locations with a time delay in order to fractionate delivery of energy to said each of the plurality of treatment locations.

10. The system of claim 9, wherein the time delay is within a range from about 10 millisecond (ms) to about 60 seconds and optionally wherein the time delay is within a range from about 100 ms to about 30 seconds and optionally within a range from about 500 ms to about 15 seconds and optionally within a range from about 1 second (s) to about 10 seconds.

11. The system of claim 2, wherein the processor coupled to the energy source and the scanner is configured with instructions to heat tissue at the plurality of treatment locations to a temperature within a range from 60 to 70 degrees Centigrade at a depth within a range from 50 to 400 microns at each of the plurality of treatment locations along the first annular pattern and the second annular pattern.

12. The system of claim 11, wherein a majority of a treatment energy of the first treatment pattern is located within 1.5 mm of the Schlemm's canal and a majority of treatment energy of the second pattern is located within 1.5 mm of Schlemm's canal.

13. The system of claim 12, wherein the first annular treatment pattern and the second annular treatment pattern are configured to open an angle of the eye by an amount within a range from 1 to 6 degrees.

14. The system of claim 13, wherein the first annular treatment pattern extends at least about 30 degrees around the optical axis of the eye and the second annular treatment pattern extends at least about 30 degrees around the optical axis of the eye.

15. The system of claim 14, wherein the first treatment annular treatment pattern extends at least about 40 degrees around the optical axis of the eye and the second treatment pattern extends at least about 40 degrees around the optical axis of the eye.

16. The system of claim 15, wherein the first annular treatment pattern and the second annular treatment pattern may be arranged avoid heating tissue overlaying the Schlemm's canal.

17. The system of claim 16, wherein the first annular treatment pattern and the second annular treatment pattern comprise circular, oval, elliptical, egg-like, non-circular, non-elliptical, or asymmetrical, shapes patterned so as to correspond to the shape of Schlemm's canal or the limbus.

18. The system of claim 17, wherein the energy source comprises one or more of a pulsed laser, a continuous laser, a pulsed ultrasound transducer, a HIFU array, or a phased HIFU array.

19. The system of claim 1, the input comprises an input from a user of the system or an input from an imaging apparatus.

20. The system of claim 1, wherein the processor is configured with instructions to generate vacuoles in a sclera of the eye.

21. The system of claim 20, wherein the energy source comprises a laser having a wavelength within a range from about 1.9 to 2.3 microns.

22. The system of claim 20, wherein the energy source comprises a laser having a wavelength of about 1.9 microns.

23. The system of claim 20, wherein the processor is configured with instructions to scan laser beam with an annular pattern on the sclera.

24. The system of claim 23, wherein the processor is configured with instructions to scan the sclera with two repetitions of the annular pattern.

25. The apparatus of claim 1, wherein the processor is configured with instructions to treat the eye in order to increase an angle of the eye, dilate and stretch one or more of the trabecular meshwork or Schlemm's canal, increasing porosity of the sclera and dilating the perilimibic sclera, dilate collector channels, or dilate ostia of collector channels of the eye.

26. A method for treating glaucoma of an eye, the method comprising:
determining a plurality of locations of a Schlemm's canal of the eye; and
delivering energy to a plurality of treatment locations of the eye in response to the plurality of locations, wherein the plurality of treatment locations is located within 2 mm of the Schlemm's canal, and located radially inward from the Schlemm's canal toward an optical axis of the eye and radially outward from the Schlemm's canal away from the optical axis of the eye;
wherein the energy is delivered to the plurality of treatment locations in order to stretch the Schlemm's canal of the eye.

27. The method of claim 26, wherein the plurality of treatment locations comprise juxtacanalicular locations located within 2 mm of the Schlemm's canal and wherein the plurality of treatment locations extend in a first annular treatment pattern on a first side of the Schlemm's canal and a second annular treatment pattern on a second side of Schlemm's canal opposite the first side, and wherein the first annular treatment pattern is located radially inward from Schlemm's canal and the second annular treatment pattern is located radially outward from Schlemm's canal relative to the optical axis of the eye.

28. The method of claim 27, wherein the tissue shrinks with the first annular treatment pattern and the second annular treatment pattern in order stretch tissue between the first annular treatment pattern and the second annular treatment pattern to dilate the Schlemm's canal and increase porosity of a trabecular meshwork of the eye.

29. The method of claim 28, wherein the tissue is heated to a temperature within a range from 60 to 70 degrees Centigrade at a depth within a range from 50 to 400 microns at each of the plurality of treatment locations along the first annular pattern and the second annular pattern.

30. The method of claim 29, wherein a majority of a treatment energy of the first treatment pattern is located within 1.5 mm of the Schlemm's canal and a majority of treatment energy of the second pattern is located within 1.5 mm of Schlemm's canal.

31. The method of claim 30, wherein the first annular treatment pattern and the second annular treatment pattern are configured to open an angle of the eye by an amount within a range from 1 to 3 degrees.

32. The method of claim 31, wherein the first annular treatment pattern extends at least about 90 degrees around the optical axis of the eye and the second annular treatment pattern extends at least about 90 degrees around the optical axis of the eye.

33. The method of claim 31, wherein the first treatment annular treatment pattern extends at least about 180 degrees around the optical axis of the eye and the second treatment pattern extends at least about 180 degrees around the optical axis of the eye.

34. The method of claim 32, wherein the first annular treatment pattern and the annular second treatment are arranged avoid shrinking tissue overlaying the Schlemm's canal.

35. The method of claim 34, wherein the first annular treatment pattern and the second annular treatment pattern comprise circular, oval, elliptical, egg-like, non-circular, non-elliptical, or asymmetrical, shapes patterned so as to correspond to the shape of Schlemm's canal or the limbus.

36. The method of claim 35, wherein the energy comprises energy from one or more of a pulsed laser, a continuous laser, a pulsed ultrasound transducer, a HIFU array, or a phased HIFU array.

37. The method of claim 26, wherein further comprising generating vacuoles in a sclera of the eye.

38. The method of claim 37, wherein the energy comprises energy from a laser having a wavelength within a range from about 1.9 to 2.3 microns.

39. The method of claim 37, wherein a laser beam is scanned with an annular pattern on the sclera.

40. The method of claim 39, wherein the processor is configured with instructions to scan the sclera with two repetitions of the annular pattern.
